(19) United States
(12) Patent Application Publication
(10) Pub. No.: US 2025/0235138 A1
(43) Pub. Date: Jul. 24, 2025

(54) HEALTH INFORMATION SERVICE PROVIDING DEVICE FOR MOBILITY USER, AND HEALTH INFORMATION SERVICE PROVIDING DEVICE FOR RIDER

(71) Applicant: Sumitomo Riko Company Limited, Aichi (JP)

(72) Inventors: Koichi Hasegawa, Aichi (JP); Masaru Murayama, Aichi (JP)

(73) Assignee: Sumitomo Riko Company Limited, Aichi (JP)

(21) Appl. No.: 19/174,882

(22) Filed: Apr. 9, 2025

Related U.S. Application Data
(63) Continuation of application No. PCT/JP2023/039594, filed on Nov. 2, 2023.

(30) Foreign Application Priority Data
Dec. 14, 2022 (JP) 2022-199463
Dec. 26, 2022 (JP) 2022-208935

(51) Int. Cl. A61B 5/18; A61B 5/00; A61B 5/024; A61M 21/00; B60Q 9/00

(52) U.S. Cl. CPC A61B 5/18; A61B 5/02438; A61B 5/4836; A61B 5/6893; A61B 5/7282; A61M 21/00; B60Q 9/00; A61M 2021/0083

(57) ABSTRACT

A health information service providing device for a mobility user includes a sensor acquiring a biological signal of a mobility user, an estimation part estimating a heart rate signal related to a heart rate of the mobility user, a calculation part calculating VLF data related to a very-low frequency component, a state estimation part estimating a change in a health state of the mobility user based on a difference between current VLF data of the mobility user this time and past VLF data in a specific period in the past prior to this time, and a user interface providing a specific service to the mobility user based on the change in the health state of the mobility user.

### CROSS-REFERENCE TO RELATED APPLICATION

The present application is a continuation of PCT/JP2023/039594, filed on Nov. 2, 2023, and is related to and claims priority from Japanese patent application no. 2022-199463, filed on Dec. 14, 2022 and Japanese patent application no. 2022-208935, filed on Dec. 26, 2022. The entire contents of the aforementioned applications are hereby incorporated by reference herein.

### BACKGROUND

### Technical Field

The disclosure relates to a health information service providing device for a mobility user, and a health information service providing device for a rider.

### Related Art

Conventionally, as an indicator of a health state of a person, it has been a practice to measure a person's heart rate or pulse rate and use a very-low frequency component (VLF) calculated from the measured value. The very-low frequency component (hereinafter referred to as VLF) is susceptible to short-term fluctuations due to the influence of physical conditions such as fatigue and stress. Thus, in medical institutions, to obtain accurate information related to VLF, measurements of the heart rate or the pulse rate have been performed on subjects in a resting state. In other words, by measuring the heart rate or the pulse rate of a subject in a resting state, the state of the subject is accurately learned to obtain VLF values in an environment with little influence from external disturbances. Patent Literature 1 (Japanese Patent Application Laid-Open No. 2022-54332) describes a technique in which measurements of the heart rate or the pulse rate are performed on subjects in a sleeping state as an example of the resting state, to obtain stable information related to VLF.

In addition, conventionally, regarding drowsiness of a rider in a mobility exemplified by a vehicle, a technique has been disclosed to detect the rider's drowsiness in a relatively short time based on a time change in the rider's heartbeat interval and a predetermined determination criterion for the time change in the heartbeat interval (refer to Patent Literature 2: Japanese Patent Application Laid-Open No. 2018-192128). In the above technique, a service is provided to the rider of the mobility to notify the rider of a message corresponding to drowsiness by an image or a voice.

However, it will become easier to learn about the health state of the subject if VLF-related information can be easily measured as a part of the subject's activities in daily life, rather than measured under limited conditions with the subject being in a resting state or measured in special facilities for minimizing the influence from external disturbances.

Thus, it is conceivable to acquire changes in body movement of a mobility user riding in a mobility by a sensor, estimate the heart rate or the pulse rate from such changes in body movement, and calculate, from the estimated heart rate or pulse rate, a value (hereinafter referred to as VLF data) correlated with the VLF measured at rest.

However, driving conditions of the mobility change from moment to moment. For example, the psychological state of the mobility user changes when driving in a residential area with many narrow and winding roads, when driving in a busy city center, when driving on a highway, etc. Accordingly, information related to the heart rate or the pulse rate of the mobility user also changes according to changes in the driving conditions of the mobility. Thus, there is a problem that it is difficult to estimate the health state of the mobility rider based solely on data of a limited period during mobility riding.

In addition, in the case where drowsiness of the rider increases, it may be due to influence in living conditions of the rider in the past. For example, it is conceivable that sleep deprivation may have continued or physical fatigue may have accumulated over the past few days. In such cases, even in a state where drowsiness is not detected, the rider's drowsiness is still very likely to increase. Thus, it is desirable to improve accuracy of determining whether the rider's drowsiness exhibits an increase tendency based on the rider's past data related to drowsiness.

### SUMMARY

An aspect of the disclosure is a health information service providing device for a mobility user including an acquisition device, an estimation part, a calculation part, a current VLF data acquisition part, a past VLF data acquisition part, a state estimation part, and a service providing part. The acquisition device acquires a biological signal of the mobility user riding in a mobility. The estimation part estimates a heart rate signal related to a heart rate of the mobility user based on the biological signal. The calculation part calculates VLF data related to a very-low frequency component of 0.0033 to 0.04 Hz based on the heart rate signal. The current VLF data acquisition part acquires current VLF data, which is the VLF data of the mobility user this time including a current riding in a case of currently riding in the mobility or a most recent riding in a case of currently not riding in the mobility. The past VLF data acquisition part acquires past VLF data, which is a statistical representative value that is a numerical representative value or a positional representative value of the VLF data in a specific period in the past prior to this time. The state estimation part estimates a change in a health state of the mobility user based on a difference between the current VLF data and the past VLF data. The service providing part provides a specific service to the mobility user based on the change in the health state of the mobility user.

In addition, another aspect of the disclosure is a health information service providing device for a rider including an acquisition device, an estimation part, a calculation part, a current drowsiness sign data acquisition part, a past drowsiness sign data acquisition part, a state estimation part, and a service providing part. The acquisition device acquires a biological signal, which is a biological signal during riding, of a rider riding in a mobility. The estimation part estimates a heart rate signal related to a heart rate of the rider based on the biological signal. The calculation part calculates drowsiness sign data based on the heart rate signal, the drowsiness sign data being data related to a drowsiness sign of the rider and being a detection count of the drowsiness sign per unit time measured within a predetermined measurement time or a correlation value of the detection count. The current drowsiness sign data acquisition part acquires current drowsiness sign data, which is the drowsiness sign data of the rider in a state of currently riding in the mobility. The past drowsiness sign data acquisition part acquires past drowsiness sign data, which is the drowsiness sign data detected when the rider rode in the mobility in the past. The state estimation part estimates whether drowsiness of the rider has increased based on whether the current drowsiness sign data and the past drowsiness sign data satisfy a predetermined comparison condition. The service providing part executes control on providing equipment for providing a specific service to the rider, based on an estimation that the drowsiness of the rider has increased.

According to an aspect of the disclosure, based on VLF data obtained from the mobility user riding in the mobility, it is possible to estimate a change in the health state of the mobility user by relatively comparing the current VLF data during riding this time with the past VLF data during riding in the past. Based on the change in the health state estimated in this manner, it is possible to provide a service related to the health state to the mobility user.

In addition, according to another aspect of the disclosure, since whether the rider's drowsiness has increased is estimated based on whether the current drowsiness sign data and the past drowsiness sign data satisfy the comparison condition, it is possible to observe an over-time change in the rider's drowsiness. Accordingly, compared to the case of performing determination based solely on the current drowsiness sign data, which is the information of the rider of the present time, the accuracy of estimating the change in the rider's drowsiness can be improved.

Reference signs in parentheses described in the claims indicate correspondences with specific means described in the embodiments to be described later, and do not limit the technical scope of the disclosure.

### BRIEF DESCRIPTION OF THE DRAWINGS

FIG. 1 is a block diagram showing a health information service providing device of Embodiment 1.1.

FIG. 2 is a cross-sectional view showing a seat to which a sensor related to Embodiment 1.1 is assembled.

FIG. 3 is a graph showing a correlation between VLF data measured by the sensor and VLF measured by an electrocardiograph.

FIG. 4 is a view showing VLF data as statistical representative values in various periods.

FIG. 5 is a flowchart showing a main flow of the health information service providing device of Embodiment 1.1.

FIG. 6 is a flowchart showing a state estimation process 1 of Embodiment 1.1.

FIG. 7 is a flowchart showing a state estimation process 2 of Embodiment 1.1.

FIG. 8 is a flowchart showing a state estimation process 3 of Embodiment 1.1.

FIG. 9 is a flowchart showing a state estimation process 4 of Embodiment 1.1.

FIG. 10 is a flowchart showing a state estimation process 5 of Embodiment 1.1.

FIG. 11 is a block diagram showing a health information service providing device of Embodiment 1.2.

FIG. 12 is a flowchart showing a state estimation process 6 of Embodiment 1.2.

FIG. 13 is a flowchart showing a pre-determination process 1 of Embodiment 1.2.

FIG. 14 is a block diagram showing a health information service providing device of Embodiment 1.3.

FIG. 15 is a flowchart showing a state estimation process 7 of Embodiment 1.3.

FIG. 16 is a flowchart showing a state estimation process 8 of Embodiment 1.4.

FIG. 17 is a block diagram showing a health information service providing device of Embodiment 1.5.

FIG. 18 is a flowchart showing a state estimation process 9 of Embodiment 1.5.

FIG. 19 is a flowchart showing a state estimation process 10 of Embodiment 1.6.

FIG. 20 is a block diagram showing a health information service providing device of Embodiment 1.7.

FIG. 21 is a flowchart showing a state estimation process 11 of Embodiment 1.7.

FIG. 22 is a block diagram showing a health information service providing device of Embodiment 1.8.

FIG. 23 is a flowchart showing a state estimation process 12 of Embodiment 1.8.

FIG. 24 is a block diagram showing a health information service providing device of Embodiment 1.9.

FIG. 25 is a flowchart showing a state estimation process 13 of Embodiment 1.9.

FIG. 26 is a flowchart showing a pre-determination process 2 of Embodiment 1.9.

FIG. 27 is a cross-sectional view showing an embodiment in which a sensor related to another embodiment (1.2) is attached to an upper surface of a seat surface part.

FIG. 28 is a block diagram showing a health information service providing device related to another embodiment (1.3).

FIG. 29 is a block diagram showing a health information service providing device related to another embodiment (1.4).

FIG. 30 is a block diagram showing a health information service providing device related to another embodiment (1.5).

FIG. 31 is a block diagram showing a health information service providing device related to another embodiment (1.6).

FIG. 32 is a block diagram showing a health information service providing device related to another embodiment (1.7).

FIG. 33 is a block diagram showing a health information service providing device related to another embodiment (1.8).

FIG. 34 is a block diagram showing a health information service providing device related to another embodiment (1.9).

FIG. 35 is a block diagram showing a health information service providing device related to another embodiment (1.10).

FIG. 36 is a block diagram showing a drowsiness information service providing device of Embodiment 2.1.

FIG. 37 is a cross-sectional view showing a seat to which a sensor of Embodiment 2.1 is attached.

FIG. 38 is a graph showing over-time changes in a heartbeat interval related to drowsiness sign data of Embodiment 2.1.

FIG. 39 is a flowchart of a main routine of the drowsiness information service providing device of Embodiment 2.1.

FIG. 40 is a flowchart showing a state estimation process 21 of Embodiment 2.1.

FIG. 41 is a block diagram showing a drowsiness information service providing device of Embodiment 2.2.

FIG. 42 is a flowchart showing a state estimation process 22 of Embodiment 2.2.

FIG. 43 is a block diagram showing a drowsiness information service providing device of Embodiment 2.3.

FIG. 44 is a flowchart showing a state estimation process 23 of Embodiment 2.3.

FIG. 45 is a block diagram showing a drowsiness information service providing device of Embodiment 2.4.

FIG. 46 is a flowchart showing a state estimation process 24 of Embodiment 2.4.

FIG. 47 is a flowchart showing a state estimation process 25 of Embodiment 2.5.

FIG. 48 is a flowchart showing a state estimation process 26 of Embodiment 2.6.

FIG. 49 is a flowchart showing a state estimation process 27 of Embodiment 2.7.

FIG. 50 is a cross-sectional view showing a seat related to a drowsiness information service providing device of another embodiment (2.2).

FIG. 51 is a block diagram showing a drowsiness information service providing device of another embodiment (2.3).

FIG. 52 is a block diagram showing a drowsiness information service providing device of another embodiment (2.4).

FIG. 53 is a block diagram showing a drowsiness information service providing device of another embodiment (2.5).

FIG. 54 is a block diagram showing a drowsiness information service providing device of another embodiment (2.6).

FIG. 55 is a block diagram showing a drowsiness information service providing device of another embodiment (2.7).

FIG. 56 is a block diagram showing a drowsiness information service providing device of another embodiment (2.8).

FIG. 57 is a block diagram showing a drowsiness information service providing device of another embodiment (2.9).

FIG. 58 is a block diagram showing a drowsiness information service providing device of another embodiment (2.10).

### DESCRIPTION OF THE EMBODIMENTS

### Embodiment 1.1

### 1. Overview of Health Information Service Providing Device 10

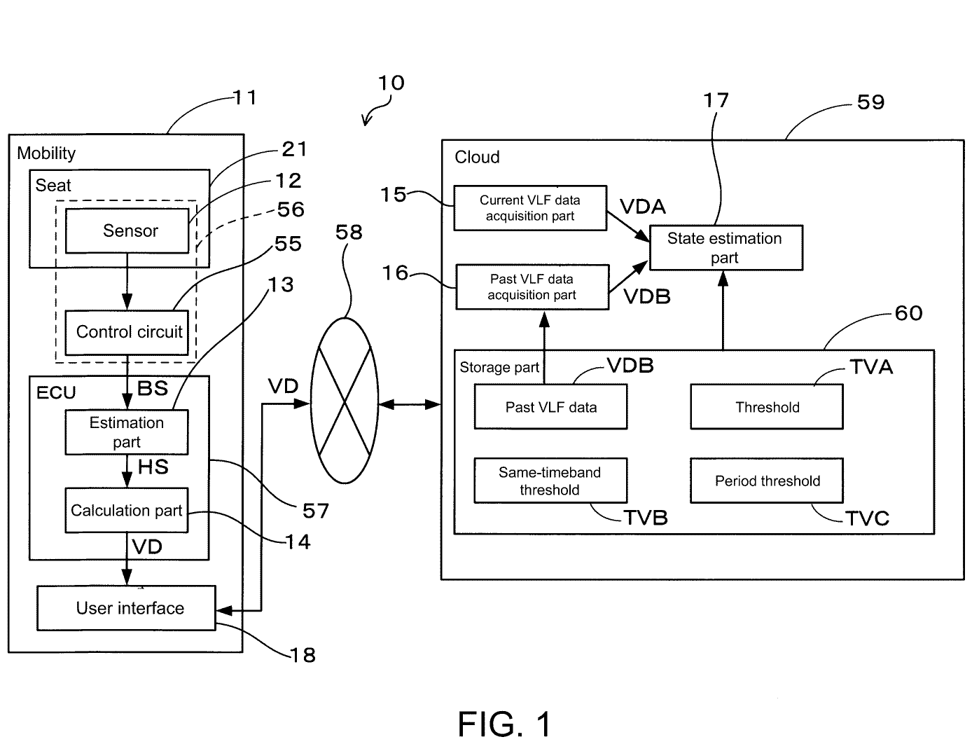

Referring to FIG. 1, an overview of a health information service providing device 10 of Embodiment 1.1 will be described. The health information service providing device 10 provides health information services to a mobility user who uses a mobility 11 according to changes in a health state of the mobility user.

The mobility 11 in this embodiment refers to a means of transportation that a person rides, including a vehicle such as a passenger vehicle, a cargo vehicle, a work vehicle, a train, a ship, an airplane, a helicopter, a passenger drone, etc. The mobility user in this embodiment includes a person who rides in and drives a mobility 11, a person who rides in an autonomously driven mobility 11, or a person who rides in a mobility 11 driven by another person.

As shown in FIG. 1, the health information service providing device 10 of this embodiment includes a sensor 12, an estimation part 13, a calculation part 14, a current VLF data acquisition part 15, a past VLF data acquisition part 16, a state estimation part 17, and a user interface 18 (an example of a service providing part).

The mobility 11 includes a seat 21 where the mobility user sits. The sensor 12 is attached to the seat 21. In the case where the seat 21 is applied to a driver's seat, the seat 21 is used to detect a seated state of the driver and a biological signal BS of the driver during driving. In the case where the seat 21 is applied to a seat different from the driver's seat, the seat 21 is used to detect a seated state and a biological signal BS of a rider of the mobility 11 who is different from the driver.

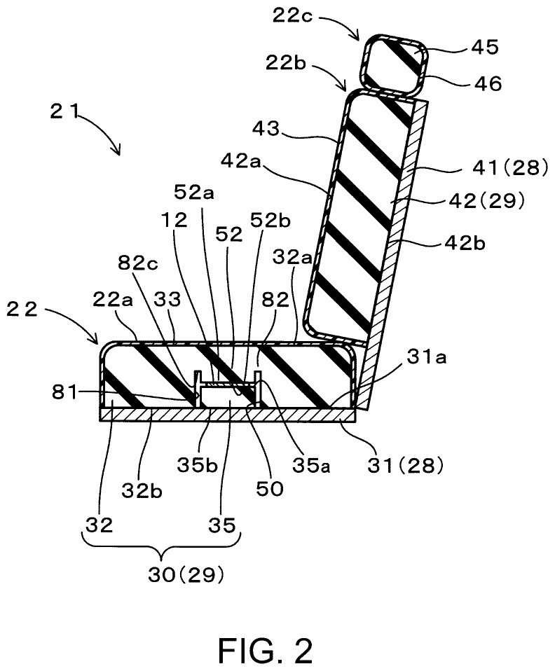

As shown in FIG. 2, the seat 21 includes a frame part 28, a cushion part 29, and the sensor 12. The seat 21 of this embodiment includes a headrest 22c. However, the headrest 22c may also be omitted.

The frame part 28 includes a seat-surface seat frame 31 and a back-surface seat frame 41. The cushion part 29 includes a seat-surface seat cushion 30 attached to the seat-surface seat frame 31 and a back-surface seat cushion 42 attached to the back-surface seat frame 41. The seat-surface seat cushion 30 includes a first seat-surface seat cushion 32 and a second seat-surface seat cushion 35.

The seat-surface seat frame 31 is formed of a rigid material such as metal or hard resin, for example, and is attached to a vehicle. The seat-surface seat frame 31 has a plate-shaped part forming a plate shape. The plate-shaped part is attached to the vehicle such that a plate surface thereof faces an up-down direction. An upper surface of the plate-shaped part is taken as an attachment seat surface 31a to which the first seat-surface seat cushion 32 is attached. Portions of the seat-surface seat frame 31 other than the plate-shaped part are formed in any shape such as a rod shape, a pillar shape, etc.

The first seat-surface seat cushion 32 is formed of an elastic material such as foam resin. The first seat-surface seat cushion 32 is attached in a state of being placed on the attachment seat surface 31a formed on the upper surface of the seat-surface seat frame 31. An upper surface of the first seat-surface seat cushion 32 serves as a pressure-receiving surface 32a that receives pressure from the rider's buttocks. A lower surface of the first seat-surface seat cushion 32, i.e., a counter-pressure-receiving surface 32b which is a surface on a back side of the pressure-receiving surface 32a, is opposed to the attachment seat surface 31a of the seat-surface seat frame 31.

The first seat-surface seat cushion 32 has an accommodating recess 50 that is opened downward toward the seat-surface seat frame 31 side on the counter-pressure-receiving surface 32b. A cross-sectional shape of the accommodating recess 50 may be any shape such as a polygonal shape, a circular shape, an elliptical shape, etc. In this embodiment, the cross-sectional shape of the accommodating recess 50 is rectangular. A portion of the accommodating recess 50 that is located on a side opposite to an opening direction of the accommodating recess 50 is taken as a bottom part 52 of the accommodating recess 50.

The accommodating recess 50 accommodates the second seat-surface seat cushion 35. The second seat-surface seat cushion 35 is attached to the attachment seat surface 31a of the seat-surface seat frame 31. A surface of the second seat-surface seat cushion 35 that is opposed to the attachment seat surface 31a of the seat-surface seat frame 31 is taken as an attachment surface 35b. With the first seat-surface seat cushion 32 and the second seat-surface seat cushion 35 attached to the seat-surface seat frame 31, the counter-pressure-receiving surface 32b of the first seat-surface seat cushion 32 and the attachment surface 35b of the second seat-surface seat cushion 35 are flush with each other. In addition, with the first seat-surface seat cushion 32 and the second seat-surface seat cushion 35 attached to the seat-surface seat frame 31, a gap is formed between an inner surface of the accommodating recess 50 and an outer surface of the second seat-surface seat cushion 35. An upper surface (a surface located on a side opposite to the seat-surface seat frame 31) of the second seat-surface seat cushion 35 is taken as a second pressing surface 35a that presses the sensor 12 from below. However, the accommodating recess 50 of the first seat-surface seat cushion 32 may also be configured to be opened upward, and the second seat-surface seat cushion 35 may also be configured to be accommodated in such an accommodating recess 50.

The surface of the first seat-surface seat cushion 32 is covered with a seat-surface skin member 33. The seat-surface skin member 33 covers at least the pressure-receiving surface 32a of the first seat-surface seat cushion 32. The seat-surface skin member 33 is formed of a material that is less stretchable than the first seat-surface seat cushion 32, such as fabric or leather.

The back-surface seat frame 41 is formed of a rigid material such as metal or hard resin, for example. The back-surface seat frame 41 is formed in a plate shape, a rod shape, etc. For example, in the case of providing a reclining function to a seat body 22, the back-surface seat frame 41 is swingably supported at the seat-surface seat frame 31. Of course, the back-surface seat frame 41 may also be integrally fixed to the seat-surface seat frame 31.

The back-surface seat cushion 42 is formed of an elastic material such as foam resin. The back-surface seat cushion 42 is layered and attached to the back-surface seat frame 41. A surface of the back-surface seat cushion 42 on a side opposite to the back-surface seat frame 41 serves as a surface that receives pressure from the rider's back. In other words, a surface of the back-surface seat cushion 42 that is opposed to the back-surface seat frame 41 serves as a counter-pressure-receiving surface 42b of the back-surface seat cushion 42.

The surface of the back-surface seat cushion 42 is covered with a back-surface skin member 43. The back-surface skin member 43 covers at least a pressure-receiving surface 42a of the back-surface seat cushion 42. The back-surface skin member 43 is formed of a material such as fabric or leather.

The headrest 22c is disposed at an upper end of a seat back part 22b. The headrest 22c includes a cushion 45 and a skin member 46. Herein, in FIG. 2, the first seat-surface seat cushion 32 and the back-surface seat cushion 42 are separate from each other, but the first seat-surface seat cushion 32 and the back-surface seat cushion 42 may also be integrated with each other. In addition, the back-surface seat cushion 42 and the headrest 22c are separate from each other, but the back-surface seat cushion 42 and the headrest 22c may also be integrated with each other.

The sensor 12 is disposed between a first pressing surface 52b formed in the accommodating recess 50 of the first seat-surface seat cushion 32 and the second pressing surface 35a of the second seat-surface seat cushion 35. The first pressing surface 52b is provided on a protruding part 52a that protrudes downward. Herein, with the rider sitting on the seat body 22, a pressure is applied from the rider's buttocks to the pressure-receiving surface 32a of the first seat-surface seat cushion 32, and this pressure is transmitted via the first seat-surface seat cushion 32 to the first pressing surface 52b of the first seat-surface seat cushion 32. Then, the sensor 12 receives a pressure from the first pressing surface 52b of the first seat-surface seat cushion 32. In other words, in the seated state of the rider, the sensor 12 detects a physical amount corresponding to the pressure transmitted via the first seat-surface seat cushion 32 from the pressure-receiving surface 32a of the first seat-surface seat cushion 32.

Herein, the sensor 12 is disposed in a seat surface part 22a, but the sensor 12 may also be disposed in the seat back part 22b. In that case, the sensor 12 is disposed between the back-surface seat frame 41 and the back-surface seat cushion 42. Then, in the seated state of the rider, the sensor 12 detects a physical amount corresponding to a pressure transmitted via the back-surface seat cushion 42 from the pressure-receiving surface 42a of the back-surface seat cushion 42.

The sensor 12 is connected to a control circuit 55 for controlling the sensor 12. The control circuit 55 controls an action of the sensor 12 and acquires the physical amount detected by the sensor 12 from the sensor 12. The control circuit 55 detects a biological signal BS of the mobility user based on the physical amount acquired from the sensor 12. The control circuit 55 may perform processing such as amplification and noise cancellation on the biological signal BS.

The sensor 12 and the control circuit 55 constitute a monitoring system 56 (an example of an acquisition device) that monitors the biological signal BS of the mobility user.

The control circuit 55 transmits the biological signal BS to an electronic control unit (ECU) 57. The ECU 57 includes an estimation part 13 and a calculation part 14.

The estimation part 13 acquires the biological signal BS acquired from the control circuit 55 of the monitoring system 56. The biological signal BS is a generic concept of a heart rate, a body movement, a respiration, etc. of the mobility user, and also includes a signal in which signals of the heart rate, the body movement, the respiration, etc. are mixed. A pulse rate refers to pulsation occurring in arteries. On the other hand, the heart rate refers to pulsation of the heart pumping blood throughout the body. In a healthy individual, since the heart rate and the pulse rate generally coincide with each other, hereinafter, the heart rate and the pulse rate will be collectively referred to as a heart rate signal HS related to the heart rate. The estimation part 13 estimates a heart rate signal HS related to the heart rate of the mobility user from the biological signal BS.

The calculation part 14 calculates VLF data VD related to a very-low frequency component of 0.0033 to 0.04 Hz. As described above, conventionally, VLF has been used in medical institutions as an indicator of a person's health state. The VLF is calculated from an electrocardiogram measured by an electrocardiograph mounted to a person. In the electrocardiogram obtained from the electrocardiograph, a peak of the heart rate is clearly presented, so the VLF can be calculated from the peak.

However, in this embodiment, the sensor 12 receives a pressure from the buttocks of the mobility user seated on the seat 21, detects a biological signal BS from this pressure, and estimates a heart rate signal HS from this biological signal BS. Thus, the data related to the very-low frequency component obtained based on the heart rate signal HS in this embodiment is, strictly speaking, different from the VLF obtained from an electrocardiogram. Accordingly, hereinafter, the data related to the very-low frequency component obtained based on the heart rate signal HS in this embodiment will be referred to as VLF data VD to distinguish from the VLF obtained from an electrocardiogram.

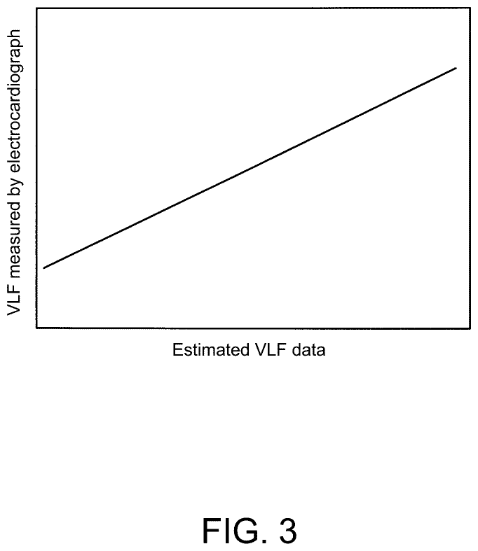

As shown in FIG. 3, it is learned that the VLF data VD according to this embodiment and the VLF obtained from an electrocardiogram are in a highly correlated relationship. Thus, the VLF data VD obtained from the mobility user seated on the seat 21 may be used to estimate changes in the health state of the mobility user.

The calculation part 14 transmits the VLF data VD to the user interface 18. Examples of the user interface 18 include a car navigation system, a smartphone, a tablet terminal, a smartwatch, etc. In this embodiment, a car navigation system installed in the mobility 11 is adopted.

The user interface 18 includes both or one of a screen display device and a speaker for providing specific services to the mobility user. The user interface 18 provides specific services to the mobility user by an image or a voice.

The user interface 18 includes a communication device (not shown), and is connected to a network 58 such as the Internet by the communication device. The user interface 18 transmits and receives data to and from a cloud 59 via the network 58.

The cloud 59 includes a current VLF data acquisition part 15, a past VLF data acquisition part 16, a state estimation part 17, and a storage part 60.

The current VLF data acquisition part 15 acquires current VLF data VDA from the user interface 18 via the network 58. The current VLF data VDA is VLF data VD of the mobility user this time during current riding in the case where the mobility user is currently riding in the mobility 11, and is VLF data VD of the mobility user including a most recent riding in the case where the mobility user is currently not riding in the mobility 11.

In the case where the mobility user is currently riding in the mobility 11, a statistical representative value of VLF data VD in a period including at least a part of the current riding is taken as the current VLF data VDA. In addition, in the case where the mobility user is currently not riding in the mobility 11, a statistical representative value of VLF data VD in a period including at least a part of the most recent riding is taken as the current VLF data VDA.

The VLF data VD transmitted from the user interface 18 to the cloud 59 via the network 58 is acquired by the current VLF data acquisition part 15 and is sequentially stored in the storage part 60 as past VLF data VDB.

The past VLF data acquisition part 16 acquires past VLF data VDB stored in the storage part 60. The past VLF data VDB is a statistical representative value of VLF data VD in a specific period in the past. The statistical representative value includes a numerical representative value or a positional representative value.

A statistical representative value is a characteristic value that numerically summarizes a central tendency of a frequency distribution when calculating statistical data. The statistical representative value represents information included in a set of data by one numerical value, and includes two types, i.e., a numerical representative value and a positional representative value.

The numerical representative value, also referred to as a mathematical representative value, is classified into an arithmetic mean, a geometric mean, a harmonic mean, a root mean square, etc. The arithmetic mean, also referred to as an arithmetic average, is a value obtained by dividing a sum of data by a quantity of the data. The geometric mean, also referred to as a product mean, is an Nth root of a product of individual data. The harmonic mean refers to a reciprocal of an arithmetic mean of reciprocals of each data. The root mean square is a square root of an arithmetic mean of squared values of data.

The positional representative value is further divided into a median, a mode, quantiles, etc. These values are obtained by regarding one or a few data occupying specific positions in a frequency distribution as representative values of all data. The median is a value located exactly in the middle when measured values are arranged in an ascending order. The mode refers to a value that appears most frequently or exists most commonly in a set of data. The quantiles are derived from the same concept as the median, and include quartiles, quintiles, deciles, percentiles, etc., which divide the data at various points. For example, in terms of quartiles, values of data located at ¼, 2/4, and ¾ positions in an entire data series arranged in an ascending order are respectively called a first quartile, a second quartile, and a third quartile.

The state estimation part 17 estimates changes in the health state of the mobility user based on a difference between the current VLF data VDA and the past VLF data VDB.

The storage part 60 stores the past VLF data VDB transmitted from the user interface 18 via the network 58. In addition, the storage part 60 stores a threshold TVA, a same-timeband threshold TVB, and a period threshold TVC.

### 2. Regarding VLF Data VD

### 2.1. Regarding Statistical Representative Value

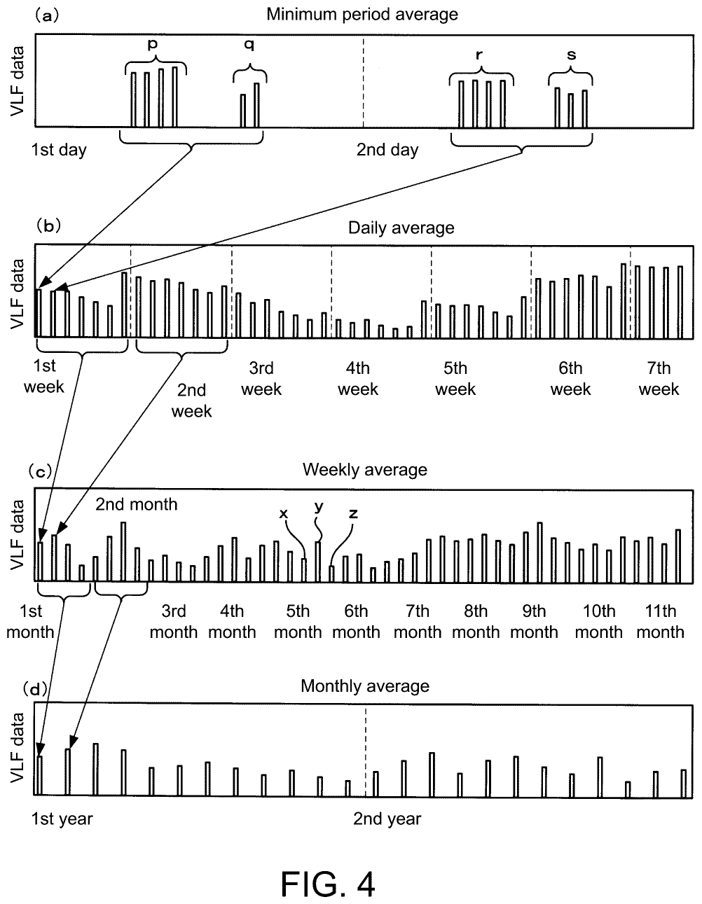

Next, referring to FIG. 4, the VLF data VD will be described. When a mobility rider rides in the mobility 11 and sits on the seat 21, the monitoring system 56 (sensor 12 and control circuit 55) acquires a biological signal BS of the mobility rider. Based on the biological signal BS, the estimation part 13 estimates a heart rate signal HS, and the calculation part 14 calculates VLF data VD. The VLF data VD is calculated as a statistical representative value related to a very-low frequency component in a predetermined period. In this case, the VLF data VD is calculated as an arithmetic mean value of a time of data related to the very-low frequency component in the predetermined period.

(a) of FIG. 4 shows results of measuring VLF data VD of a mobility user over two days. The vertical axis represents an intensity of the VLF data VD. The larger the VLF data VD is, the better state the health state of the mobility user can be determined. In contrast, if the VLF data VD becomes small, it can be determined that the health state of the mobility user is unfavorable. The horizontal axis represents elapsed time.

A width of each bar graph shown in (a) of FIG. 4 indicates a minimum period for calculating the VLF data VD as a representative value. The minimum period may be set to any value in a range of several minutes to several tens of minutes.

In (a) of FIG. 4, four bar graphs labeled with a symbol “p” represent VLF data VD when the mobility user rides in the mobility 11 in the morning of a 1st day of measurement. At this time, the mobility user rides in the mobility 11 for a duration of four minimum periods.

Two bar graphs labeled with a symbol “q” represent VLF data VD when the mobility user rides in the mobility 11 in the afternoon of the 1st day of measurement. At this time, the mobility user rides in the mobility 11 for a duration of two minimum periods.

Four bar graphs labeled with a symbol “r” represent VLF data VD when the mobility user rides in the mobility 11 in the morning of a 2nd day of measurement. At this time, the mobility user rides in the mobility 11 for a duration of four minimum periods.

A timeband in which the VLF data VD of the 2nd day labeled with the symbol “r” is measured is almost the same as a timeband in which the VLF data VD of the 1st day labeled with the symbol “p” is measured. In other words, the timeband in which the VLF data VD of the 2nd day labeled with the symbol “r” is measured includes the same time point as the time point at which the VLF data VD of the 1st day labeled with the symbol “p” is measured.

Two bar graphs labeled with a symbol “s” represent VLF data VD when the mobility user rides in the mobility 11 in the afternoon of the 2nd day of measurement. At this time, the mobility user rides in the mobility 11 for a duration of three minimum periods.

A timeband in which the VLF data VD of the 2nd day labeled with the symbol “s” is measured is almost the same as a timeband in which the VLF data VD of the 1st day labeled with the symbol “q” is measured. In other words, the timeband in which the VLF data VD of the 2nd day labeled with the symbol “s” is measured includes the same time point as the time point at which the VLF data VD of the 1st day labeled with the symbol “q” is measured.

In (b) FIG. 4, one bar graph represents an average value of the VLF data VD for one day. A leftmost bar graph in (b) of FIG. 4 is a daily average of the VLF data VD of the 1st day in (a) of FIG. 4. A second bar graph from the left in (b) of FIG. 4 is a daily average of the VLF data VD of the 2nd day in (a) of FIG. 4. Subsequently, in (b) of FIG. 4, daily averages of VLF of a 3rd day and a 4th day are shown from left to right. In (b) of FIG. 4, dashed lines represent divisions of weeks. (b) of FIG. 4 shows changes in the VLF data VD up to a 7th week since the start of measuring the health state of the mobility user.

In (c) of FIG. 4, one bar graph represents an average value of the VLF data VD for one week. A leftmost bar graph in (c) of FIG. 4 is a weekly average of the VLF data VD of a 1st week in (b) of FIG. 4. A second bar graph from the left in (c) of FIG. 4 is a weekly average of the VLF data VD of a 2nd week in (b) of FIG. 4. Subsequently, in (c) of FIG. 4, daily averages of VLF of a 3rd week and a 4th week are shown from left to right. (c) of FIG. 4 shows changes in the VLF data VD up to an 11th month since the start of measuring the health state of the mobility user. Since divisions of weeks and divisions of months do not necessarily coincide, (c) of FIG. 4 does not show divisions of months.

In (d) of FIG. 4, one bar graph represents an average value of the VLF data VD for one month. A leftmost bar graph in (d) of FIG. 4 is a monthly average of the VLF data VD of a 1st month in (c) FIG. 4. A second bar graph from the left in (d) of FIG. 4 is a monthly average of the VLF data VD of a 2nd month in (c) of FIG. 4. Subsequently, in (d) of FIG. 4, monthly averages of VLF of a 3rd month and a 4th month are shown from left to right. In (d) of FIG. 4, a dashed line represents a division between a 1st year and a 2nd year. (d) of FIG. 4 shows changes in the VLF data VD up to the 2nd year since the start of measuring the health state of the mobility user.

### 2.2. Regarding Relative Comparison

As shown in (a) of FIG. 4, this embodiment is configured such that the VLF data VD is acquired when the mobility user rides in the mobility 11. Accordingly, the VLF data VD is acquired under relatively similar conditions in daily life. By performing a relative comparison among multiple VLF data VD acquired under relatively similar conditions in such a manner, it is possible to estimate changes in the health state of the mobility rider.

For example, the bar graphs labeled with symbols “x”, “y”, and “z” in (c) of FIG. 4 will be examined. As described above, (c) of FIG. 4 shows the weekly averages of the VLF data VD. Comparing the VLF data VD of an x-th week labeled with the symbol “x” and the VLF data VD of a y-th week labeled with the symbol “y”, the VLF data VD of the y-th week is larger than the VLF data VD of the x-th week. Accordingly, it is estimated that the health state of the mobility user has changed in a favorable direction in the y-th week compared to the x-th week. In contrast, the VLF data VD of a z-th week is smaller compared to the VLF data VD of the y-th week. Accordingly, it is estimated that the health state of the mobility user has changed in an unfavorable direction in the z-th week compared to the y-th week.

### 3. Action of Embodiment

### 3.1. Main Routine

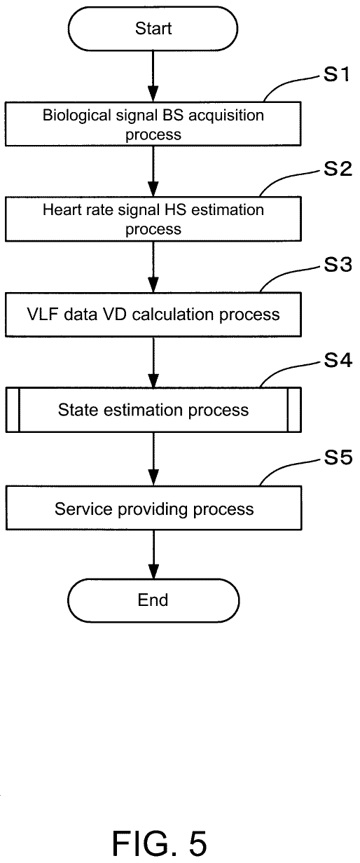

Next, referring to FIG. 5, an action of the health information service providing device 10 of this embodiment will be described. FIG. 5 shows a flowchart of the action of the health information service providing device 10 of this embodiment. Upon activation of the health information service providing device 10, a biological signal acquisition process S1 is executed. In the biological signal acquisition process S1, the monitoring system 56 (sensor 12 and control circuit 55) acquires a biological signal BS from the mobility user seated on the seat 21 of the mobility 11. The biological signal BS is transmitted to the control circuit 55.

Next, a heart rate signal estimation process S2 is executed. In the heart rate signal estimation process S2, the estimation part 13 estimates a heart rate signal HS related to the heart rate of the mobility user based on the biological signal BS acquired from the monitoring system 56.

Next, a VLF data calculation process S3 is executed. In the VLF data calculation process S3, the calculation part 14 calculates VLF data VD based on the heart rate signal HS acquired from the estimation part 13. The calculated VLF data VD is transmitted to the cloud 59 via the network 58 from the user interface 18.

Next, a state estimation process S4 is executed. In the state estimation process S4, the state estimation part 17 estimates a change in the health state of the mobility user based on a difference between current VLF data VDA and past VLF data VDB.

Next, a service providing process S5 is executed. In the service providing process S5, the service providing part provides a specific service to the mobility user based on information related to the change in the health state of the mobility user acquired from the state estimation part 17.

Upon ending of the service providing process S5, the action of the health information service providing device 10 is ended.

### 3.2. State Estimation Process

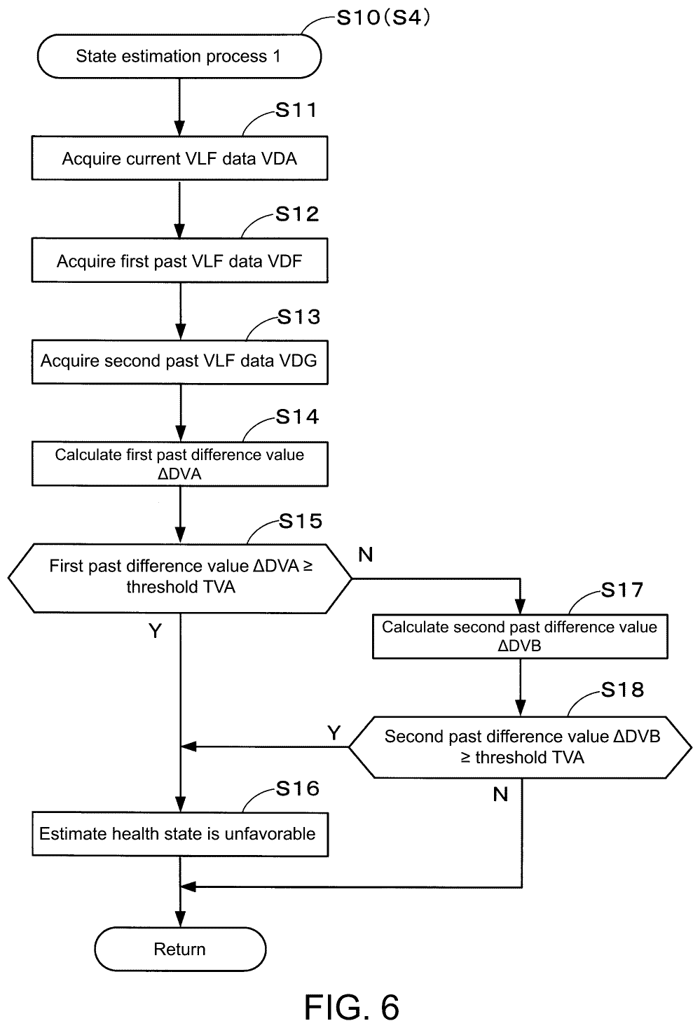

Next, referring to FIG. 6 to FIG. 10, the state estimation process S4 will be described. In this embodiment, as shown in FIG. 6 to FIG. 10, the state estimation process S4 includes five processes, i.e., a state estimation process 1 (S10) to a state estimation process 5 (S50). All of the state estimation process 1 (S10) to the state estimation process 5 (S50) may be executed, or one or more selected from the state estimation process 1 (S10) to the state estimation process 5 (S50) may be executed. In the case of selecting from the state estimation process 1 (S10) to the state estimation process 5 (S50), the mobility user may select one or more from the five processes, or the processes may be selected in advance according to specifications or a destination of the health information service providing device 10. Hereinafter, the state estimation process 1 (S10) to the state estimation process 5 (S50) will be described. However, the state estimation process S4 may also be configured to include one to four, or six or more state estimation processes.

### (1) State Estimation Process 1

FIG. 6 shows a flowchart of the state estimation process 1 (S10). As will be described in detail later, the state estimation process 1 of this embodiment determines a first past difference value ΔDVA using a threshold TVA, and also determines a second past difference value ΔDVB using the threshold TVA.

Upon execution of the state estimation process 1 (S10), the current VLF data acquisition part 15 acquires current VLF data VDA (S11).

Next, the past VLF data acquisition part 16 acquires first past VLF data VDF from the storage part 60 (S13). The first past VLF data VDF is a statistical representative value of VLF data VD in a specific period that is one or more times prior to the specific period in which the current VLF data VDA is acquired. Any statistical representative value may be appropriately selected as the statistical representative value, such as an arithmetic mean value of VLF data VD during one-time riding, a daily average value of VLF data VD, a weekly average value of VLF data VD, a monthly average value of VLF data VD, etc.

Next, the past VLF data acquisition part 16 acquires second past VLF data VDG from the storage part 60 (S13). The second past VLF data VDG is a statistical representative value of VLF data VD in a specific period that is one or more times prior to the period in which the first past VLF data VDF is acquired. Similar to the first past VLF data VDF, any statistical representative value may be appropriately selected as the statistical representative value, such as an arithmetic mean value of VLF data VD during one-time riding, a daily average value of VLF data VD, a weekly average value of VLF data VD, a monthly average value of VLF data VD, etc. In this embodiment, the period of the first past VLF data VDF and the period of the second past VLF data VDG are set to be the same. For example, if the first past VLF data VDF is a daily average value, the second past VLF data VDG is also taken as a daily average value.

Next, the state estimation part 17 calculates a first past difference value ΔDVA obtained by subtracting the current VLF data VDA from the first past VLF data VDF (S14). In other words, the state estimation part 17 examines how a current health state of the mobility user has changed compared to a past health state. Then, the state estimation part 17 determines whether the first past difference value ΔDVA is equal to or greater than the threshold TVA stored in the storage part 60 (S15). Herein, the threshold TVA is an example of a first comparison criterion.

In the case where the first past difference value ΔDVA is equal to or greater than the threshold TVA (S15: Y), it means that the VLF data VD related to the current health state of the mobility user has decreased compared to the VLF data VD related to the past health state. Thus, the state estimation part 17 determines that the health state of the mobility user has changed in an unfavorable direction (S16). Accordingly, the state estimation process 1 (S10) is ended.

In contrast, in the case where the first past difference value ΔDVA is smaller than the threshold TVA (S15: N), the state estimation part 17 calculates a second past difference value ΔDVB obtained by subtracting the current VLF data VDA from the second past VLF data VDG (S17). In other words, the state estimation part 17 examines how the current health state of the mobility user has changed compared to a health state in the further past than the period in which the first past VLF data VDF is measured. Then, the state estimation part 17 determines whether the second past difference value ΔDVB is equal to or greater than the threshold TVA stored in the storage part 60 (S18). Herein, the threshold TVA is an example of a second comparison criterion.

In the case where the second past difference value ΔDVB is equal to or greater than the threshold TVA (S18: Y), it means that the VLF data VD related to the current health state of the mobility user has decreased compared to the VLF data VD related to the health state in the further past than the period in which the first past VLF data VDF is measured. Thus, the state estimation part 17 determines that the health state of the mobility user has changed in an unfavorable direction (S16). Accordingly, the state estimation process 1 (S10) is ended.

In the case where the second past difference value ΔDVB is smaller than the threshold TVA (S18: N), the state estimation process 1 (S10) is ended.

### (2) State Estimation Process 2

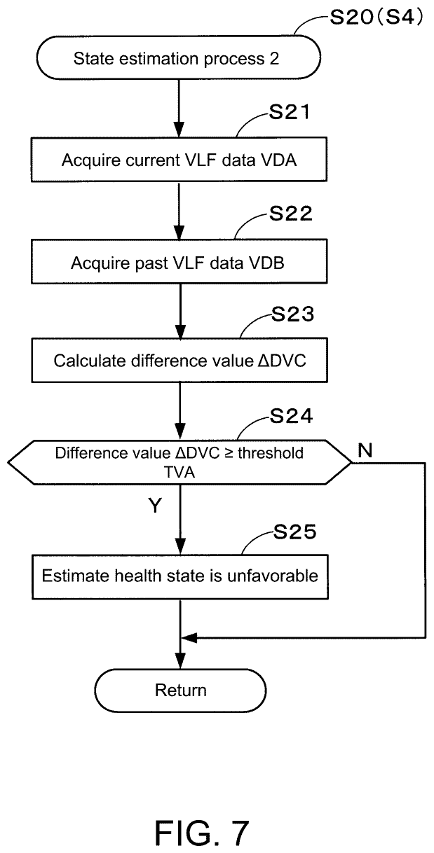

FIG. 7 shows a flowchart of the state estimation process 2 (S20). As will be described in detail later, the state estimation process 2 (S20) of this embodiment is an embodiment that compares current VLF data VDA of one-time riding with past VLF data VDB of multiple-time ridings in the past. Upon execution of the state estimation process 2 (S20), the current VLF data acquisition part 15 acquires current VLF data VDA of one-time riding, which is a current riding or a most recent riding (S21).

Next, the past VLF data acquisition part 16 acquires, from the storage part 60, past VLF data VDB, which is a statistical representative value of VLF data VD of multiple-time ridings in a predetermined period in the past (S22). A statistical representative value of multiple-time ridings may be appropriately selected as the statistical representative value, such as an arithmetic mean value of VLF data VD during multiple-time ridings, a daily average value of VLF data VD, a weekly average value of VLF data VD, a monthly average value of VLF data VD, etc.

Next, the state estimation part 17 calculates a difference value ΔDVC obtained by subtracting the current VLF data VDA from the past VLF data VDB (S23). In other words, the state estimation part 17 examines how a current health state of the mobility user has changed compared to a health state during multiple-time ridings of the mobility 11 in the past. Then, the state estimation part 17 determines whether the difference value ΔDVC is equal to or greater than the threshold TVA stored in the storage part 60 (S24).

In the case where the difference value ΔDVC is equal to or greater than the threshold TVA (S24: Y), it means that the current VLF data VDA related to the health state of the mobility user has decreased from the past VLF data VDB related to the health state during multiple-time ridings of the mobility 11 in the past. Thus, the state estimation part 17 determines that the health state of the mobility user has changed in an unfavorable direction (S25). Accordingly, the state estimation process 2 (S20) is ended.

In contrast, in the case where the difference value ΔDVC is smaller than the threshold TVA (S24: N), the state estimation process 2 (S20) is ended.

### (3) State Estimation Process 3

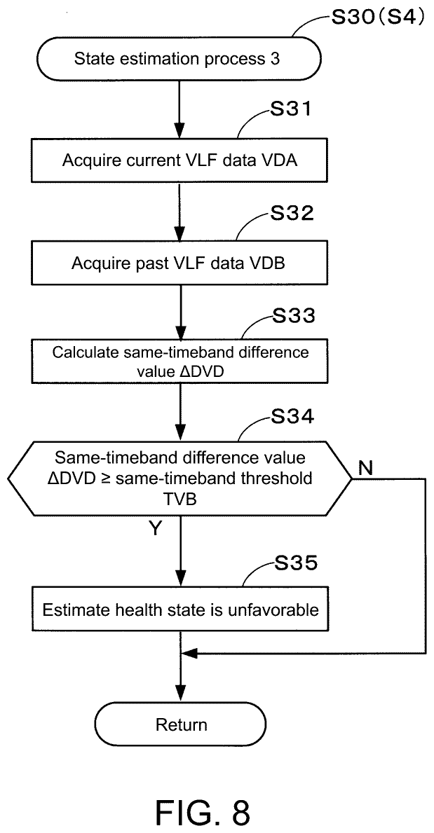

FIG. 8 shows a flowchart of the state estimation process 3 (S30). As will be described in detail later, the state estimation process 3 (S30) of this embodiment is an embodiment that compares current VLF data VDA with past VLF data VDB including a same time point as a time point at which the current VLF data VDA is measured. Upon execution of the state estimation process 3 (S30), the current VLF data acquisition part 15 acquires current VLF data VDA, which is VLF data VD during a current riding or a most recent riding (S31).

Next, the past VLF data acquisition part 16 acquires past VLF data VDB, which is a statistical representative value of VLF data VD during past riding including the same time point as the time point at which the current VLF data VDA is measured (S32).

Next, the state estimation part 17 calculates a same-timeband difference value ΔDVD obtained by subtracting the current VLF data VDA from the past VLF data VDB (S33). In other words, the state estimation part 17 examines how a current health state of the mobility user has changed compared to a past health state of the mobility user during riding in a same timeband. Then, the state estimation part 17 determines whether the same-timeband difference value ΔDVD is equal to or greater than the same-timeband threshold TVB stored in the storage part 60 (S34).

In the case where the same-timeband difference value ΔDVD is equal to or greater than the same-timeband threshold TVB (S34: Y), it means that the current VLF data VDA related to the health state of the mobility user has decreased from the past VLF data VDB related to the past health state during riding of the mobility 11 in the same timeband. Thus, the state estimation part 17 determines that the health state of the mobility user has changed in an unfavorable direction (S35). Accordingly, the state estimation process 3 (S30) is ended.

In contrast, in the case where the same-timeband difference value ΔDVD is smaller than the same-timeband threshold TVB (S34: N), the state estimation process 3 (S30) is ended.

### (4) State Estimation Process 4

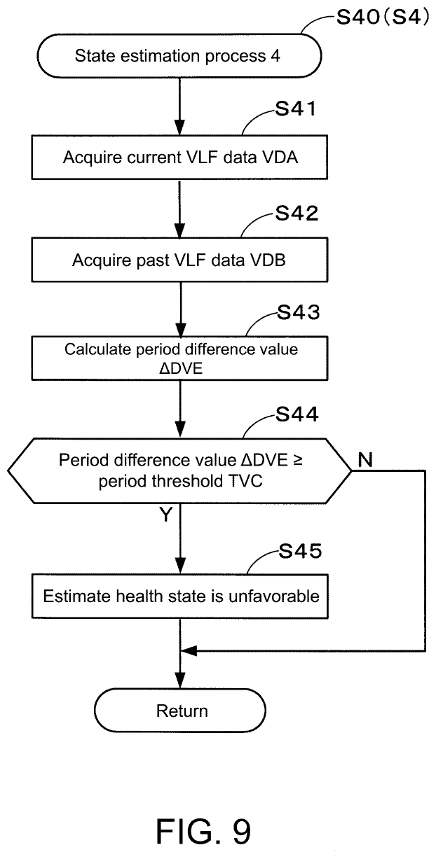

FIG. 9 shows a flowchart of the state estimation process 4 (S40). The state estimation process 4 (S40) of this embodiment is an embodiment that compares current VLF data VDA and past VLF data VDB in a predetermined comparison period including a time when the mobility user rides in the mobility 11. Upon execution of the state estimation process 4 (S40), the current VLF data acquisition part 15 acquires current VLF data VDA, which is a statistical representative value of VLF data VD in a predetermined comparison period including a current riding or a most recent riding (S41). Any period may be appropriately selected as the comparison period, such as any number of ridings including one time or two or more times of ridings, one day, one week, one month, etc., for example.

Next, the past VLF data acquisition part 16 acquires past VLF data VDB, which is a statistical representative value upon acquisition of VLF data VD in a comparison period prior to the current riding or the most recent riding (S42). The comparison period related to the past VLF data VDB may be the same as or different from the comparison period related to the current VLF data VDA.

Next, the state estimation part 17 calculates a period difference value ΔDVE obtained by subtracting the current VLF data VDA from the past VLF data VDB (S43). In other words, the state estimation part 17 examines how a health state of the mobility user within a current comparison period has changed compared to a health state of the mobility user within a past comparison period. Then, the state estimation part 17 determines whether the period difference value ΔDVE is equal to or greater than the period threshold TVC stored in the storage part 60 (S44).

In the case where the period difference value ΔDVE is equal to or greater than the period threshold TVC (S44: Y), it means that the current VLF data VDA related to the health state of the mobility user within the comparison period has decreased from the past VLF data VDB related to the health state of the mobility user within the past comparison period. Thus, the state estimation part 17 determines that the health state of the mobility user has changed in an unfavorable direction (S45). Accordingly, the state estimation process 4 (S40) is ended.

In contrast, in the case where the period difference value ΔDVE is smaller than the period threshold TVC (S44: N), the state estimation process 4 (S40) is ended.

### (5) State Estimation Process 5

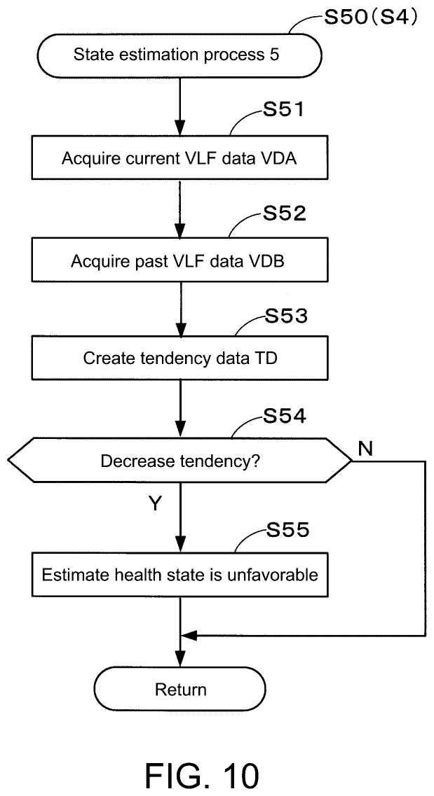

FIG. 10 shows a flowchart of the state estimation process 5 (S50). As will be described in detail later, the state estimation process 5 (S50) of this embodiment is an embodiment that estimates a change in the health state of the mobility user from a tendency of VLF data VD from a past riding to a current riding. Upon execution of the state estimation process 5 (S50), the current VLF data acquisition part 15 acquires current VLF data VDA (S51).

Next, the past VLF data acquisition part 16 acquires multiple past VLF data VDB (S52). From the viewpoint of lapse of time, the multiple past VLF data VDB may be continuous or may be discrete.

As an example of being continuous, the multiple past VLF data VDB may be, for example, continuous past VLF data VDB of one week counting back from the day on which the current VLF data VDA is acquired, or may be past VLF data VDB of one month counting back from the day on which the current VLF data VDA is acquired, and the period of the past VLF data VDB to be acquired is any period.

As an example of being discrete, the multiple past VLF data VDB may be multiple past VLF data VDB acquired on a same day of the week as the day on which the current VLF data VDA is acquired. For example, current VLF data VDA and past VLF data VDB of every Monday may be compared. In addition, current VLF data VDA and past VLF data VDB of the first day of every month may be compared. A time interval of the past VLF data VDB to be acquired is any time interval.

Next, the state estimation part 17 combines the multiple past VLF data VDB and the current VLF data VDA to create tendency data TD (S53). The tendency data TD is data in which the VLF data VD from the past to this time is compiled into one data, and it becomes possible to determine on a tendency of the VLF data VD from the past to this time.

Next, with respect to the tendency data TD, the state estimation part 17 determines whether most recent past tendency data TD of the time when the current VLF data VDA is acquired exhibits a decrease tendency (S54).

The tendency data TD may include, for example, a state in which the multiple past VLF data VDB monotonically decrease up to the current VLF data VDA. In that case, the most recent past tendency data TD of the time when the current VLF data VDA is acquired exhibits a decrease tendency. Thus, the state estimation part 17 determines that the most recent past tendency data TD of the time when the current VLF data VDA is acquired exhibits a decrease tendency (S54: Y).

In addition, the tendency data TD may include, for example, a state in which the multiple past VLF data VDB repeat cycles of decrease and increase up to the current VLF data VDA. In that case, the most recent past tendency data TD of the time when the current VLF data VDA is acquired is either in a decrease tendency or in an increase tendency. The state estimation part 17 determines that the tendency data TD is in a decrease tendency (S54: Y) in the case where the most recent past tendency data TD of the time when the current VLF data VDA is acquired is in a decrease tendency.

In the case where the tendency data TD is in a decrease tendency (S54: Y), it can be determined that the VLF data VD related to the health state of the mobility user is in a decrease tendency as a whole. Thus, the state estimation part 17 determines that the health state of the mobility user has changed in an unfavorable direction (S55). Accordingly, the state estimation process 5 (S50) is ended.

In contrast, in the case where the tendency data TD is not in a decrease tendency (S54: N), the state estimation process 5 (S50) is ended.

The tendency of the VLF data VD described above includes both or one of a state in which the multiple past VLF data VDB monotonically decrease up to the current VLF data VDA, and a state in which the multiple past VLF data VDB repeat cycles of decrease and increase up to the current VLF data VDA.

### 3.3. Service Providing Process S5

When the health state of the mobility user has changed in an unfavorable direction, the user interface 18 provides a specific service to the mobility user.

The specific service is not particularly limited, and for example, the following services may be provided. The service providing part may, for example, convey a message to the mobility user by a voice or by displaying on a screen, concisely informing that the health state has changed in an unfavorable direction. The message is not particularly limited, and may be any message such as “You seem to be tired lately.”, “How about taking a break?”, etc.

In addition, the user interface 18 may convey a message to the mobility user by a voice or by displaying on a screen, providing proposals for improving the health state. The message is not particularly limited, and may be any message such as “How about doing some sports?”, “How about going on a trip?”, etc.

In addition, the user interface 18 may communicate to the mobility user about facilities where recovery of the health state can be expected, and routes to such facilities. According to preferences of the mobility user, the service providing part may provide the mobility user with information of overviews of facilities, operating hours, routes, fees, etc., about sports facilities, hot springs, amusement parks, tourist attractions, art museums, etc., for example.

According to this embodiment, based on the VLF data VD obtained from the mobility user riding in the mobility 11, a change in the health state of the mobility user can be estimated by relatively comparing the current VLF data VDA during current riding with the past VLF data VDB during past riding. Based on the change in the health state estimated in this manner, a service related to the health state can be provided to the mobility user.

### Embodiment 1.2

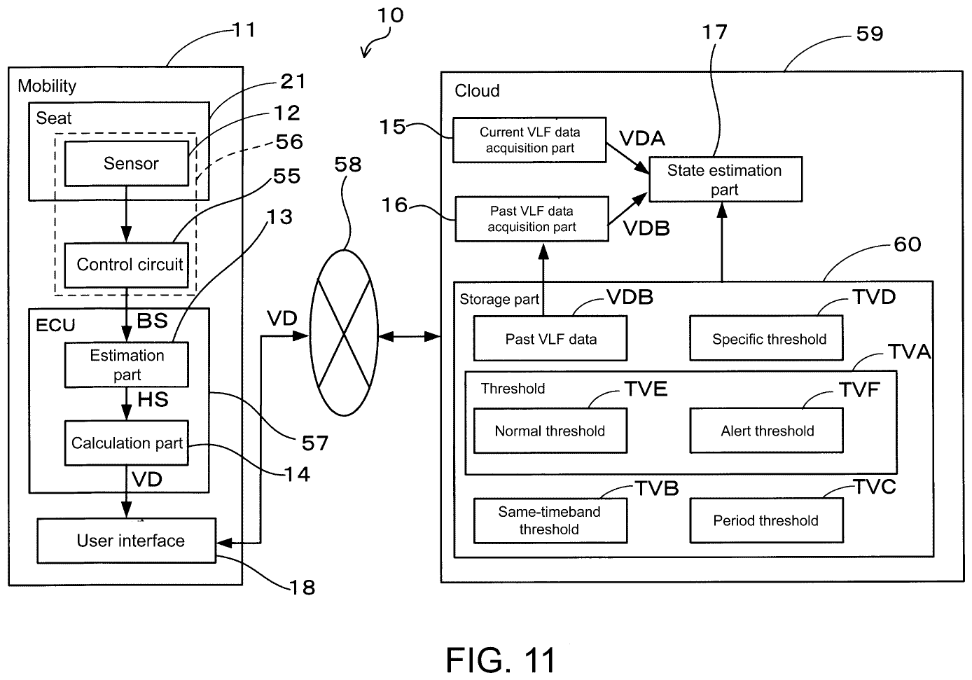

Next, referring to FIG. 11 to FIG. 13, Embodiment 1.2 will be described. Among reference signs used from Embodiment 1.2 onward, reference signs that are identical to those used in previous embodiments represent constituent elements and the like similar to those in the previous embodiments unless otherwise specified.

As shown in FIG. 11, the health information service providing device 10 of this embodiment differs from Embodiment 1.1 in that the storage part 60 stores a specific threshold TVD, and the threshold TVA includes a normal threshold TVE and an alert threshold TVF. The alert threshold TVF is a value smaller than the normal threshold TVE.

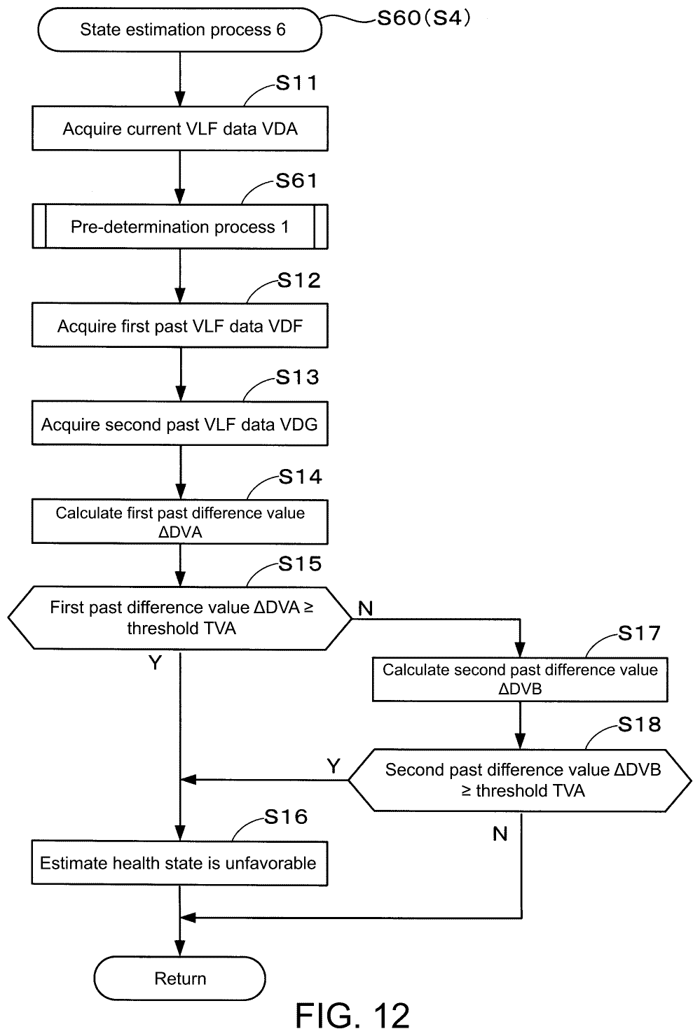

In addition, this embodiment differs from Embodiment 1.1 in that a state estimation process 6 (S60) is executed instead of the state estimation process 1 (S10) (refer to FIG. 12).

FIG. 12 shows a flowchart of the state estimation process 6 (S60). As will be described in detail later, the state estimation process 6 (S60) of this embodiment is an embodiment that uses the normal threshold TVE when current VLF data VDA is greater than the specific threshold TVD, and uses the alert threshold TVF when the current VLF data VDA is equal to or less than the specific threshold TVD. Upon execution of the state estimation process 6 (S60), the current VLF data acquisition part 15 acquires current VLF data VDA (S11).

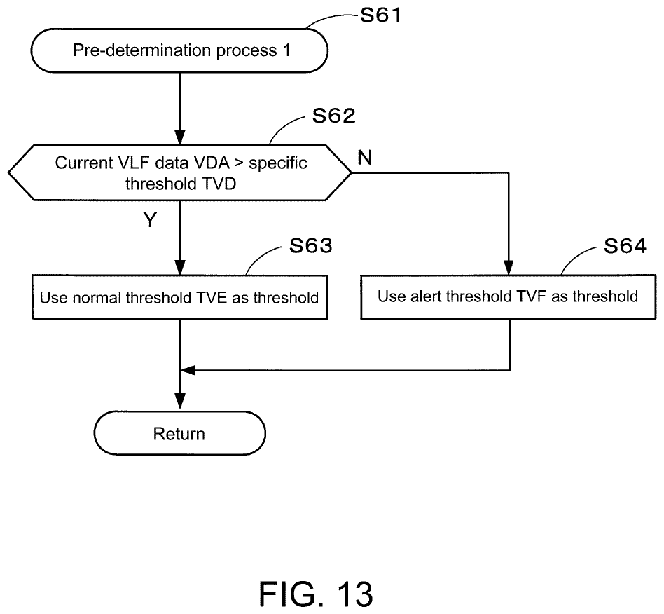

Next, the state estimation part 17 executes a pre-determination process 1 (S61). FIG. 13 shows a flowchart of a subroutine of the pre-determination process 1 (S61).

Upon execution of the pre-determination process 1 (S61), the state estimation part 17 determines whether the current VLF data VDA exceeds the specific threshold TVD stored in the storage part 60 (S62). In other words, this embodiment includes a process that focuses on the value of the current VLF data VDA itself.

In the case where the current VLF data VDA exceeds the specific threshold TVD (S62: Y), the state estimation part 17 uses the normal threshold TVE as the threshold TVA (S63). Accordingly, the pre-determination process 1 (S61) is ended, and the flow returns to the state estimation process 6 (S60).

In contrast, in the case where the current VLF data VDA is equal to or less than the specific threshold TVD (S62: N), the state estimation part 17 uses the alert threshold TVF as the threshold TVA (S64). Accordingly, the pre-determination process 1 (S61) is ended, and the flow returns to the state estimation process 6 (S60).

Returning to the state estimation process 6 (S60), since subsequent processes (S12 to S18) are the same as those in the state estimation process 1 (S10) of Embodiment 1.1, repeated descriptions thereof will be omitted.

Next, effects of this embodiment will be described. As described above, the alert threshold TVF is a value smaller than the normal threshold TVE. Thus, upon using the alert threshold TVF as the threshold TVA, it becomes more likely to determine that the health state of the mobility user has changed in an unfavorable direction.

The current VLF data VDA is information related to a current health state of the mobility user. As described above, if the value of the current VLF data VDA is large, the health state of the mobility user is estimated to be favorable, and if the value of the current VLF data VDA is small, the health state of the mobility user is estimated to be unfavorable. In the case where the current VLF data VDA is large enough to exceed the specific threshold TVD, since it is estimated that the health state of the mobility user is in a favorable tendency, the normal threshold TVE is used as the threshold TVA.

In contrast, in the case where the current VLF data VDA is equal to or less than the specific threshold TVD, it is estimated that the health state of the mobility user is already in an unfavorable state. Thus, in the case where it is estimated that the health state of the mobility user is in an unfavorable state by using the alert threshold TVF, which is a value smaller than the normal threshold TVE, as the threshold TVA, it becomes even more likely to estimate that the health state of the mobility user is unfavorable. Accordingly, it is possible to prevent the mobility user from forcing themselves to be active despite being in an unfavorable health state.

### Embodiment 1.3

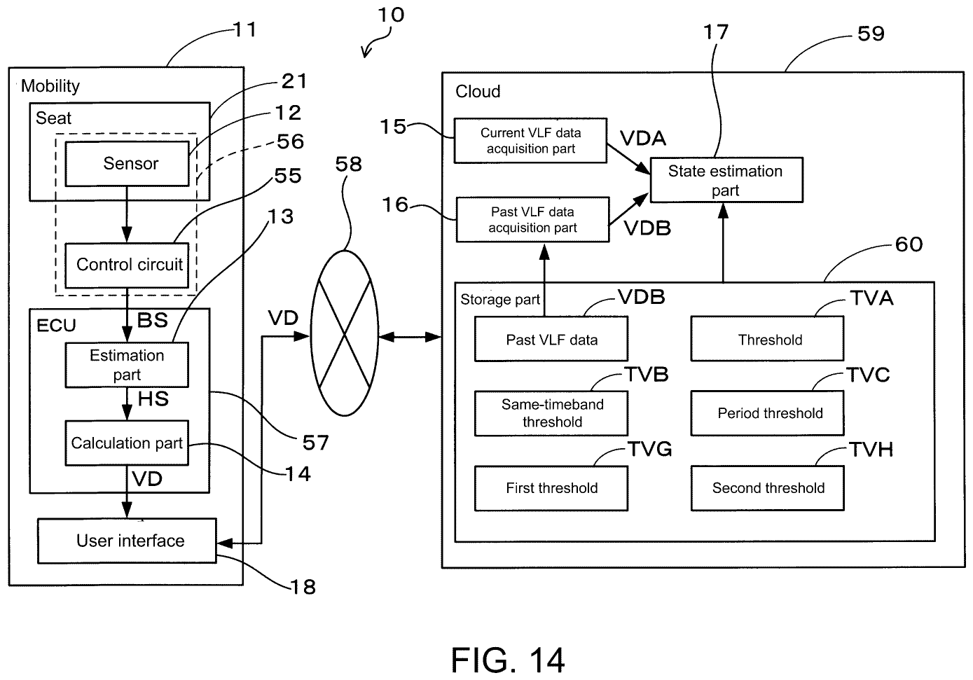

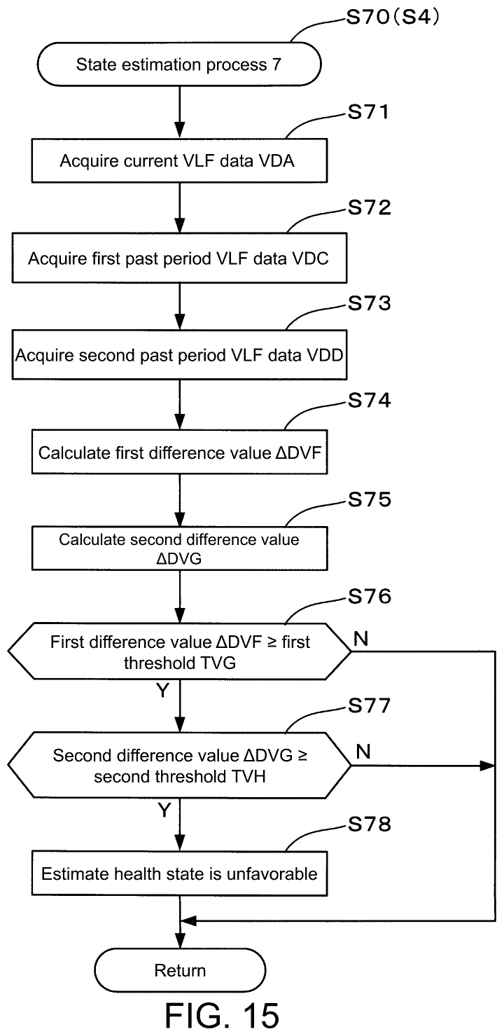

Next, referring to FIG. 14 to FIG. 15, Embodiment 1.3 will be described. As shown in FIG. 14, the health information service providing device 10 of this embodiment differs from Embodiment 1.1 in that the storage part 60 stores a first threshold TVG and a second threshold TVH.

In addition, this embodiment differs from Embodiment 1.1 in that a state estimation process 7 (S70) is executed instead of the state estimation process 1 (S10) (refer to FIG. 15).

FIG. 15 shows a flowchart of the state estimation process 7 (S70). As will be described in detail later, the state estimation process 7 (S70) of this embodiment is an embodiment that estimates that the health state of the mobility user has changed in an unfavorable direction when a first difference value ΔDVF obtained by subtracting current VLF data VDA from first past period VLF data VDC is equal to or greater than the first threshold TVG, and a second difference value ΔDVG obtained by subtracting the current VLF data VDA from second past period VLF data VDD is equal to or greater than the second threshold TVH. Upon execution of the state estimation process 7 (S70), the current VLF data acquisition part 15 acquires current VLF data VDA, which is VLF data VD during a current riding or a most recent riding (S71).

Next, the past VLF data acquisition part 16 acquires first past period VLF data VDC, which is a statistical representative value of VLF data VD in a first period prior to the current riding or the most recent riding (S72).

Next, the past VLF data acquisition part 16 acquires second past period VLF data VDD, which is a statistical representative value of VLF data VD in a second period that is longer than the first period of the first past period VLF data VDC (S73).

Next, the state estimation part 17 calculates a first difference value ΔDVF obtained by subtracting the current VLF data VDA from the first past period VLF data VDC (S74).

Next, the state estimation part 17 calculates a second difference value ΔDVG obtained by subtracting the current VLF data VDA from the second past period VLF data VDD (S75).

Next, the state estimation part 17 determines whether the first difference value ΔDVF is equal to or greater than the first threshold TVG (S76), and further determines whether the second difference value ΔDVG is equal to or greater than the second threshold TVH (S77).

When the first difference value ΔDVF is equal to or greater than the first threshold TVG (S76; Y), and the second difference value ΔDVG is equal to or greater than the second threshold TVH (S77: Y), the state estimation part 17 estimates that the health state of the mobility user has changed in an unfavorable direction (S78). Accordingly, the state estimation process 7 (S70) is ended.

In contrast, when the first difference value ΔDVF is smaller than the first threshold TVG (S76: N), and when the second difference value ΔDVG is smaller than the second threshold TVH (S77: N), the state estimation part 17 ends the state estimation process 7 (S70).

According to this embodiment, the first past period VLF data VDC in the first period and the second past period VLF data VDD in the second period, which is longer than the first period, are used as comparison targets compared with the current VLF data VDA. By comparing in different periods in this manner, it is possible to estimate the health state of the mobility user in a more multifaceted manner. However, the combination of the first period and the second period is not particularly limited, and any combination may be appropriately selected, such as a combination of a previous-time riding and a previous-day riding, a combination of the previous-time riding and most recent one week, or a combination of the previous-day riding and most recent one week, for example.

### Embodiment 1.4

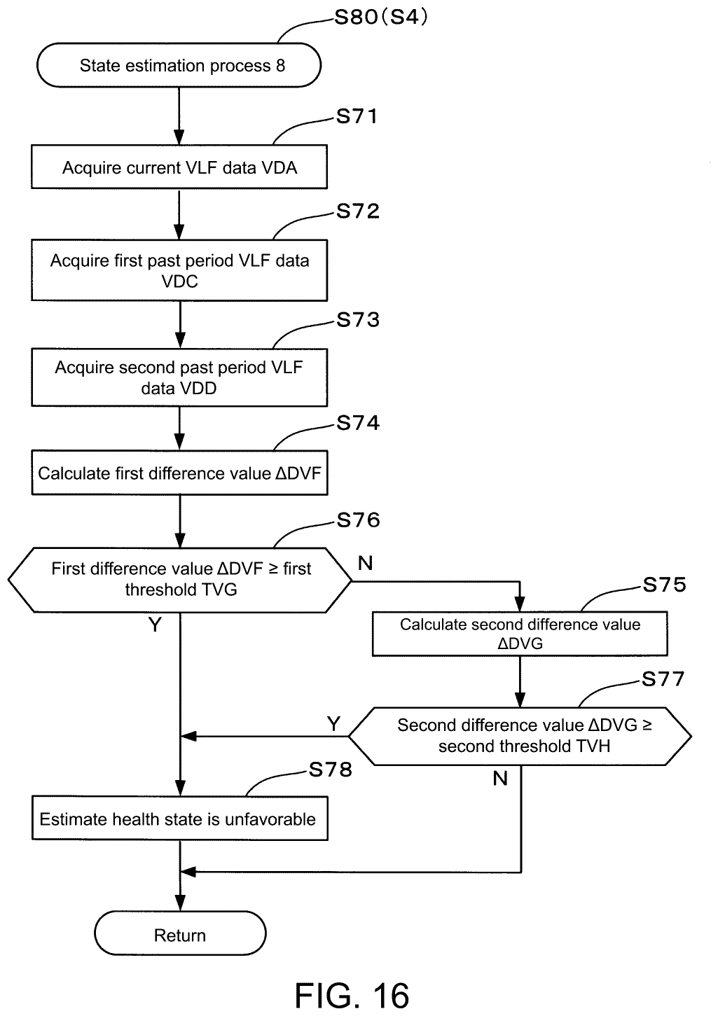

Next, referring to FIG. 16, Embodiment 1.4 will be described. This embodiment differs from Embodiment 1.3 in that a state estimation process 8 (S80) is executed instead of the state estimation process 7 (S70) (refer to FIG. 16).

As shown in FIG. 16, the state estimation process 8 is identical to the state estimation process 7 (S70) described in FIG. 15 from S71 to S74, and processes from S75 onward are different. Repeated descriptions of processes that are the same as those in the state estimation process 7 (S70) will be omitted.

In this embodiment, the state estimation process 8 (S80) estimates that the health state of the mobility user has changed in an unfavorable direction in the case where a first difference value ΔDVF obtained by subtracting current VLF data VDA from first past period VLF data VDC is equal to or greater than the first threshold TVG. In addition, it is estimated that the health state of the mobility user has changed in an unfavorable direction in the case where the first difference value ΔDVF is smaller than the first threshold TVG, and a second difference value ΔDVG obtained by subtracting the current VLF data VDA from second past period VLF data VDD is equal to or greater than the second threshold TVH.

As shown in FIG. 16, after calculating a first difference value ΔDVF (S74), the state estimation part 17 determines whether the first difference value ΔDVF is equal to or greater than the first threshold TVG (S76).

When the first difference value ΔDVF is equal to or greater than the first threshold TVG (S76: Y), the state estimation part 17 estimates that the health state of the mobility user has changed in an unfavorable direction (S78). Accordingly, the state estimation process 8 (S80) is ended.

In contrast, when the first difference value ΔDVF is smaller than the first threshold TVG (S76: N), the state estimation part 17 calculates a second difference value ΔDVG (S75).

Next, the state estimation part 17 determines whether the second difference value ΔDVG is equal to or greater than the second threshold TVH (S77).

When the second difference value ΔDVG is equal to or greater than the second threshold TVH (S77: Y), the state estimation part 17 estimates that the health state of the mobility user has changed in an unfavorable direction (S78). Accordingly, the state estimation process 8 (S80) is ended.

In contrast, when the second difference value ΔDVG is smaller than the second threshold TVH (S77: N), the state estimation process 8 (S80) is ended.

According to this embodiment, the first past period VLF data VDC in the first period and the second past period VLF data VDD in the second period, which is longer than the first period, are used as comparison targets compared with the current VLF data VDA. Accordingly, by comparing in different periods, it is possible to estimate the health state of the mobility user in a more multifaceted manner.

### Embodiment 1.5

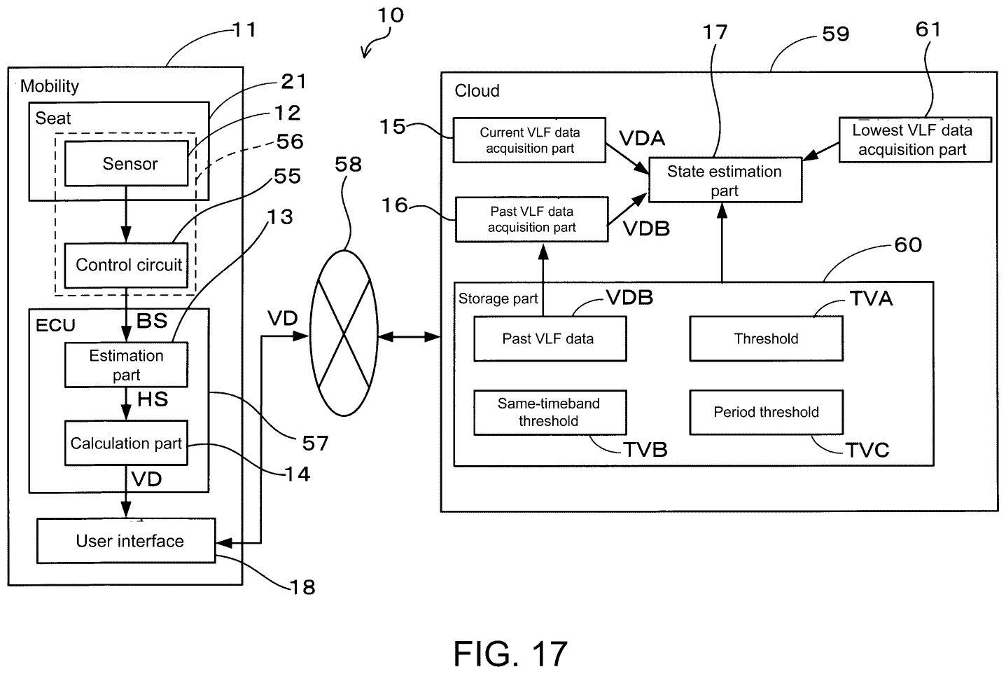

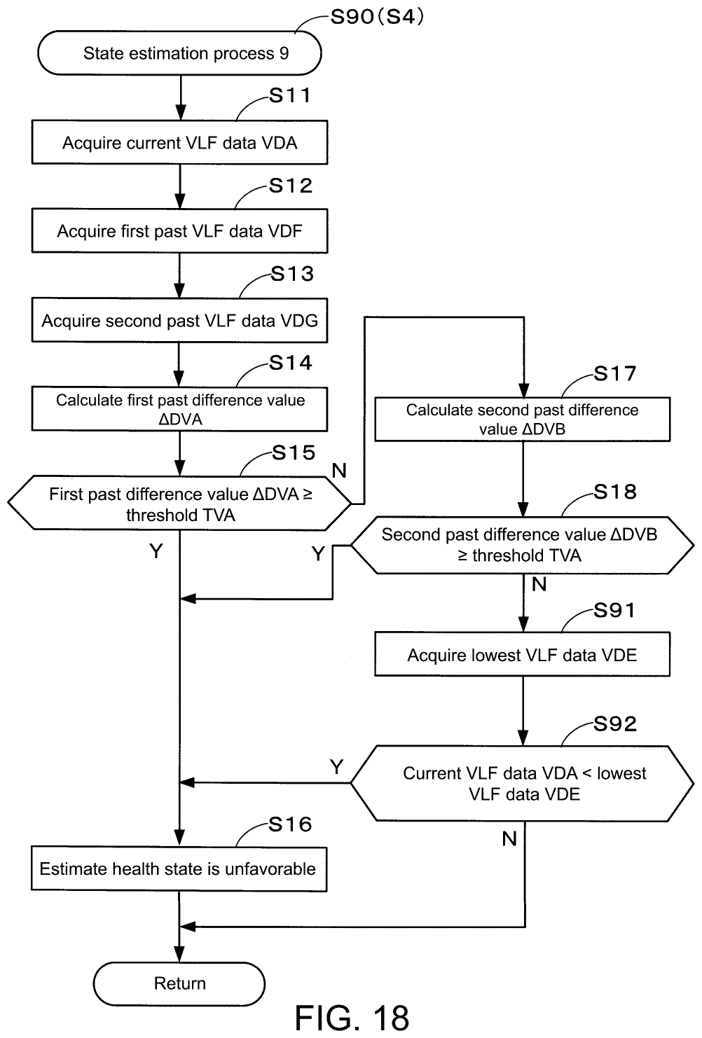

Next, referring to FIG. 17 to FIG. 18, Embodiment 1.5 will be described. As shown in FIG. 17, the health information service providing device 10 of this embodiment differs from Embodiment 1.1 in that the cloud 59 includes a lowest VLF data acquisition part 61. Lowest VLF data VDE is a lowest value of VLF data VD over a period of time longer than a specific period in the past in past VLF data VDB.

In addition, this embodiment differs from Embodiment 1.1 in that a state estimation process 9 (S90) is executed instead of the state estimation process 1 (S10) (refer to FIG. 18).

FIG. 18 shows a flowchart of the state estimation process 9 (S90). As will be described in detail later, the state estimation process 9 (S90) of this embodiment is an embodiment that compares current VLF data VDA with past VLF data VDB, and then compares the current VLF data VDA with lowest VLF data VDE, which is lowest VLF data VD measured in the past. The state estimation process 9 (S90) differs from the state estimation process 1 (S10) in FIG. 6 in executing a process (S91) in which the lowest VLF data acquisition part 61 acquires the lowest VLF data VDE, and a process (S92) in which the state estimation part 17 determines whether the current VLF data VDA is smaller than the lowest VLF data VDE. Thus, in the following description, processes that are the same as those in the state estimation process 1 (S10) will be labeled with the same reference signs, and repeated descriptions thereof will be omitted.

As shown in FIG. 18, in the state estimation process 9 (S90), when the second past difference value ΔDVB is smaller than the threshold TVA (S18: N), the lowest VLF data acquisition part 61 acquires lowest VLF data VDE from the storage part 60 (S91).

Next, the state estimation part 17 determines whether the current VLF data VDA is smaller than the lowest VLF data VDE (S92). In other words, in this embodiment, the value of the current VLF data VDA is compared with a lowest value of VLF data VD acquired in the past.

When the current VLF data VDA is smaller than the lowest VLF data VDE (S92: Y), the state estimation part 17 estimates that the health state of the mobility user has changed in an unfavorable direction (S16). Accordingly, the state estimation process 9 (S90) is ended.

In contrast, when the current VLF data VDA is equal to or greater than the lowest VLF data VDE (S92: N), the state estimation process 9 (S90) is ended.

In a simple relative comparison of the current VLF data VDA with the first past VLF data VDF and the second past VLF data VDG, in the case where the first past difference value ΔDVA is equal to or greater than the threshold TVA, and the second past difference value ΔDVB is equal to or greater than the threshold TVA, the state estimation part 17 does not determine that the health state of the mobility user has changed in an unfavorable direction. However, according to this embodiment, even if the first past difference value ΔDVA is equal to or greater than the threshold TVA and the second past difference value ΔDVB is equal to or greater than the threshold TVA, in the case where the current VLF data VDA is smaller than the lowest VLF data VDE, the state estimation part 17 estimates that the health state of the mobility user has changed in an unfavorable direction. Accordingly, it is possible to estimate the health state of the mobility user more accurately.

### Embodiment 1.6

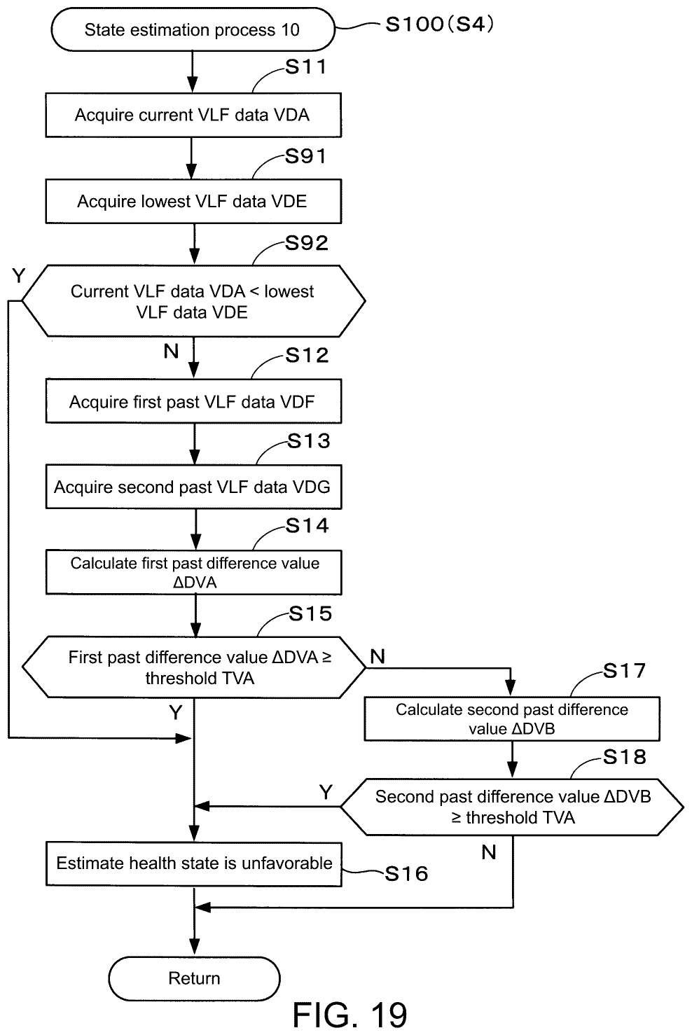

Next, referring to FIG. 19, Embodiment 1.6 will be described. This embodiment differs from Embodiment 1.5 in that a state estimation process 10 (S100) is executed instead of the state estimation process 9 (S90).

FIG. 19 shows a flowchart of the state estimation process 10 (S100). As will be described in detail later, this embodiment compares current VLF data VDA with lowest VLF data VDE, which is lowest VLF data VD measured in the past, before comparing the current VLF data VDA with past VLF data VDB. The state estimation process 10 (S100) differs from the state estimation process 9 (S90) in FIG. 18 in executing a process (S91) in which the lowest VLF data acquisition part 61 acquires the lowest VLF data VDE and a process (S92) in which the state estimation part 17 determines whether the current VLF data VDA is smaller than the lowest VLF data VDE, after the current VLF data acquisition part 15 acquires the current VLF data VDA (S11).

As shown in FIG. 19, after the current VLF data acquisition part 15 acquires current VLF data VDA (S11), the lowest VLF data acquisition part 61 acquires lowest VLF data VDE from the storage part 60 (S91).

Next, the state estimation part 17 determines whether the current VLF data VDA is smaller than the lowest VLF data VDE (S92).

When the current VLF data VDA is smaller than the lowest VLF data VDE (S92: Y), the state estimation part 17 estimates that the health state of the mobility user has changed in an unfavorable direction (S16). Accordingly, the state estimation process 10 (S100) is ended.

In contrast, when the current VLF data VDA is equal to or greater than the lowest VLF data VDE (S92: N), processes from S12 onward are executed. Since the processes of S12 to S18 are the same as those in the state estimation process 1 (S10) in FIG. 6, repeated descriptions thereof will be omitted.

According to this embodiment, in the case where the current VLF data VDA is smaller than the lowest VLF data VDE, the state estimation part 17 immediately estimates that the health state of the mobility user has changed in an unfavorable direction. Accordingly, it is possible to promptly estimate that the health state of the mobility user has changed in an unfavorable direction.

### Embodiment 1.7

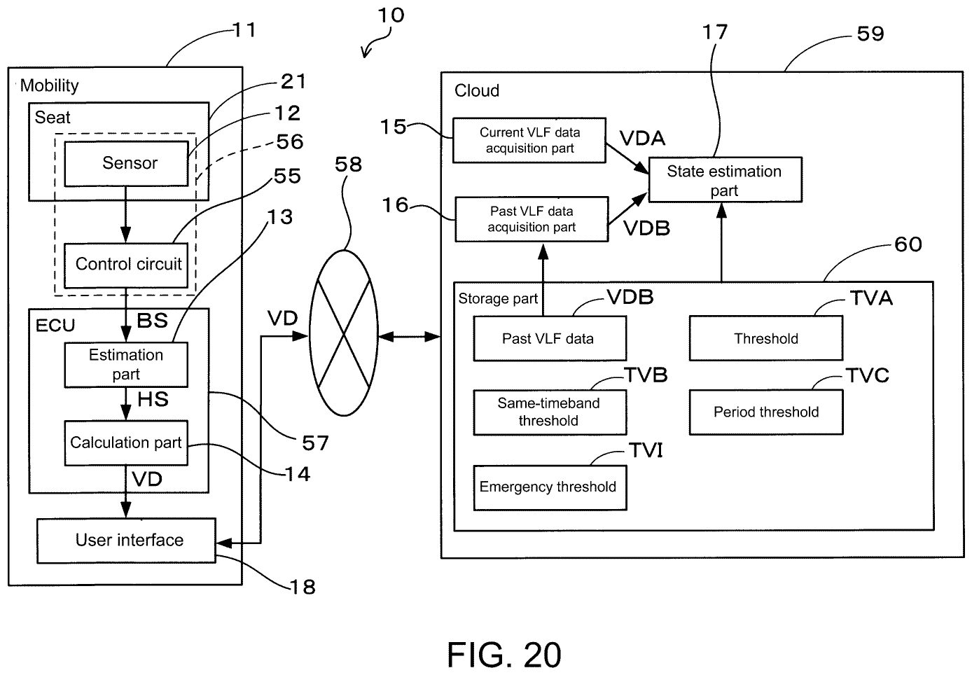

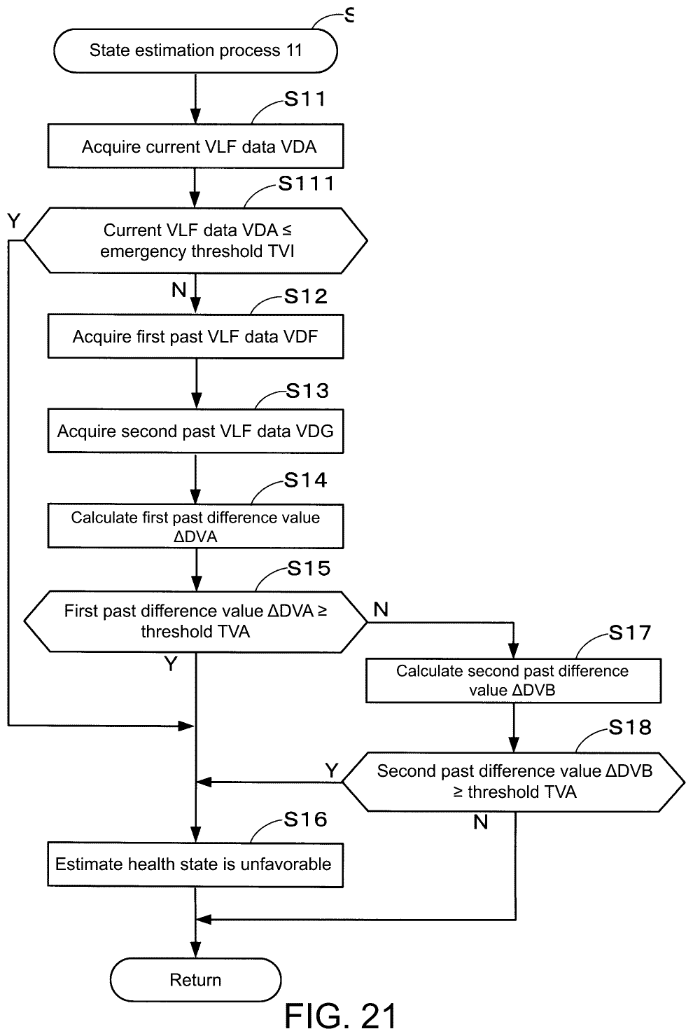

Next, referring to FIG. 20 to FIG. 21, Embodiment 1.7 will be described. As shown in FIG. 20, this embodiment differs from Embodiment 1.1 in that the storage part 60 stores an emergency threshold TVI.

In addition, this embodiment differs from Embodiment 1.1 in that a state estimation process 11 (S110) is executed instead of the state estimation process 1 (S10).

FIG. 21 shows a flowchart of the state estimation process 11 (S110). As will be described in detail later, the state estimation process 11 (S110) of this embodiment is an embodiment that compares current VLF data VDA with the emergency threshold TVI before comparing the current VLF data VDA with past VLF data VDB. Upon execution of the state estimation process 11 (S110), the current VLF data acquisition part 15 acquires current VLF data VDA (S11).

Next, the state estimation part 17 determines whether the current VLF data VDA is equal to or less than the emergency threshold TVI (S111). When the current VLF data VDA is equal to or less than the emergency threshold TVI (S111: Y), the state estimation part 17 estimates that the health state of the mobility user has changed in an unfavorable direction (S16). Accordingly, the state estimation process 11 (S110) is ended.

In contrast, when the current VLF data VDA is greater than the emergency threshold TVI (S111: N), processes of S12 to S18 are executed. Since the processes of S12 to S18 are the same as those in the state estimation process 1 (S10) of Embodiment 1.1, repeated descriptions thereof will be omitted.

According to this embodiment, in the case where the current VLF data VDA is equal to or less than the emergency threshold TVI, the state estimation part 17 immediately estimates that the health state of the mobility user has changed in an unfavorable direction. Accordingly, it is possible to promptly estimate that the health state of the mobility user has changed in an unfavorable direction.

However, the embodiment may also compare the current VLF data VDA with the emergency threshold TVI after comparing the current VLF data VDA with the past VLF data VDB.

### Embodiment 1.8

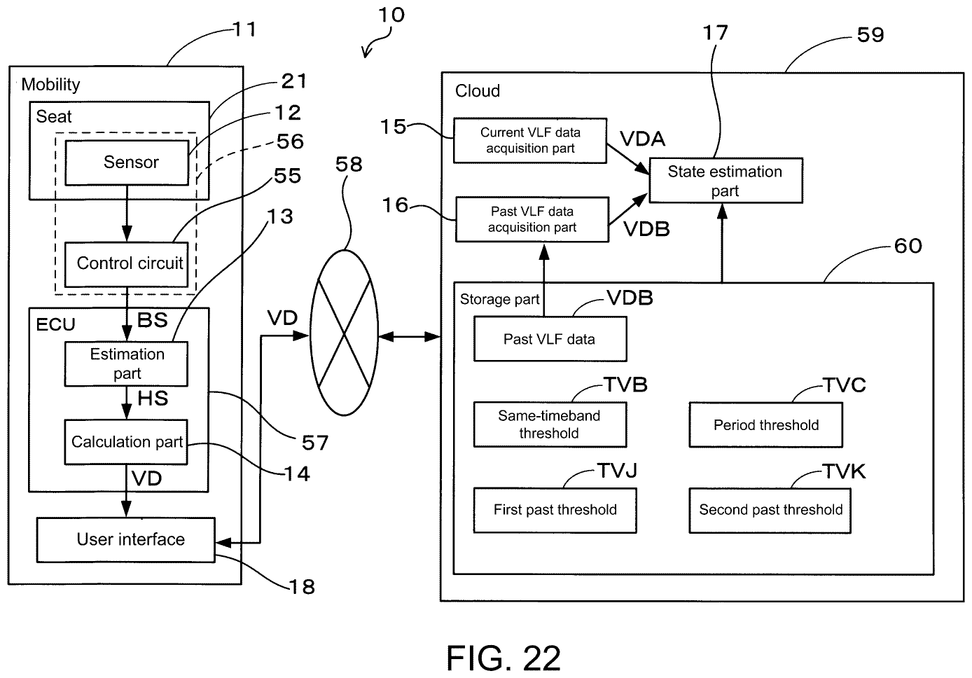

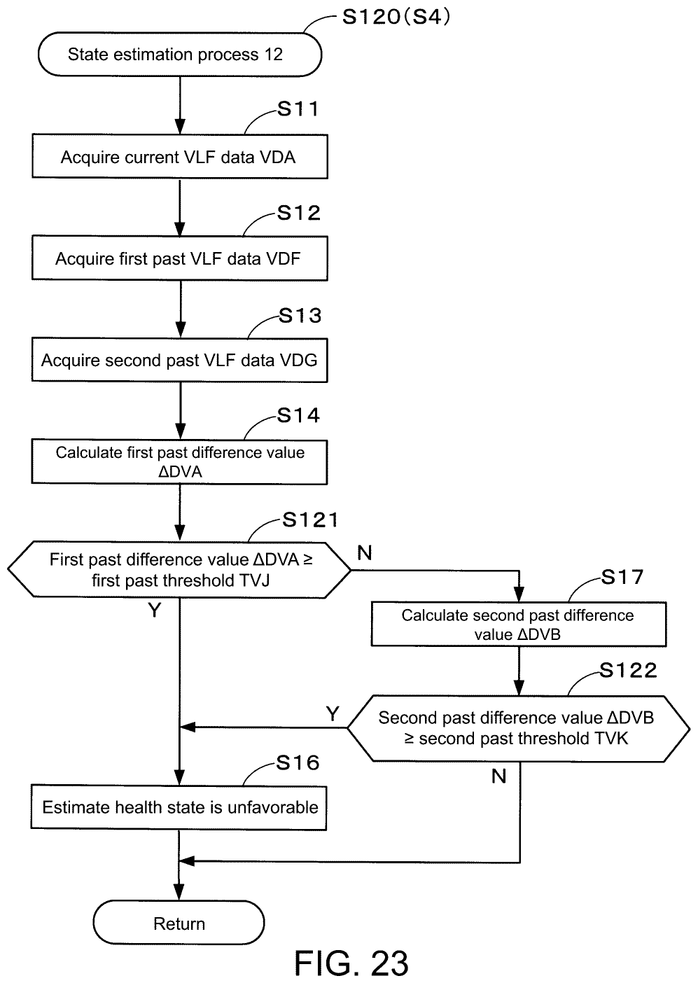

Next, referring to FIG. 22 to FIG. 23, Embodiment 1.8 will be described. As shown in FIG. 22, the storage part 60 according to this embodiment differs from Embodiment 1.1 in including a first past threshold TVJ (an example of a first comparison criterion) and a second past threshold TVK (an example of a second comparison criterion) instead of the threshold TVA. This embodiment estimates the health state of the mobility user using the first past threshold TVJ and the second past threshold TVK.

In addition, this embodiment differs from Embodiment 1.1 in that a state estimation process 12 (S120) is executed instead of the state estimation process 1 (S10) (refer to FIG. 23).

As shown in FIG. 23, the state estimation process 12 (S120) differs from the state estimation process 1 (S10) in that, compared to FIG. 6, S121 is executed instead of S15, and S122 is executed instead of S18. The same processes as those in the state estimation process 1 (S10) will be labeled with the same reference signs, and repeated descriptions thereof will be omitted.

As will be described in detail later, the state estimation process 12 (S120) of this embodiment is an embodiment that determines on a first past difference value ΔDVA using the first past threshold TVJ, and determines on a second past difference value ΔDVB using the second past threshold TVK.

After calculating a first past difference value ΔDVA (S14), the state estimation part 17 determines whether the first past difference value ΔDVA is equal to or greater than the first past threshold TVJ (S121).

When the first past difference value ΔDVA is equal to or greater than the first past threshold TVJ (S121: Y), the state estimation part 17 estimates that the health state of the mobility user has changed in an unfavorable direction (S16). Accordingly, the state estimation process 12 (S120) is ended.

In contrast, when the first past difference value ΔDVA is smaller than the first past threshold TVJ (S121: N), the state estimation part 17 calculates a second past difference value ΔDVB (S17). Next, the state estimation part 17 determines whether the second past difference value ΔDVB is equal to or greater than the second past threshold TVK (S122).

When the second past difference value ΔDVB is equal to or greater than the second past threshold TVK (S122: Y), the state estimation part 17 estimates that the health state of the mobility user has changed in an unfavorable direction (S16). Accordingly, the state estimation process 12 (S120) is ended.

In contrast, when the second past difference value ΔDVB is smaller than the second past threshold TVK (S122: N), the state estimation process 12 (S120) is ended.

According to this embodiment, determination is performed on the first past difference value ΔDVA using the first past threshold TVJ, and determination is performed on the second past difference value ΔDVB using the second past threshold TVK. Accordingly, the health state of the mobility user can be determined precisely.

### Embodiment 1.9

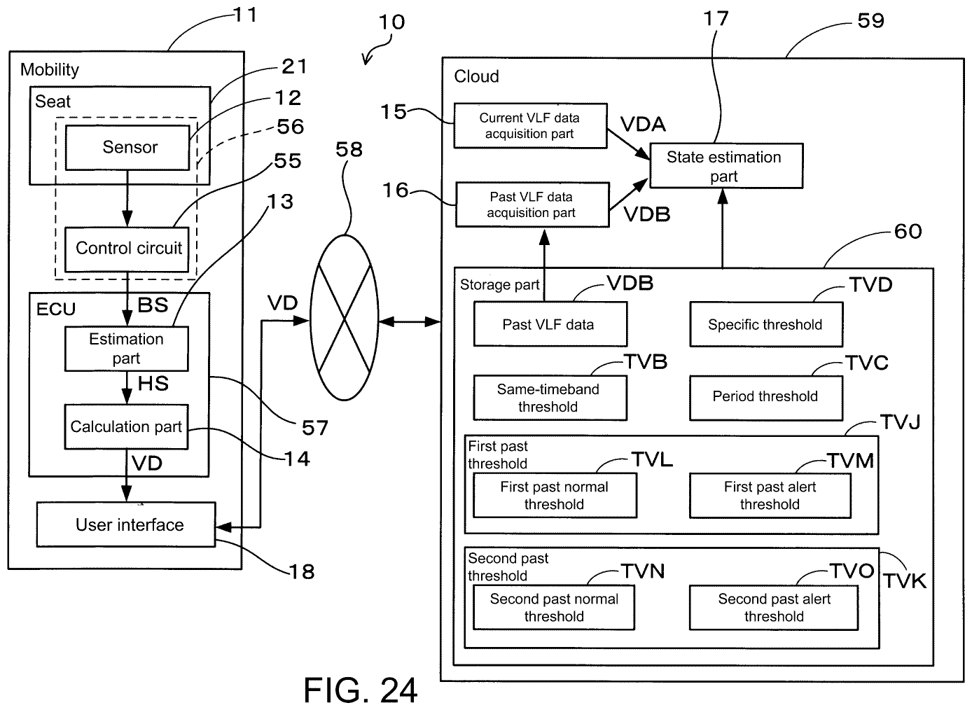

Next, referring to FIG. 24 to FIG. 26, Embodiment 1.9 will be described. As shown in FIG. 24, the storage part 60 of this embodiment differs from Embodiment 1.8 in that the storage part 60 stores a specific threshold TVD, the first past threshold TVJ includes a first past normal threshold TVL and a first past alert threshold TVM, and the second past threshold TVK includes a second past normal threshold TVN and a second past alert threshold TVO. The first past alert threshold TVM is a value smaller than the first past normal threshold TVL. The second past alert threshold TVO is a value smaller than the second past normal threshold TVN.

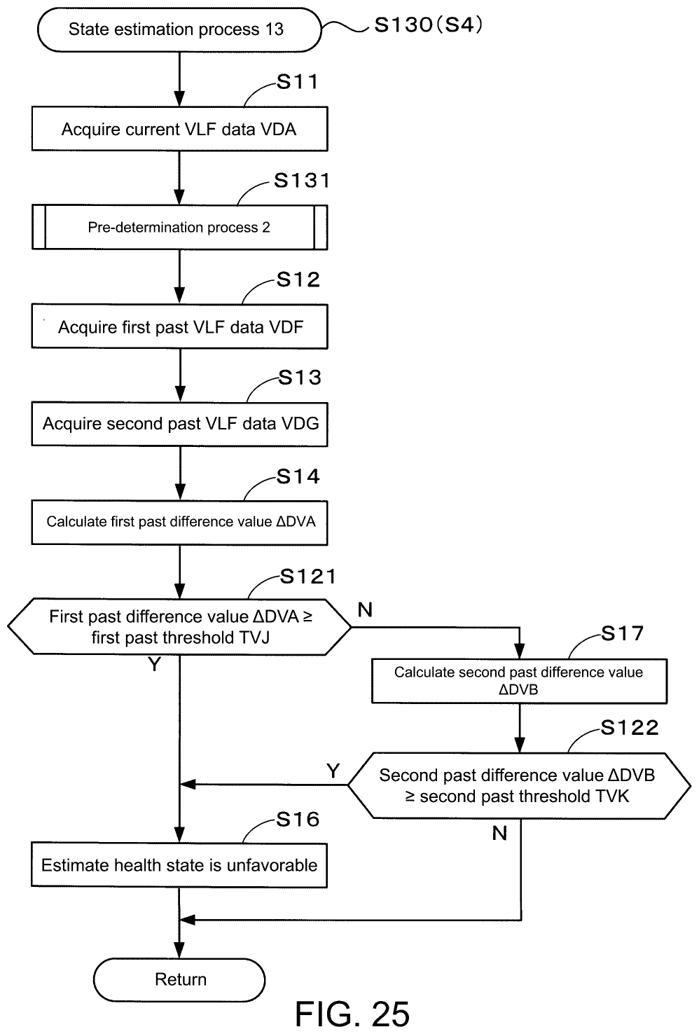

In addition, this embodiment differs from Embodiment 1.8 in that a state estimation process 13 (S130) is executed instead of the state estimation process 12 (S120) (FIG. 25).

As will be described in detail later, the state estimation process 13 (S130) of this embodiment is an embodiment that estimates the health state of the mobility user using the first past normal threshold TVL and the second past normal threshold TVN in the case where current VLF data VDA exceeds the specific threshold TVD, and estimates the health state of the mobility user using the first past alert threshold TVM and the second past alert threshold TVO in the case where the current VLF data VDA is equal to or less than the specific threshold TVD.

As shown in FIG. 25, the state estimation process 13 (S130) differs from the state estimation process 12 (S120) in FIG. 23 in that a pre-determination process 2 (S131) is executed after S11 is executed. The same processes as those in the state estimation process 12 (S120) will be labeled with the same reference signs, and repeated descriptions thereof will be omitted.

As shown in FIG. 25, upon execution of the state estimation process 13 (S130), the current VLF data acquisition part 15 acquires current VLF data VDA (S11). Next, the state estimation part 17 executes the pre-determination process 2 (S131).

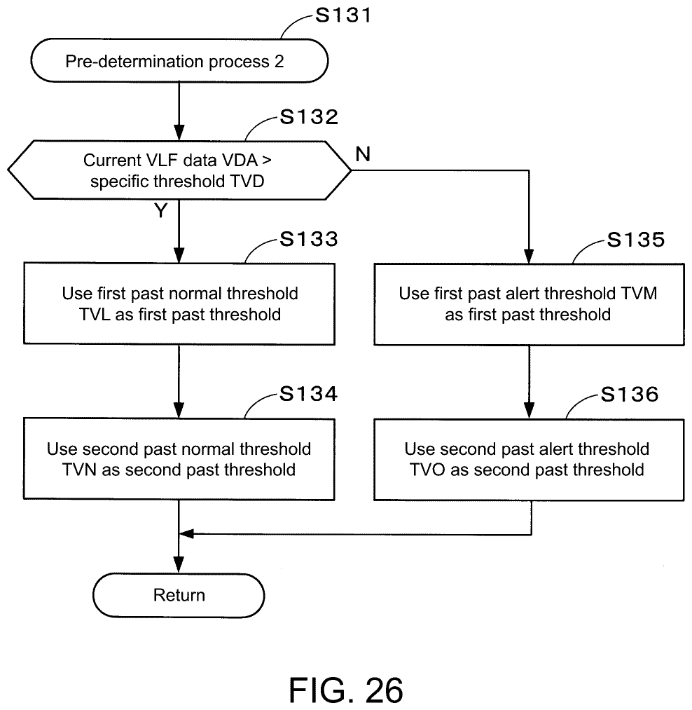

FIG. 26 shows a flowchart of a subroutine of the pre-determination process 2 (S131). Upon execution of the pre-determination process 2 (S131), the state estimation part 17 determines whether the current VLF data VDA exceeds the specific threshold TVD stored in the storage part 60 (S132).

When the current VLF data VDA exceeds the specific threshold TVD (S132: Y), the state estimation part 17 uses the first past normal threshold TVL as the first past threshold TVJ (S133), and uses the second past normal threshold TVN as the second past threshold TVK (S134). Accordingly, the pre-determination process 2 (S131) is ended.

In contrast, when the current VLF data VDA is equal to or less than the specific threshold TVD (S132: N), the state estimation part 17 uses the first past alert threshold TVM as the first past threshold TVJ (S135), and uses the second past alert threshold TVO as the second past threshold TVK (S136). Accordingly, the pre-determination process 2 (S131) is ended.

Returning to FIG. 25, since processes of S12 to S14, S121, S16, S17, and S122 are the same as those in the state estimation process 12 (S120) in FIG. 23, the same reference signs will be labeled and repeated descriptions thereof will be omitted.

Next, effects of this embodiment will be described. As described above, the first past alert threshold TVM is a value smaller than the first past normal threshold TVL, and the second past alert threshold TVO is a value smaller than the second past normal threshold TVN. Thus, upon using the first past alert threshold TVM as the first past threshold TVJ and using the second past alert threshold TVO as the second past threshold TVK, it becomes more likely to determine that the health state of the mobility user has changed in an unfavorable direction.

In addition, in the case where the current VLF data VDA is equal to or less than the specific threshold TVD, it is estimated that the health state of the mobility user is already in an unfavorable state. Thus, when the current VLF data VDA is equal to or less than the specific threshold TVD, in the case where it is estimated that the health state of the mobility user is in an unfavorable state by using, as the threshold TVA, the first past alert threshold TVM as the first past threshold TVJ and using the second past alert threshold TVO as the second past threshold TVK, it becomes even more likely to estimate that the health state of the mobility user is unfavorable. Accordingly, it is possible to prevent the mobility user from forcing themselves to be active despite being in an unfavorable health state.

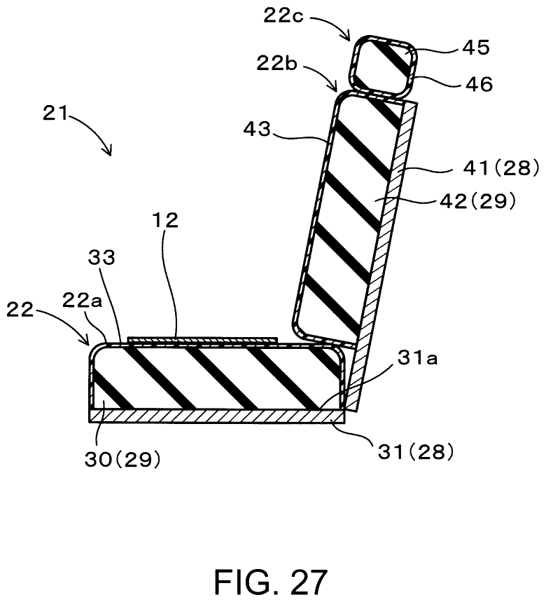

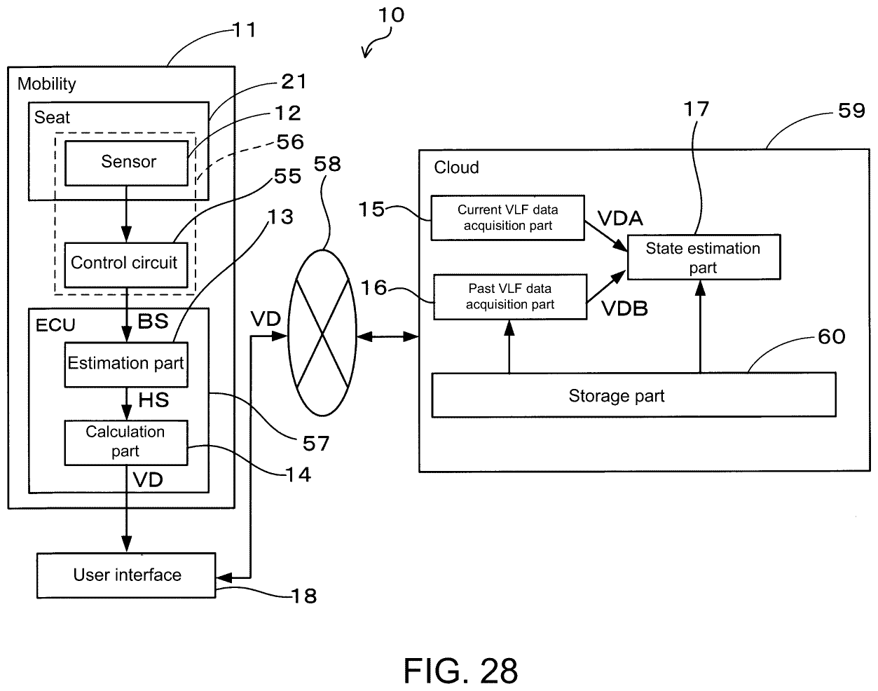

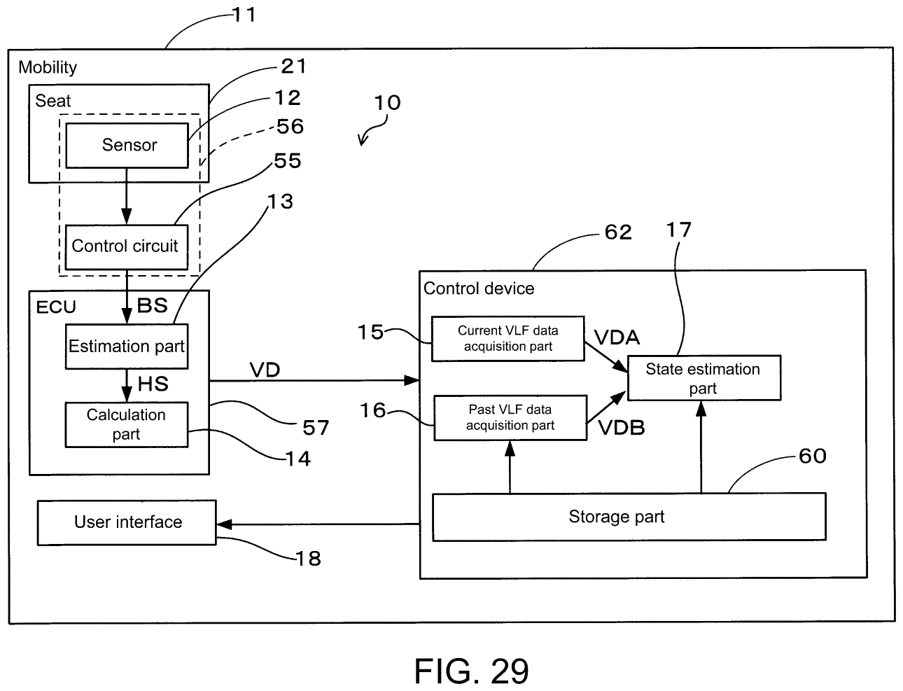

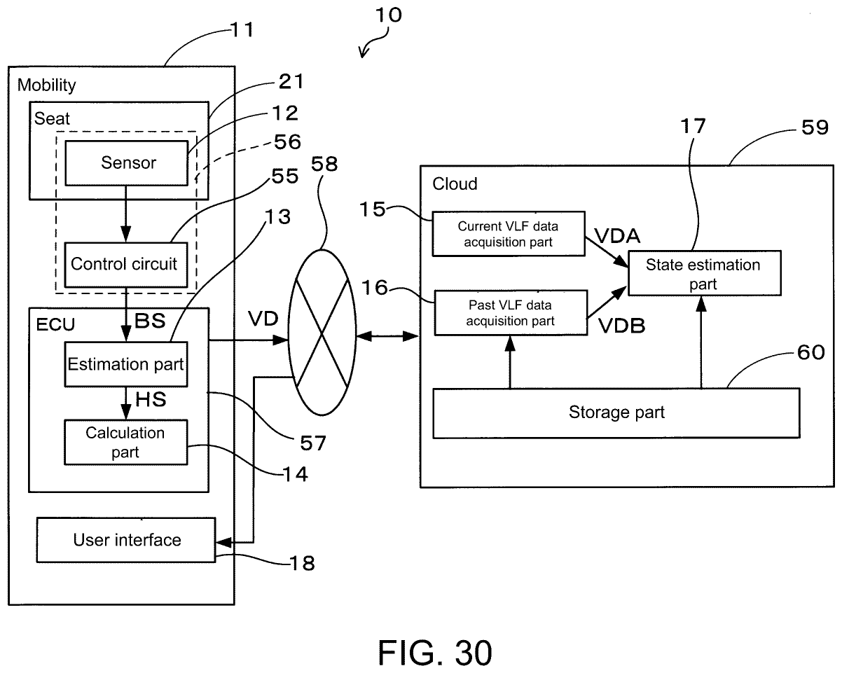

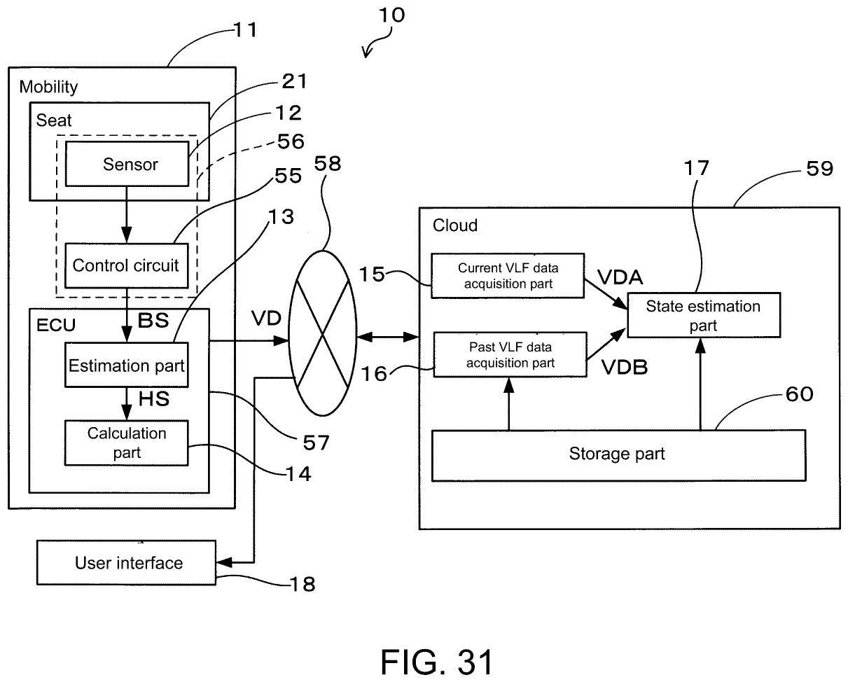

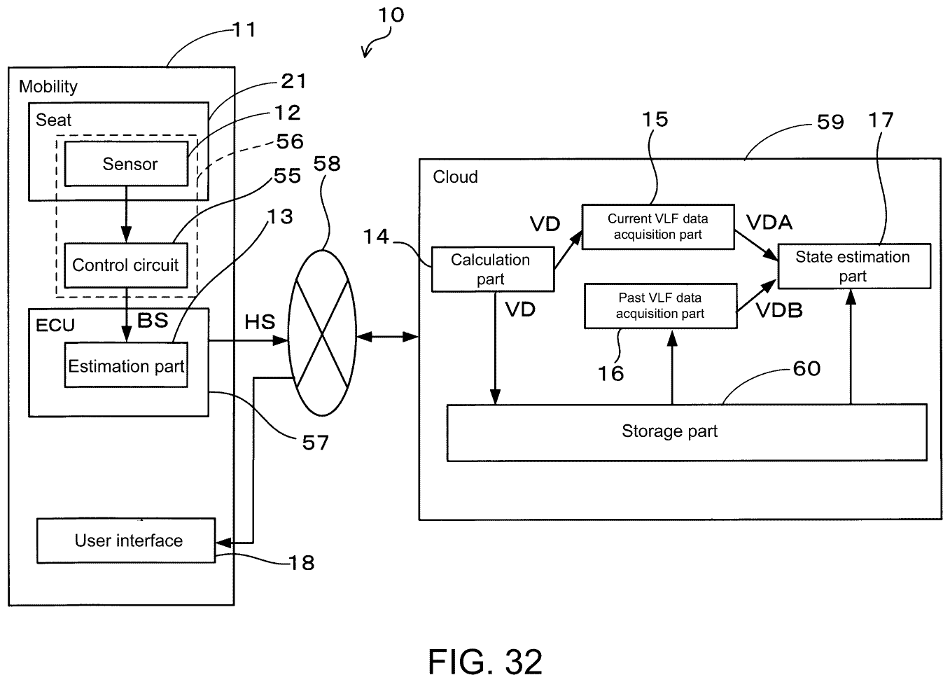

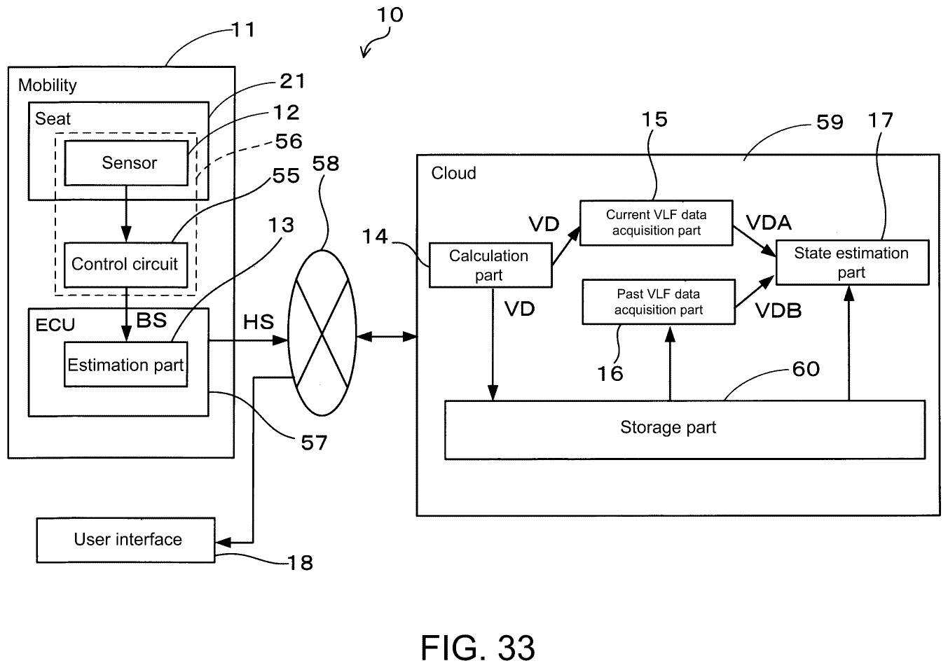

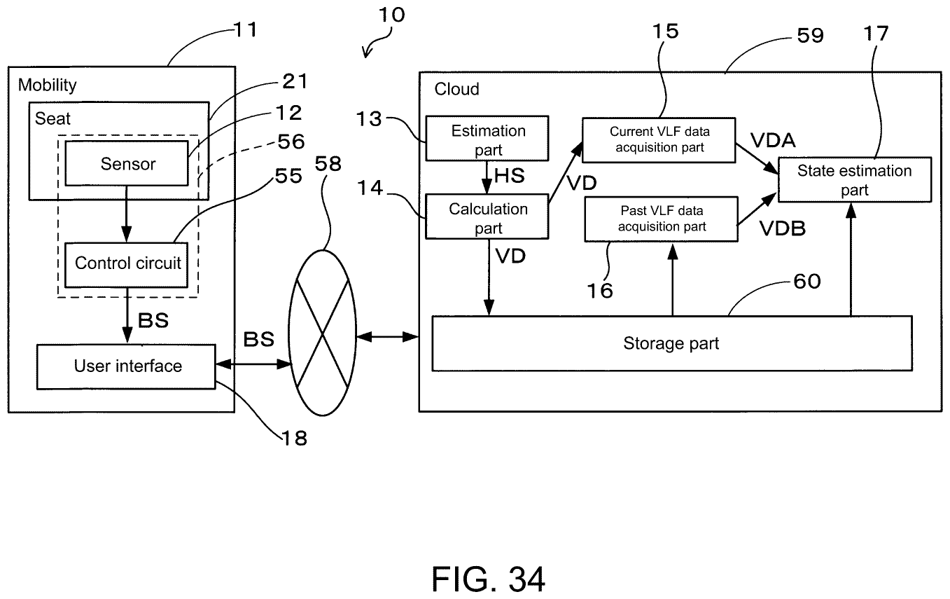

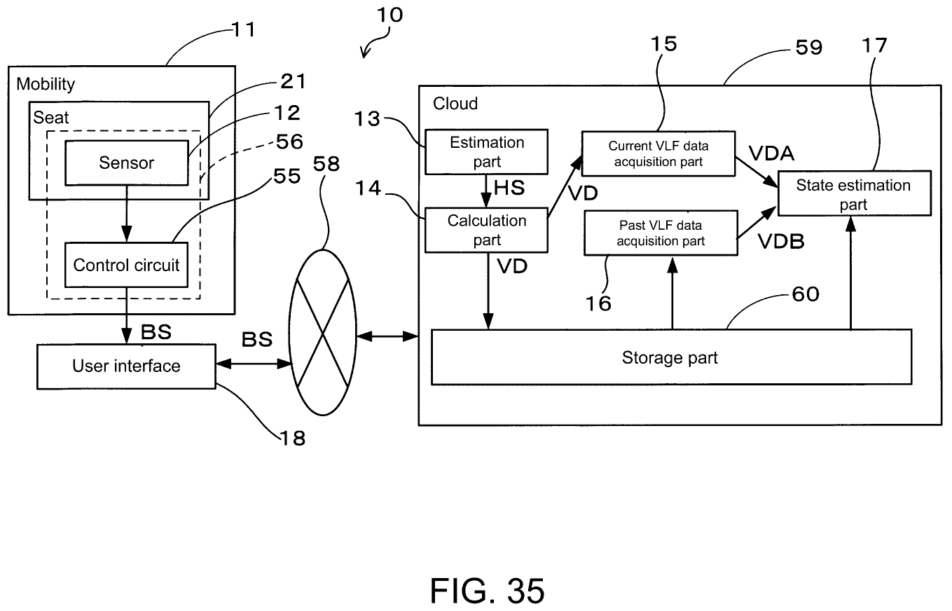

The disclosure is not limited to the above-described embodiments and may be applied to the following forms within a range that does not deviate from the spirit thereof.
(1.1) In Embodiment 1.1, the sensor 12 is configured to be attached to the seat 21 to acquire the biological signal BS from changes in body movement of the mobility user seated on the seat 21. However, the embodiment is not limited thereto, and the sensor may also emit light (infrared light, ultraviolet light, laser light, visible light, etc.) or sound to the mobility user and detect reflection from the mobility user. In addition, the sensor may also be a Doppler sensor that emits microwaves to the mobility user and detects changes in body movement of the mobility user from a difference between an emitted frequency and a received frequency, or any sensor may be appropriately selected.
(1.2) In Embodiment 1.1, the sensor 12 is configured to be attached inside the seat surface part 22a. However, the embodiment is not limited thereto, and as shown in FIG. 27, the sensor 12 may also be configured as a component separate from the seat 21 placed on the upper surface (the upper surface of the seat-surface skin member 33) of the seat surface part 22a, and attached to the seat surface part 22a by a conventional attachment means such as a rubber band or a belt. In addition, the sensor 12 may also be configured to be attached to the seat back part 22b by a conventional attachment means such as a rubber band or a belt.
(1.3) In Embodiment 1.1, the user interface 18 is a car navigation system assembled in the mobility 11. However, the embodiment is not limited thereto, and as shown in FIG. 28, the user interface 18 may also be a smartphone, a tablet terminal, a smartwatch, etc. separate from the mobility 11. In FIG. 28 to FIG. 35, illustration of the data stored in the storage part 60 is omitted. According to this embodiment, the user interface 18 may provide specific services to a mobility user who is not riding in the mobility 11. For example, a message about the health state of the mobility user may be conveyed by an image or a voice, or guidance or routes to sports facilities and the like may be displayed to the mobility user who has got off the mobility 11 and is relaxing at home.
(1.4) In Embodiment 1.1, the user interface 18 transmits or receives information to or from the cloud 59 via the network 58. However, the embodiment is not limited thereto, and as shown in FIG. 29, the configuration disposed in the cloud 59 in Embodiment 1.1 may also be assembled as a control device 62 in the mobility 11.
(1.5) In Embodiment 1.1, the user interface 18 transmits or receives information to or from the cloud 59 via the network 58. However, the embodiment is not limited thereto, and as shown in FIG. 30, the ECU 57 may be configured to transmit VLF data VD to the cloud 59 via the network 58, and the user interface 18 (e.g., a car navigation system) assembled in the mobility 11 may be configured to provide information to the mobility user.
(1.6) In Embodiment 1.1, the user interface 18 transmits or receives information to or from the cloud 59 via the network 58. However, the embodiment is not limited thereto, and as shown in FIG. 31, the ECU 57 may be configured to transmit VLF data VD to the cloud 59 via the network 58, and the user interface 18 (e.g., a smartphone, a tablet terminal, or a smartwatch) separate from the mobility 11 may be configured to provide information to the mobility user.
(1.7) In Embodiment 1.1, the ECU 57 is configured to include the calculation part 14. However, the embodiment is not limited thereto, and as shown in FIG. 32, the cloud 59 may be configured to include the calculation part 14. In that case, the ECU 57 may be configured to transmit the heart rate signal HS to the cloud 59 via the network 58, and the user interface 18 (e.g., a smartphone, a tablet terminal, or a smartwatch) separate from the mobility 11 may be configured to provide information to the mobility user.
(1.8) In Embodiment 1.1, the ECU 57 is configured to include the calculation part 14. However, the embodiment is not limited thereto, and as shown in FIG. 33, the cloud 59 may be configured to include the calculation part 14. In that case, the ECU 57 may be configured to transmit the heart rate signal HS to the cloud 59 via the network 58, and the user interface 18 (e.g., a car navigation system) assembled in the mobility 11 may be configured to provide information to the mobility user.
(1.9) In Embodiment 1.1, the mobility 11 is configured to include the estimation part 13 and the calculation part 14. However, the embodiment is not limited thereto, and as shown in FIG. 34, the cloud 59 may be configured to include the estimation part 13 and the calculation part 14.
(1.10) In Embodiment 1.1, the mobility 11 is configured to include the estimation part 13 and the calculation part 14. However, the embodiment is not limited thereto, and as shown in FIG. 35, the cloud 59 may be configured to include the estimation part 13 and the calculation part 14, and the user interface 18 (e.g., a smartphone, a tablet terminal, or a smartwatch) separate from the mobility 11 may be configured to transmit or receive information to or from the cloud 59 via the network 58.

### Embodiment 2.1

### 1. Overview of Drowsiness Information Service Providing Device 101

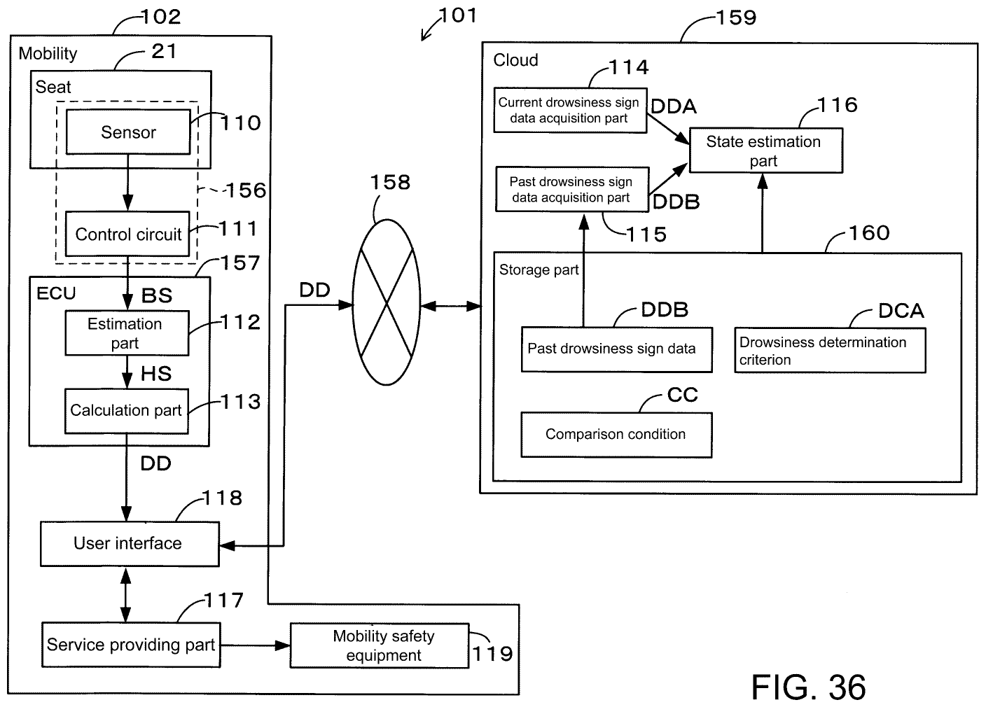

Referring to FIG. 36, an overview of a drowsiness information service providing device 101 (an example of a health information service providing device) according to Embodiment 2.1 will be described. The drowsiness information service providing device 101 of this embodiment provides, to a rider riding in a mobility 102, services related to drowsiness information associated with drowsiness of the rider. Herein, health information includes the drowsiness information.

The mobility 102 in this embodiment refers to a means of transportation that a person rides, including a vehicle such as a passenger vehicle, a cargo vehicle, a work vehicle, a train, a ship, an airplane, a helicopter, a passenger drone, etc.

As shown in FIG. 36, the drowsiness information service providing device 101 of this embodiment includes a sensor 110, a control circuit 111, an estimation part 112, a calculation part 113, a current drowsiness sign data acquisition part 114, a past drowsiness sign data acquisition part 115, a state estimation part 116, a service providing part 117, a user interface 118 (an example of providing equipment), and mobility safety equipment 119 (an example of providing equipment).

The mobility 102 includes a seat 21 where the rider sits. The sensor 110 is attached to the seat 21. In the case where the seat 21 is applied to a driver's seat, the seat 21 is used to detect a seated state of the driver and a biological signal BS of the driver during driving. In the case where the seat 21 is applied to a seat different from the driver's seat, the seat 21 is used to detect a seated state and a biological signal BS of a rider of the mobility 102 who is different from the driver.

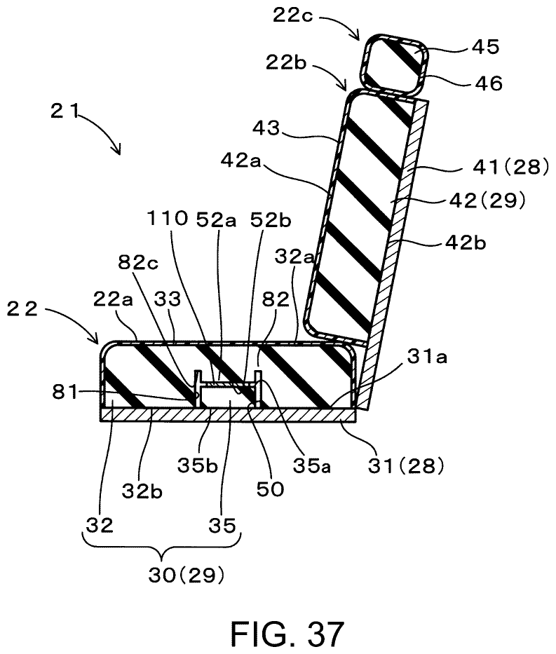

As shown in FIG. 37, the seat 21 includes a frame part 28, a cushion part 29, and a sensor 110. The seat 21 of this embodiment includes a headrest 22c. However, the headrest 22c may also be omitted.

The frame part 28 includes a seat-surface seat frame 31 and a back-surface seat frame 41. The cushion part 29 includes a seat-surface seat cushion 30 attached to the seat-surface seat frame 31, and a back-surface seat cushion 42 attached to the back-surface seat frame 41. The seat-surface seat cushion 30 includes a first seat-surface seat cushion 32 and a second seat-surface seat cushion 35.

The seat-surface seat frame 31 is formed of a rigid material such as metal or hard resin, for example, and is attached to the mobility 102. The seat-surface seat frame 31 has a plate-shaped part forming a plate shape. The plate-shaped part is attached to the mobility 102 such that a plate surface thereof faces an up-down direction. An upper surface of the plate-shaped part is taken as an attachment seat surface 31a to which the first seat-surface seat cushion 32 is attached. Portions of the seat-surface seat frame 31 other than the plate-shaped part are formed in any shape such as a rod shape, a pillar shape, etc.

The first seat-surface seat cushion 32 is formed of an elastic material such as foam resin. The first seat-surface seat cushion 32 is attached in a state of being placed on the attachment seat surface 31a formed on the upper surface of the seat-surface seat frame 31. An upper surface of the first seat-surface seat cushion 32 serves as a pressure-receiving surface 32a that receives pressure from the rider's buttocks. A lower surface of the first seat-surface seat cushion 32, i.e., a counter-pressure-receiving surface 32b which is a surface on a back side of the pressure-receiving surface 32a, is opposed to the attachment seat surface 31a of the seat-surface seat frame 31.

The first seat-surface seat cushion 32 has an accommodating recess 50 that is opened downward toward the seat-surface seat frame 31 side on the counter-pressure-receiving surface 32b. A cross-sectional shape of the accommodating recess 50 may be any shape such as a polygonal shape, a circular shape, an elliptical shape, etc. In this embodiment, the cross-sectional shape of the accommodating recess 50 is rectangular. A portion of the accommodating recess 50 that is located on a side opposite to an opening direction of the accommodating recess 50 is taken as a bottom part 52 of the accommodating recess 50.

The accommodating recess 50 accommodates the second seat-surface seat cushion 35. The second seat-surface seat cushion 35 is attached to the attachment seat surface 31a of the seat-surface seat frame 31. A surface of the second seat-surface seat cushion 35 that is opposed to the attachment seat surface 31a of the seat-surface seat frame 31 is taken as an attachment surface 35b. With the first seat-surface seat cushion 32 and the second seat-surface seat cushion 35 attached to the seat-surface seat frame 31, the counter-pressure-receiving surface 32b of the first seat-surface seat cushion 32 and the attachment surface 35b of the second seat-surface seat cushion 35 are flush with each other. In addition, with the first seat-surface seat cushion 32 and the second seat-surface seat cushion 35 attached to the seat-surface seat frame 31, a gap is formed between an inner surface of the accommodating recess 50 and an outer surface of the second seat-surface seat cushion 35. An upper surface (a surface located on a side opposite to the seat-surface seat frame 31) of the second seat-surface seat cushion 35 is taken as a second pressing surface 35a that presses the sensor 110 from below. However, the accommodating recess 50 of the first seat-surface seat cushion 32 may also be opened upward, and the second seat-surface seat cushion 35 may also be accommodated in such an accommodating recess 50.

A seat-surface skin member 33 is layered on the surface of the first seat-surface seat cushion 32. The seat-surface skin member 33 covers at least the pressure-receiving surface 32a of the first seat-surface seat cushion 32. The seat-surface skin member 33 is formed of a material that is less stretchable than the first seat-surface seat cushion 32, such as fabric or leather.

The back-surface seat frame 41 is formed of a rigid material such as metal or hard resin, for example. The back-surface seat frame 41 is formed in a plate shape, a rod shape, etc. For example, in the case of providing a reclining function to a seat body 22, the back-surface seat frame 41 is swingably supported at the seat-surface seat frame 31. Of course, the back-surface seat frame 41 may also be integrally fixed to the seat-surface seat frame 31.

The back-surface seat cushion 42 is formed of an elastic material such as foam resin. The back-surface seat cushion 42 is layered and attached to the back-surface seat frame 41. A surface of the back-surface seat cushion 42 on a side opposite to the back-surface seat frame 41 serves as a pressure-receiving surface 42a that receives pressure from the rider's back. In addition, a surface of the back-surface seat cushion 42 that is opposed to the back-surface seat frame 41 serves as a counter-pressure-receiving surface 42b of the back-surface seat cushion 42.

The surface of the back-surface seat cushion 42 is covered with a back-surface skin member 43. The back-surface skin member 43 covers at least the pressure-receiving surface 42a of the back-surface seat cushion 42. The back-surface skin member 43 is formed of a material such as fabric or leather.

The headrest 22c is disposed at an upper end of a seat back part 22b. The headrest 22c includes a cushion 45 and a skin member 46. Herein, in FIG. 37, the first seat-surface seat cushion 32 and the back-surface seat cushion 42 are separate from each other, but the first seat-surface seat cushion 32 and the back-surface seat cushion 42 may also be integrated with each other. In addition, the back-surface seat cushion 42 and the headrest 22c are separate from each other, but the back-surface seat cushion 42 and the headrest 22c may also be integrated with each other.

The sensor 110 is disposed between a first pressing surface 52b formed in the accommodating recess 50 of the first seat-surface seat cushion 32 and the second pressing surface 35a of the second seat-surface seat cushion 35. The first pressing surface 52b is provided on a protruding part 52a that protrudes downward. Herein, with a rider sitting on the seat body 22, a pressure is applied from the rider's buttocks to the pressure-receiving surface 32a of the first seat-surface seat cushion 32, and this pressure is transmitted via the first seat-surface seat cushion 32 to the first pressing surface 52b of the first seat-surface seat cushion 32. Then, the sensor 110 receives pressure from the first pressing surface 52b of the first seat-surface seat cushion 32. In other words, in the seated state of the rider, the sensor 110 detects a physical amount corresponding to the pressure transmitted via the first seat-surface seat cushion 32 from the pressure-receiving surface 32a of the first seat-surface seat cushion 32.

Herein, the sensor 110 is disposed in a seat surface part 22a, but the sensor 110 may also be disposed in the seat back part 22b. In that case, the sensor 110 is disposed between the back-surface seat frame 41 and the back-surface seat cushion 42. Then, in the seated state of the rider, the sensor 110 detects a physical amount corresponding to the pressure transmitted via the back-surface seat cushion 42 from the pressure-receiving surface 42a of the back-surface seat cushion 42.

Returning to FIG. 36, the sensor 110 is connected to a control circuit 111 for controlling the sensor 110. The control circuit 111 controls an action of the sensor 110 and acquires the physical amount detected by the sensor 110 from the sensor 110. The control circuit 111 detects a biological signal BS of the rider for the physical amount acquired from the sensor 110. The control circuit 111 may perform processing such as amplification and noise cancellation on the biological signal BS.

The sensor 110 and the control circuit 111 constitute a monitoring system 156 (an example of an acquisition device) that monitors the biological signal BS of the rider.

The control circuit 111 transmits the biological signal BS to an electrical control unit (ECU) 157. The ECU 157 includes an estimation part 112 and a calculation part 113.

The estimation part 112 acquires a biological signal BS acquired from the control circuit 111 of the monitoring system 156. The biological signal BS is a generic concept of a heart rate, a pulse rate, a body movement, a respiration, etc. of the rider, and also includes a signal in which signals of the heart rate, the pulse rate, the body movement, the respiration, etc. are mixed. The pulse rate refers to pulsation occurring in arteries. On the other hand, the heart rate refers to pulsation of the heart pumping blood throughout the body. In a healthy individual, since the heart rate and the pulse rate generally coincide with each other, hereinafter, the heart rate and the pulse rate will be collectively referred to as a heart rate signal HS related to the heart rate. The estimation part 112 estimates a heart rate signal HS related to the heart rate of the rider from the biological signal BS.

The calculation part 113 calculates drowsiness sign data DD related to a drowsiness sign of the rider based on the heart rate signal HS. The drowsiness sign data DD is a detection count of the drowsiness sign per unit time measured within a predetermined measurement time or a correlation value of the detection count. In this embodiment, based on changes in time intervals of the rider's heart rate calculated from the heart rate signal HS, it is determined whether drowsiness occurs in the rider. In the following description, the time interval of the rider's heart rate will also be simply referred to as a heartbeat interval.

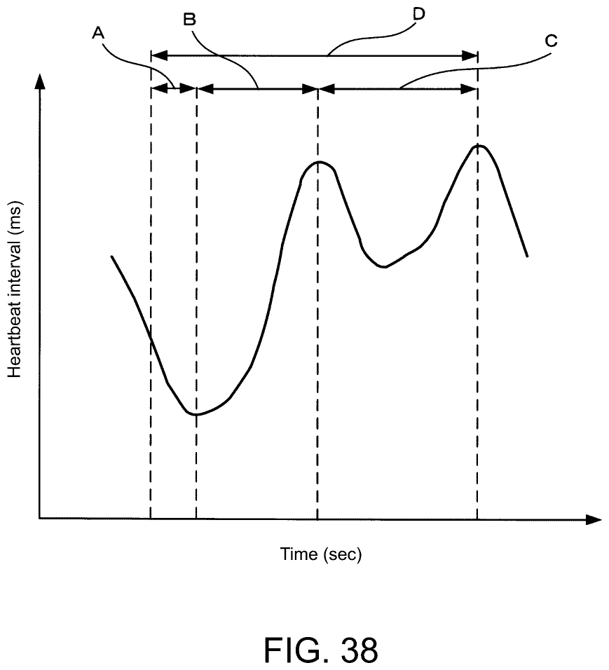

Referring to FIG. 38, the drowsiness sign data DD will be described. The graph shown in FIG. 38 shows over-time changes in heartbeat intervals. An increase in the heartbeat interval means that the rider's heart is beating slowly. A decrease in the heartbeat interval means that the rider's heart is beating quickly.

A waveform of the graph shown in FIG. 38 represents a waveform that serves as a criterion for determining the rider's drowsiness. In the graph of FIG. 38, the waveform indicated by a region D is a waveform showing an example of the criterion for determining the rider's drowsiness. The waveform related to the region D includes waveforms related to a region A to a region C. The region A is a region in which the heartbeat interval decreases, and is taken as a first period in which the graph descends. The region B occurs after the first period and is taken as a second period in which the heartbeat interval increases. The region C occurs after the second period, and the heartbeat interval decreases and then increases. In other words, in the third period, the heartbeat interval pulsates. The criterion for determining the rider's drowsiness in the graph of the heartbeat interval with respect to a measurement time is described in Japanese Patent Application Laid-Open No. 2018-192128.

In the region A, the heartbeat interval is decreasing. This corresponds to a state in which the heart rate increases as the rider tries to resist drowsiness, such as by straining the eyelids in an attempt to keep the eyes from closing. As the heart rate increases, the heartbeat becomes faster, and the heartbeat interval decreases.

In the region B, the heartbeat interval is increasing. This corresponds to a period in which drowsiness occurs again after being temporarily dispelled, and the heartbeat interval becomes longer. This is because as drowsiness occurs in the rider, the heartbeat interval increases (the heart rate decreases).

The region C corresponds to a period in which drowsiness continues.

As described above, it is possible to determine on drowsiness of the rider based on presence or absence of a waveform (region D) composed of consecutive regions including a region in which the rider consciously or unconsciously tries to resist drowsiness (corresponding to the region A) and regions in which drowsiness occurs again (region B and region C).

The drowsiness sign data DD is a detection count of the waveform corresponding to the region D in a predetermined measurement time, or a cumulative value of a calculated value calculated from the waveform corresponding to the region D in the predetermined measurement time.

Returning to FIG. 36, the calculation part 113 transmits the drowsiness sign data DD to the user interface 118 (an example of providing equipment). Examples of the user interface 118 include a car navigation system, a smartphone, a smartwatch, a dedicated terminal device for the sensor 110, etc. In this embodiment, a car navigation system installed in the mobility 102 is adopted.

The user interface 118 includes both or one of a screen display device (not shown) and a speaker (not shown) for providing specific services to the rider. The user interface 118 provides specific services to the rider by an image or a voice.

The user interface 118 includes a communication device (not shown) and is connected to a network 158 such as the Internet by the communication device. The user interface 118 transmits or receives data to or from a cloud 159 via the network 158.

The cloud 159 includes a current drowsiness sign data acquisition part 114, a past drowsiness sign data acquisition part 115, a state estimation part 116, and a storage part 160.

The current drowsiness sign data acquisition part 114 acquires current drowsiness sign data DDA from the user interface 118 via the network 158. The current drowsiness sign data DDA is defined as drowsiness sign data DD of the rider in a state of currently riding in the mobility 102.

The drowsiness sign data DD transmitted from the user interface 118 to the cloud 159 via the network 158 is acquired by the current drowsiness sign data acquisition part 114 and is sequentially stored in the storage part 160.

The past drowsiness sign data acquisition part 115 acquires past drowsiness sign data DDB stored in the storage part 160. The past drowsiness sign data DDB is drowsiness sign data DD detected when the rider rode in the mobility 102 in the past.

The state estimation part 116 estimates changes in the health state of the rider based on a difference between the current drowsiness sign data DDA and the past drowsiness sign data DDB.

The storage part 160 stores the past drowsiness sign data DDB transmitted from the user interface 118 via the network 158. In addition, the storage part 160 further stores a drowsiness determination criterion DCA and a comparison condition CC.

An estimation result related to the rider's drowsiness estimated by the state estimation part 116 is transmitted to the user interface 118 via the network 158. The estimation result related to the rider's drowsiness transmitted to the user interface 118 is transmitted to the service providing part 117. The service providing part 117 controls an action of the navigation system based on a result estimating that the rider's drowsiness has increased.

In addition, the service providing part 117 executes control on the mobility safety equipment 119 based on the result estimating that the rider's drowsiness has increased. The mobility safety equipment 119 is not particularly limited, but is a device installed on the mobility 102 for the mobility 102 to move safely. Examples of the mobility safety equipment 119 include an automatic braking system, an accelerator control system, a contact detection device for a steering wheel, a drowsiness sign detection system using an in-vehicle camera, etc.

### 2. Action of Embodiment

### 2.1. Main Routine

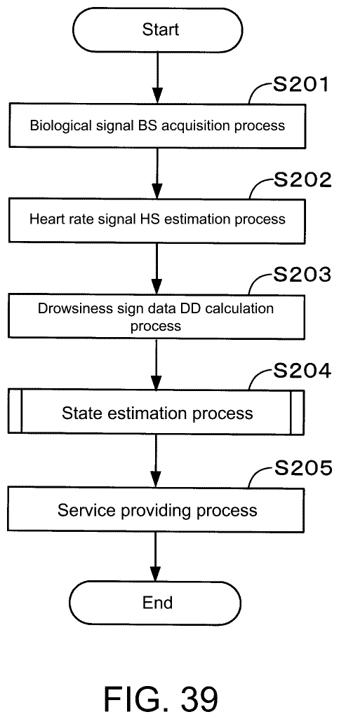

Next, referring to FIG. 39, an action of the drowsiness information service providing device 101 of this embodiment will be described. FIG. 39 shows a flowchart of the action of the drowsiness information service providing device 101 of this embodiment. Upon activation of the drowsiness information service providing device 101, a biological signal acquisition process S201 is executed. In the biological signal acquisition process S201, the sensor 110 detects a physical amount from the rider seated on the seat 21 of the mobility 102. The physical amount detected by the sensor 110 is transmitted to the control circuit 111, and the control circuit 111 calculates a biological signal BS of the rider based on the physical amount.

Next, a heart rate signal estimation process S202 is executed. In the heart rate signal estimation process S202, the estimation part 112 estimates a heart rate signal HS related to the rider's heart rate based on the biological signal BS acquired from the control circuit 111.

Next, a drowsiness sign data calculation process S203 is executed. In the drowsiness sign data calculation process S203, the calculation part 113 calculates drowsiness sign data DD based on the heart rate signal HS acquired from the estimation part 112. The calculated drowsiness sign data DD is transmitted to the cloud 159 via the network 158 from the user interface 118.

Next, a state estimation process S204 is executed. In the state estimation process S204, the state estimation part 116 estimates whether the rider's drowsiness has increased based on whether current drowsiness sign data DDA and past drowsiness sign data DDB satisfy the comparison condition CC.

Next, a service providing process S205 is executed. In the service providing process S205, the service providing part 117 executes control on the providing equipment to provide a specific service to the rider based on an estimation acquired from the state estimation part 116 that the rider's drowsiness has increased. This will be described in detail below.

The service providing part 117 may be configured to execute control on the user interface 118 which calls attention of at least one of the rider's visual sense, auditory sense, tactile sense, and olfactory sense.

In the case where a car navigation system is applied as the user interface 118, for example, services such as the follows may be provided. The user interface 118 may, for example, convey a message by a voice or by displaying on the screen to the rider, concisely informing that the rider's drowsiness has increased. The message is not particularly limited, and may be any message such as “Drowsiness is increasing.” or “How about taking a break?”.

In addition, the user interface 118 may emit a warning sound to awaken the rider.

In addition, in the case where a smartphone, a tablet terminal, or a smartwatch is applied as the user interface 118, for example, the service providing part 117 may be configured to cause the user interface 118 to vibrate to awaken the rider through the tactile sense by vibration.

The service providing part 117 may also be configured to control the mobility safety equipment 119. For example, in the case where an automatic braking system is applied as the mobility safety equipment 119, the service providing part 117 may be configured to perform control to increase a sensitivity of automatic braking to activate mitigation braking earlier.

In addition, in the case where an accelerator control system is applied as the mobility safety equipment 119, the service providing part 117 may be configured to set an upper limit on an accelerator opening, and suppress rapid acceleration of the mobility 102 even if the rider suddenly depresses the accelerator.

In addition, in the case where a contact detection device for a steering wheel is applied as the mobility safety equipment 119, a time interval for detecting contact of the rider may be shortened. Accordingly, a contact state of the rider with the steering wheel is frequently detected, and the rider is frequently warned about contact with the steering wheel. In the case where the rider does not contact the steering wheel even after receiving a warning, the service providing part 117 may be configured to promptly transition to an automatic parking mode by controlling the automatic braking system and the accelerator control system to safely park the mobility 102.

In addition, the service providing device may be configured to cause the steering wheel to vibrate to promote the rider's alertness by vibration.

In addition, in the case where a drowsiness sign detection system using an in-vehicle camera is applied as the mobility safety equipment 119, the service providing part 117 may be configured to increase a sensitivity of a camera system to make it more likely to issue a warning to the rider.

Upon ending of the service providing process S205, the action of the drowsiness information service providing device 101 is ended.

### 2.2. State Estimation Process

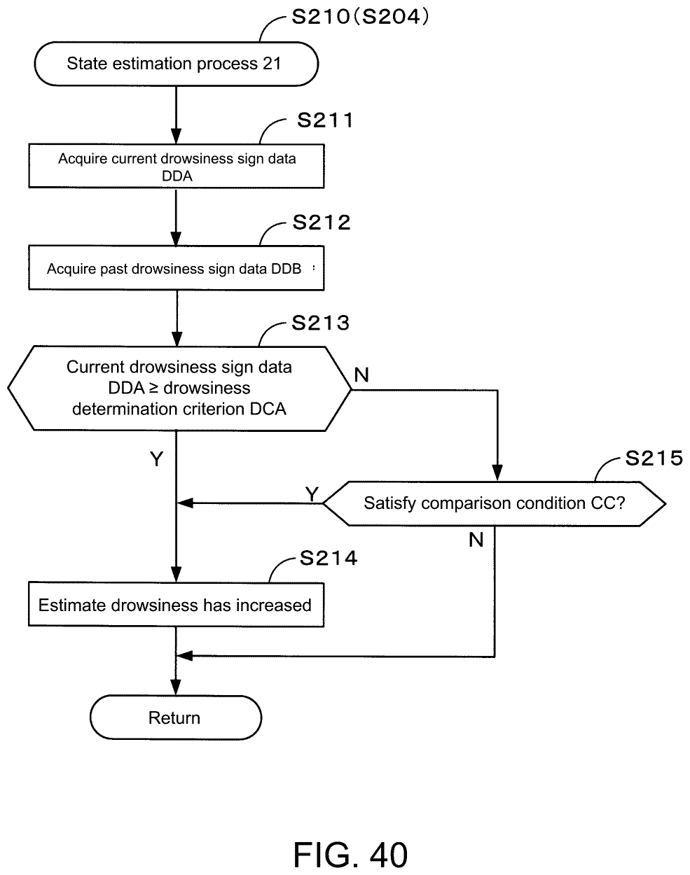

Next, referring to FIG. 40, the state estimation process S204 will be described in detail. In this embodiment, as shown in FIG. 40, a state estimation process 21 (S210) is executed as the state estimation process S204. FIG. 40 shows a flowchart of the state estimation process 21 (S210). As will be described in detail later, the state estimation process 21 of this embodiment estimates that the rider's drowsiness has increased in the case where current drowsiness sign data DDA is equal to or greater than the drowsiness determination criterion DCA. Furthermore, it is estimated that the rider's drowsiness has increased in the case where the current drowsiness sign data DDA is less than the drowsiness determination criterion DCA, and the current drowsiness sign data DDA and past drowsiness sign data DDB satisfy the comparison condition CC.

The current drowsiness sign data acquisition part 114 acquires current drowsiness sign data DDA (S211).

Next, the past drowsiness sign data acquisition part 115 acquires past drowsiness sign data DDB from the storage part 160 (S212).

Next, the state estimation part 116 determines whether the current drowsiness sign data DDA is equal to or greater than the drowsiness determination criterion DCA (S213).

When the current drowsiness sign data DDA is equal to or greater than the drowsiness determination criterion DCA (S213: Y), the state estimation part 116 estimates that the rider's drowsiness has increased (S214). Accordingly, the state estimation process 21 (S210) is ended.

In contrast, when the current drowsiness sign data DDA is less than the drowsiness determination criterion DCA (S213: N), the state estimation part 116 determines whether the current drowsiness sign data DDA and the past drowsiness sign data DDB satisfy the comparison condition CC (S215).

When the current drowsiness sign data DDA and the past drowsiness sign data DDB satisfy the comparison condition CC (S215: Y), the state estimation part 116 estimates that the rider's drowsiness has increased (S214). Accordingly, the state estimation process 21 (S210) is ended.

In contrast, when the current drowsiness sign data DDA and the past drowsiness sign data DDB do not satisfy the comparison condition CC (S215: N), the state estimation process 21 (S210) is ended.

3. Effects of this Embodiment

According to this embodiment, since whether the rider's drowsiness has increased is estimated based on whether the current drowsiness sign data DDA and the past drowsiness sign data DDB satisfy the predetermined comparison condition CC, over-time changes in the rider's drowsiness can be estimated. Accordingly, it is possible to improve accuracy of estimating changes in the rider's drowsiness compared to the case of performing determination based solely on the current drowsiness sign data DDA, which is current information of the rider.

For example, assume that in the current drowsiness sign data DDA, drowsiness sign data DD is observed 3 times in 10 minutes. On the other hand, assume that in the past drowsiness sign data DDB, drowsiness sign data DD is observed twice in 10 minutes. In that case, comparing the past drowsiness sign data DDB with the current drowsiness sign data DDA, a frequency of the drowsiness sign data DD per unit time increases. In the case where an increase in frequency between the current drowsiness sign data DDA and the past drowsiness sign data DDB as illustrated above satisfies the predetermined comparison condition CC, it can be estimated that the rider's drowsiness has increased.

In addition, in this embodiment, it is estimated that the rider's drowsiness has increased in the case where the current drowsiness sign data DDA is equal to or greater than the drowsiness determination criterion DCA, and it is estimated that the rider's drowsiness has increased in the case where the current drowsiness sign data DDA is less than the drowsiness determination criterion DCA, and the current drowsiness sign data DDA and the past drowsiness sign data DDB satisfy the comparison condition CC. Accordingly, in this embodiment, services based on two different criteria can be provided to the rider. As a result, more specialized services can be provided to the rider.

First, in the case where the current drowsiness sign data DDA is equal to or greater than the drowsiness determination criterion DCA, services based on the increase in drowsiness are provided to the rider. For example, a warning sound may be emitted, or a screen may be displayed or a voice may be emitted, informing “Drowsiness is increasing. Please take a break.” Alternatively, control may be performed to increase the sensitivity of automatic braking to activate the mitigation brake earlier, or an upper limit may be set on the accelerator opening to suppress sudden acceleration of the mobility 102 even if the rider suddenly depresses the accelerator. In addition, the contact state of the rider with the steering wheel is frequently detected, and the rider is frequently warned about contacting the steering wheel. In the case where the rider does not contact the steering wheel even after receiving a warning, the service providing part 117 may be configured to promptly transition to an automatic parking mode by controlling the automatic braking system and the accelerator control system, and safely park the mobility 102.

On the other hand, when the current drowsiness sign data DDA is less than the drowsiness determination criterion DCA, it is estimated that intense drowsiness is not occurring in the rider. However, in this embodiment, even in the above case, it is estimated that the rider's drowsiness has increased in the case where the current drowsiness sign data DDA and the past drowsiness sign data DDB satisfy the comparison condition CC. Accordingly, services based on the increase in drowsiness can be provided to the rider at a stage before intense drowsiness occurs. For example, a screen may be displayed or a voice may be emitted, informing “How about taking a break?” In addition, the sensitivity of the camera system may be increased to make it more likely to issue a warning to the rider. In addition, the steering wheel may be caused to vibrate to promote the rider's alertness by vibration.

### Embodiment 2.2

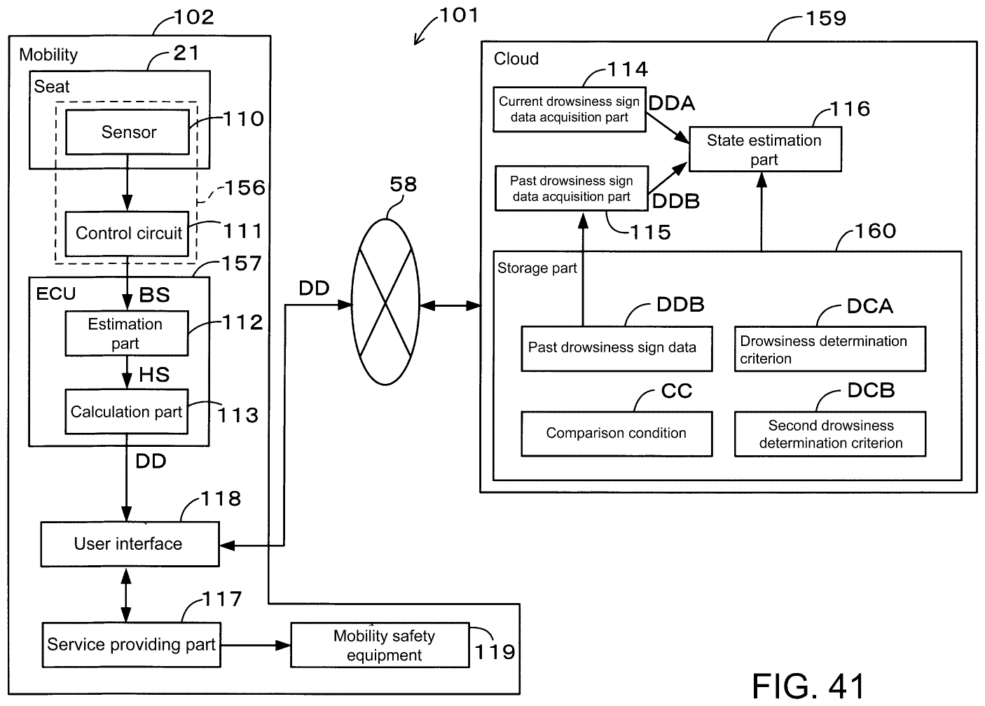

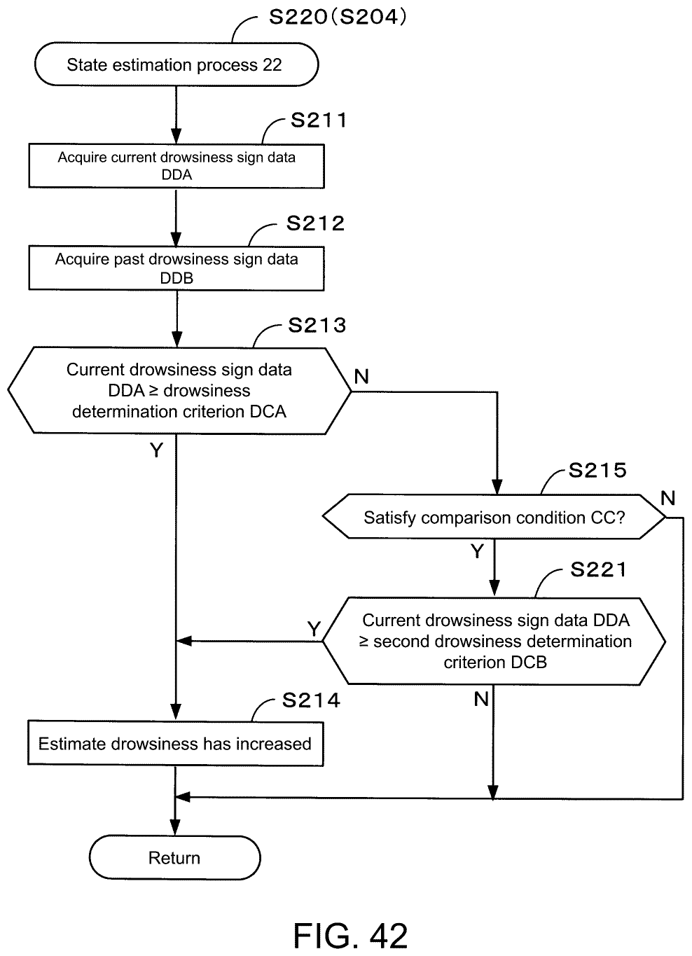

Next, referring to FIG. 41 to FIG. 42, Embodiment 2.2 will be described. As shown in FIG. 41, this embodiment differs from Embodiment 2.1 in that the storage part 160 stores a second drowsiness determination criterion DCB.

In addition, this embodiment differs from Embodiment 2.1 in that a state estimation process 22 (S220) is executed instead of the state estimation process 21 (S210) (refer to FIG. 42). Among reference signs used from Embodiment 2.2 onward, reference signs that are identical to those used in previous embodiments represent constituent elements and the like similar to those in the previous embodiments unless otherwise specified.

Next, referring to FIG. 42, the state estimation process 22 (S220) will be described. The state estimation process 22 (S220) of this embodiment differs from the state estimation process 21 (S210) in FIG. 40 in that a step (S221) is executed to determine whether the current drowsiness sign data DDA is equal to or greater than the second drowsiness determination criterion DCB. The same processes as those in the state estimation process 21 (S210) will be labeled with the same reference signs, and repeated descriptions thereof will be omitted.

As shown in FIG. 42, when the current drowsiness sign data DDA and the past drowsiness sign data DDB satisfy the comparison condition CC (S215: Y), the state estimation part 116 determines whether the current drowsiness sign data DDA is equal to or greater than the second drowsiness determination criterion DCB stored in the storage part 160 (S221).

When the current drowsiness sign data DDA is equal to or greater than the second drowsiness determination criterion DCB (S221: Y), the state estimation part 116 estimates that the rider's drowsiness has increased (S214). Accordingly, the state estimation process 22 (S220) is ended.

When the current drowsiness sign data DDA is less than the second drowsiness determination criterion DCB (S221: N), the state estimation process 22 (S220) is ended.

According to this embodiment, in the case where the current drowsiness sign data DDA is less than the drowsiness determination criterion DCA, and the current drowsiness sign data DDA and the past drowsiness sign data DDB satisfy the comparison condition CC, the current drowsiness sign data DDA is compared with the second drowsiness determination criterion DCB. Since the second drowsiness determination criterion DCB is smaller than the drowsiness determination criterion DCA, it becomes more likely to estimate that the rider's drowsiness has increased. Accordingly, even in the case of estimating that the rider's drowsiness has not increased based on the drowsiness determination criterion DCA, if the comparison condition CC is satisfied upon relatively comparing the current drowsiness sign data DDA and the past drowsiness sign data DDB, it becomes possible to estimate changes in the rider's drowsiness at a level lower than the drowsiness determination criterion DCA to be more likely to estimate that the rider's drowsiness has increased. As a result, it is possible to improve safety of the rider during driving of the mobility 102.

### Embodiment 2.3

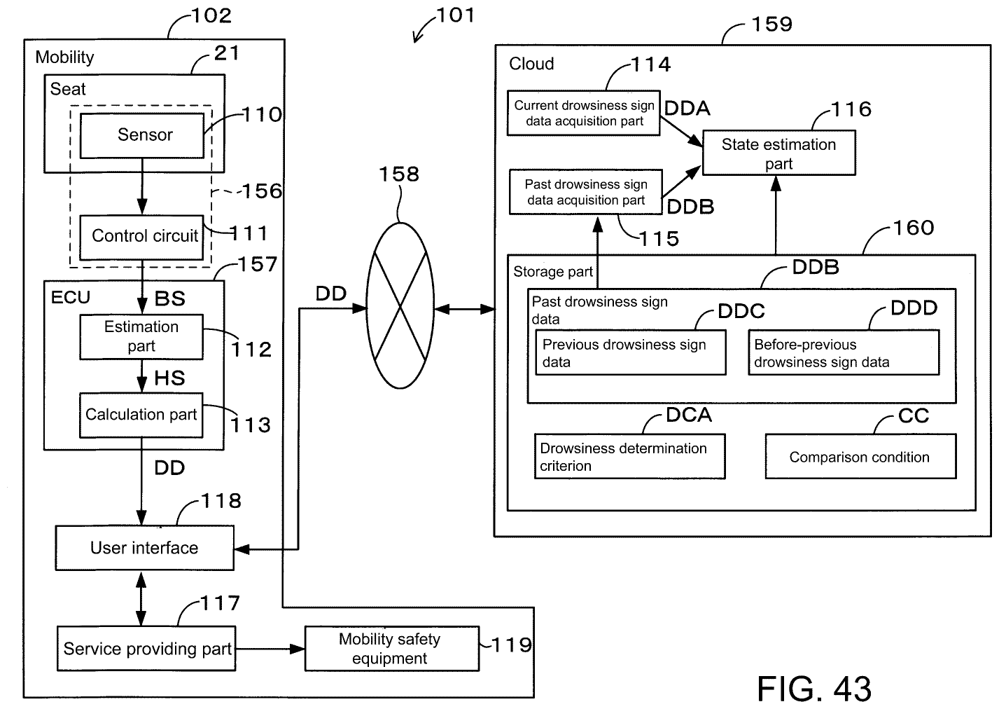

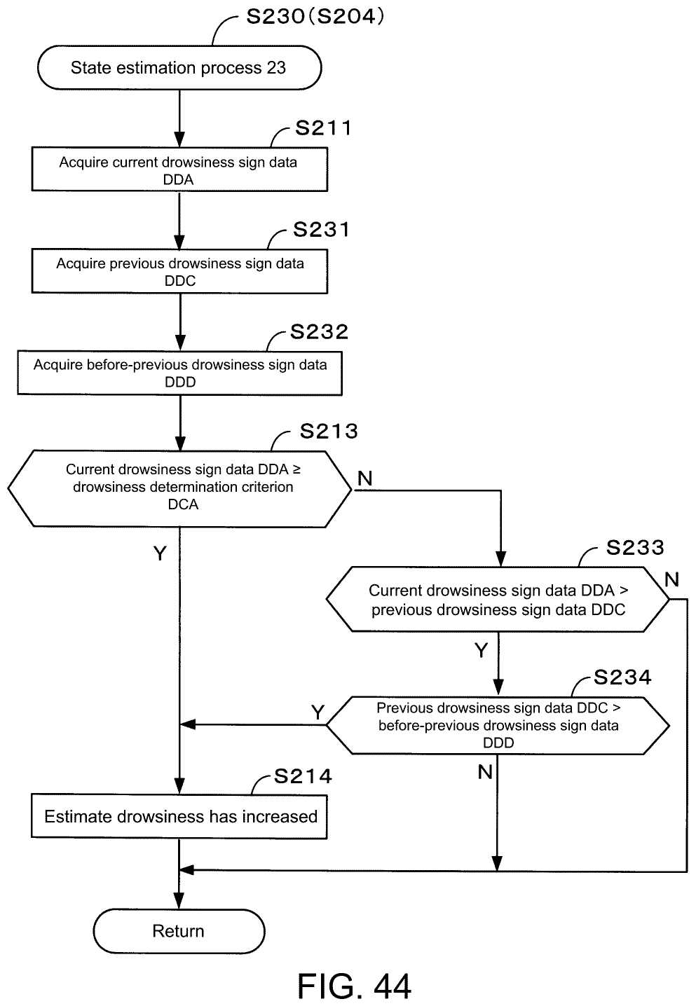

Next, referring to FIG. 43 to FIG. 44, Embodiment 2.3 will be described. As shown in FIG. 43, this embodiment differs from Embodiment 2.1 in that the past drowsiness sign data DDB includes previous drowsiness sign data DDC and before-previous drowsiness sign data DDD.

In addition, this embodiment differs from Embodiment 2.1 in that a state estimation process 23 (S230) is executed instead of the state estimation process 21 (S210) (refer to FIG. 44).

Next, referring to FIG. 44, the state estimation process 23 (S230) will be described. Upon execution of the state estimation process 23 (S230), the current drowsiness sign data acquisition part 114 acquires current drowsiness sign data DDA (S211).

Next, the past drowsiness sign data acquisition part 115 acquires previous drowsiness sign data DDC from the storage part 160 (S231). The previous drowsiness sign data DDC is drowsiness sign data DD calculated based on a heart rate signal HS when the rider rode in the mobility 102 last time.

Next, the past drowsiness sign data acquisition part 115 acquires the before-previous drowsiness sign data DDD from the storage part 160 (S232). The before-previous drowsiness sign data DDD is drowsiness sign data DD calculated based on a heart rate signal HS when the rider rode in the mobility 102 the time immediately before last time.

Next, the state estimation part 116 determines whether the current drowsiness sign data DDA is equal to or greater than the drowsiness determination criterion DCA (S213).

When the current drowsiness sign data DDA is equal to or greater than the drowsiness determination criterion DCA (S213: Y), the state estimation part 116 estimates that the rider's drowsiness has increased (S214). Accordingly, the state estimation process 23 (S230) is ended.

In contrast, when the current drowsiness sign data DDA is less than the drowsiness determination criterion DCA (S213: N), the state estimation part 116 determines whether the current drowsiness sign data DDA is greater than the previous drowsiness sign data DDC (S233).

When the current drowsiness sign data DDA is greater than the previous drowsiness sign data DDC (S233: Y), the state estimation part 116 determines whether the previous drowsiness sign data DDC is greater than the before-previous drowsiness sign data DDD (S234).

When the previous drowsiness sign data DDC is greater than the before-previous drowsiness sign data DDD (S234: Y), the state estimation part 116 estimates that the rider's drowsiness has increased (S214). Accordingly, the state estimation process 23 (S230) is ended. In this manner, the state estimation part 116 estimates that the rider's drowsiness has increased when the current drowsiness sign data DDA is greater than the previous drowsiness sign data DDC, and the previous drowsiness sign data DDC is greater than the before-previous drowsiness sign data DDD. In other words, the comparison condition CC in this embodiment is that the current drowsiness sign data DDA is greater than the previous drowsiness sign data DDC, and the previous drowsiness sign data DDC is greater than the before-previous drowsiness sign data DDD.

In contrast, when the current drowsiness sign data DDA is equal to or less than the previous drowsiness sign data DDC (S233: N), the state estimation process 23 (S230) is ended.

In addition, when the previous drowsiness sign data DDC is equal to or less than the before-previous drowsiness sign data DDD (S234: N), the state estimation process 23 (S230) is ended.

According to this embodiment, in the case where the current drowsiness sign data DDA is less than the drowsiness determination criterion DCA, and the current drowsiness sign data DDA, the previous drowsiness sign data DDC, and the before-previous drowsiness sign data DDD satisfy the comparison condition CC, it is estimated that the rider's drowsiness has increased. As described above, the comparison condition CC in this embodiment is that the current drowsiness sign data DDA is greater than the previous drowsiness sign data DDC, and the previous drowsiness sign data DDC is greater than the before-previous drowsiness sign data DDD. In this manner, by using the previous drowsiness sign data DDC and the before-previous drowsiness sign data DDD, it is possible to perform drowsiness determination of the rider more precisely.

### Embodiment 2.4

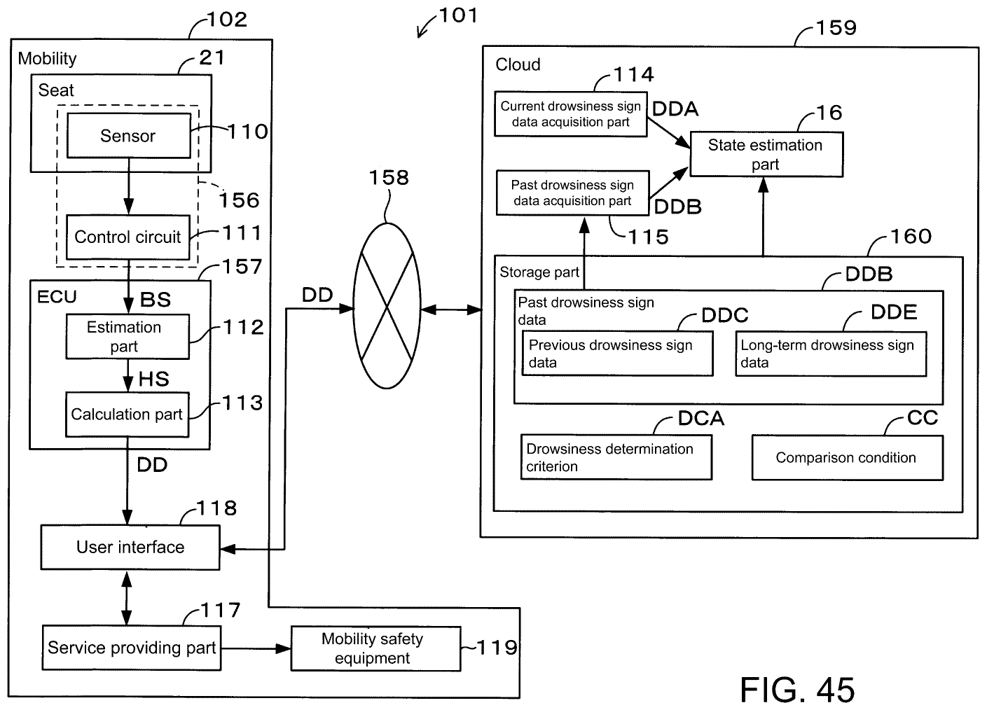

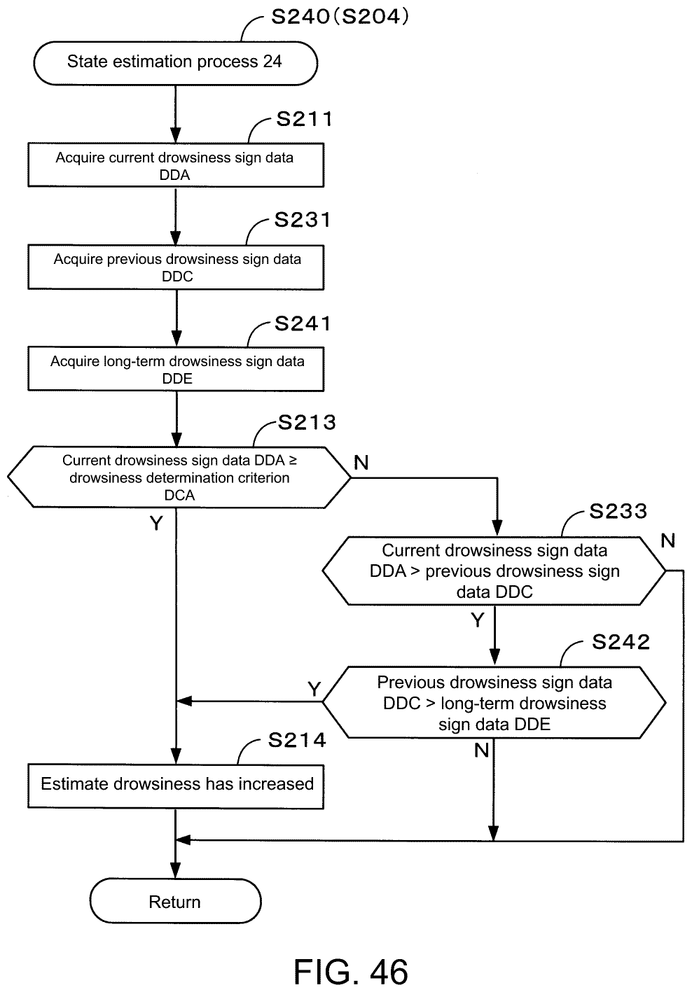

Next, referring to FIG. 45 to FIG. 46, Embodiment 2.4 will be described. As shown in FIG. 45, this embodiment differs from Embodiment 2.3 in that the past drowsiness sign data DDB includes long-term drowsiness sign data DDE instead of the before-previous drowsiness sign data DDD.

In addition, this embodiment differs from Embodiment 2.3 in that a state estimation process 24 (S240) is executed instead of the state estimation process 23 (S230) (FIG. 46).

FIG. 46 shows a flowchart of the state estimation process 24 (S240). The state estimation process 24 (S240) differs from Embodiment 2.3 in that the past drowsiness sign data acquisition part 115 executes a process to acquire long-term drowsiness sign data DDE (S241), and the state estimation part 116 determines whether the previous drowsiness sign data DDC is greater than the long-term drowsiness sign data DDE (S242).

As shown in FIG. 46, upon execution of the state estimation process 24 (S240), S211 and S231 are executed. Since S211 and S231 are the same processes as those in Embodiment 2.3, repeated descriptions thereof will be omitted.

The past drowsiness sign data acquisition part 115 acquires long-term drowsiness sign data DDE (S241). The long-term drowsiness sign data DDE is drowsiness sign data DD per unit time calculated based on heart rate signals HS when the rider rode in the mobility 102 multiple times in the past.

Subsequently, the state estimation part 116 determines whether the current drowsiness sign data DDA is equal to or greater than the drowsiness determination criterion DCA (S213).

When the current drowsiness sign data DDA is equal to or greater than the drowsiness determination criterion DCA (S213: Y), the state estimation part 116 estimates that the rider's drowsiness has increased (S214). Accordingly, the state estimation process 24 (S240) is ended.

In contrast, when the current drowsiness sign data DDA is smaller than the drowsiness determination criterion DCA (S213: N), the state estimation part determines whether the current drowsiness sign data DDA is greater than the previous drowsiness sign data DDC (S233).

When the current drowsiness sign data DDA is greater than the previous drowsiness sign data DDC (S233: Y), the state estimation part 116 determines whether the previous drowsiness sign data DDC is greater than the long-term drowsiness sign data DDE (S242).

When the previous drowsiness sign data DDC is greater than the long-term drowsiness sign data DDE (S242: Y), the state estimation part 116 estimates that the rider's drowsiness has increased (S214). Accordingly, the state estimation process 24 (S240) is ended. In this manner, the state estimation part 116 estimates that the rider's drowsiness has increased when the current drowsiness sign data DDA is greater than the previous drowsiness sign data DDC, and the previous drowsiness sign data DDC is greater than the long-term drowsiness sign data DDE. In other words, the comparison condition CC in this embodiment is that the current drowsiness sign data DDA is greater than the previous drowsiness sign data DDC, and the previous drowsiness sign data DDC is greater than the long-term drowsiness sign data DDE.

In contrast, when the current drowsiness sign data is equal to or less than the previous drowsiness sign data DDC (S233: N), the state estimation process 24 (S240) is ended.

In addition, when the previous drowsiness sign data DDC is equal to or less than the long-term drowsiness sign data DDE (S242: N), the state estimation process 24 (S240) is ended.

According to this embodiment, in the case where the current drowsiness sign data DDA is less than the drowsiness determination criterion DCA, and the current drowsiness sign data DDA, the previous drowsiness sign data DDC, and the long-term drowsiness sign data DDE satisfy the comparison condition CC, it is estimated that the rider's drowsiness has increased. As described above, the comparison condition CC in this embodiment is that the current drowsiness sign data DDA is greater than the previous drowsiness sign data DDC, and the previous drowsiness sign data DDC is greater than the long-term drowsiness sign data DDE. In this manner, by using the long-term drowsiness sign data DDE when estimating whether the rider's drowsiness has increased, it is possible to estimate the rider's drowsiness considering the rider's health state over a relatively long term. This will be described in detail below.

For example, assume that the drowsiness determination criterion DCA is set to estimate that the rider's drowsiness has increased in the case where drowsiness sign data DD is observed 5 times in 10 minutes. In that case, assume that, for example, in the current drowsiness sign data DDA, a state in which drowsiness sign data DD is observed 3 times in 10 minutes continues. Thus, based on the drowsiness determination criterion DCA, it is not estimated that the rider's drowsiness has increased.

However, assume that, for example, in the past drowsiness sign data DDB one week ago, drowsiness sign data DD is observed twice in 10 minutes. In that case, from last week to this week, an observation count of the drowsiness sign data DD in 10 minutes has increased from twice to 3 times. According to this embodiment, the state estimation part 116 can estimate that the rider's drowsiness has increased. Accordingly, it becomes possible to accurately estimate the rider's drowsiness that cannot be estimated based on the drowsiness determination criterion DCA. However, the drowsiness determination criterion DCA is not limited to the above value.

### Embodiment 2.5

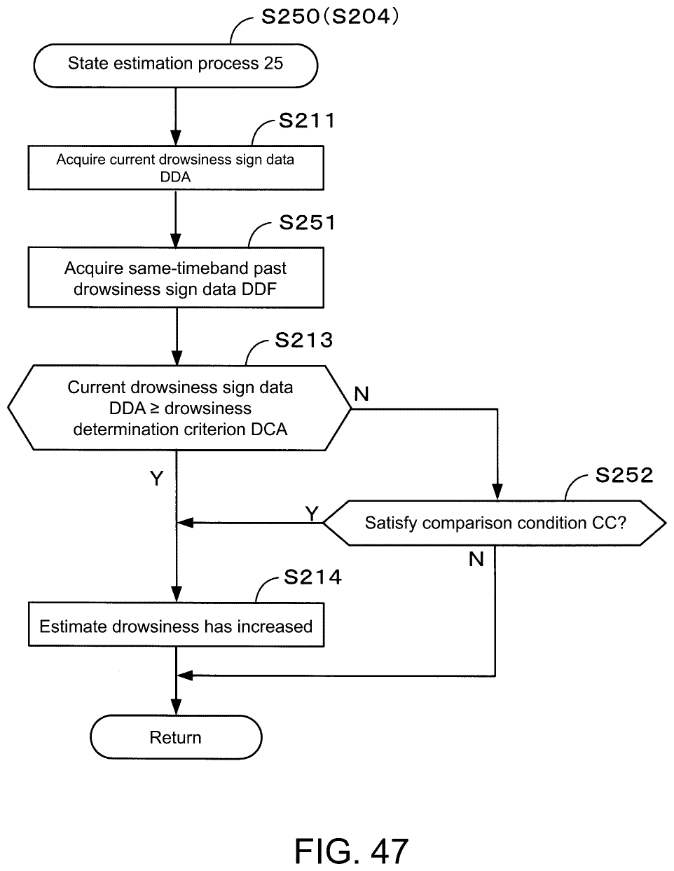

Next, referring to FIG. 47, Embodiment 2.5 will be described. This embodiment differs from Embodiment 2.1 in that a state estimation process 25 (S250) is executed instead of the state estimation process 21 (S210).

The state estimation process 25 (S250) differs from the state estimation process 21 (S210) in that S251 is executed instead of S212 in FIG. 40, and S252 is executed instead of S215. The same processes as those in the state estimation process 21 (S210) in FIG. 40 will be labeled with the same reference signs, and repeated descriptions thereof will be omitted.

The past drowsiness sign data acquisition part 115 acquires same-timeband past drowsiness sign data DDF from the storage part 160 (S251). The same-timeband past drowsiness sign data DDF is drowsiness sign data DD calculated based on a heart rate signal HS when the rider rode in the mobility 102 in a past timeband including a time point at which the current drowsiness sign data DDA is measured.

The same-timeband past drowsiness sign data DDF will be described. For example, suppose a case where a rider using the mobility 102 commutes by riding in the mobility 102 every day from 7:30 to 8:30 on weekdays from Monday to Friday. For example, in the case where the current drowsiness sign data DDA is acquired during riding at 8:00 in the morning on Friday, the past drowsiness sign data DDB from 7:30 to 8:30 on Thursday (the previous day) is illustrated as the same-timeband past drowsiness sign data DDF. In the above example, the same-timeband past drowsiness sign data DDF is not limited to the Thursday (the previous day), but may also be drowsiness sign data DD measured during riding in the mobility 102 in a timeband including 8:00 in the morning prior to the Friday on which the current drowsiness sign data DDA is measured. However, a measurement time of the current drowsiness sign data DDA is any measurement time and is not limited to the above-described timeband.

In this embodiment, in S252, the state estimation part 116 determines whether the current drowsiness sign data DDA and the same-timeband past drowsiness sign data DDF satisfy the comparison condition CC. The same-timeband past drowsiness sign data DDF is calculated in a past timeband including the time point at which the current drowsiness sign data DDA is measured. Thus, a measurement condition of the same-timeband past drowsiness sign data DDF is relatively similar compared to past drowsiness sign data DDB measured in a timeband different from the measurement time point of the current drowsiness sign data DDA. By relatively comparing the same-timeband past drowsiness sign data DDF, which is measured under relatively similar conditions, with the current drowsiness sign data DDA, over-time changes in the rider's drowsiness from the past to the present can be estimated with high accuracy.

### Embodiment 2.6

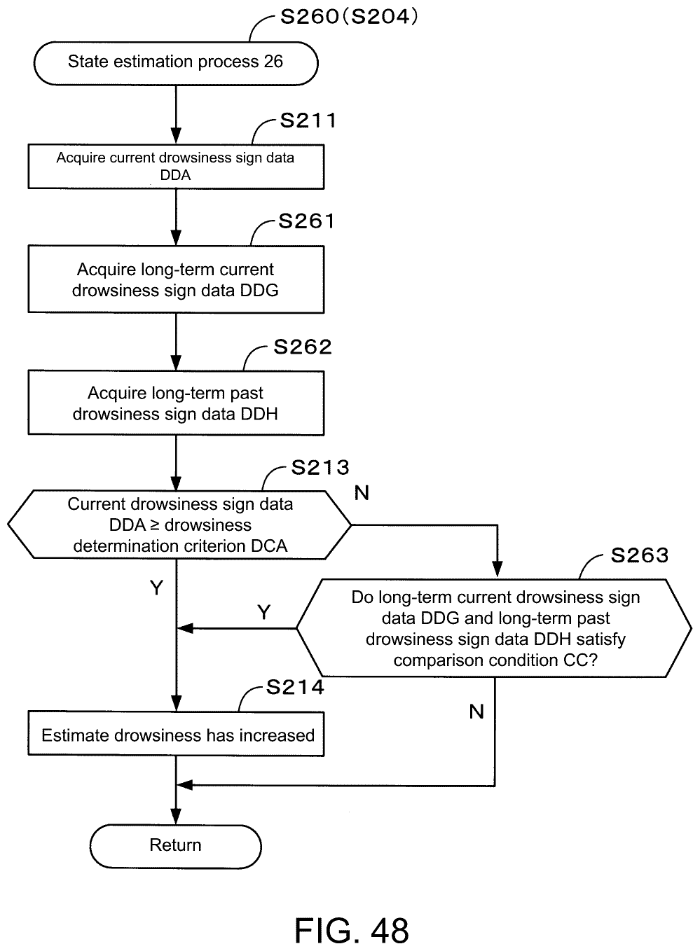

Next, referring to FIG. 48, Embodiment 2.6 will be described. This embodiment differs from Embodiment 2.1 in that a state estimation process 26 (S260) is executed instead of the state estimation process 21 (S210) in FIG. 40.

FIG. 48 shows a flowchart of the state estimation process 26 (S260). Upon execution of the state estimation process 26 (S260), the current drowsiness sign data acquisition part 114 acquires current drowsiness sign data DDA (S211).

Next, the current drowsiness sign data acquisition part 114 acquires long-term current drowsiness sign data DDG (S261). The long-term current drowsiness sign data DDG is drowsiness sign data DD calculated based on a heart rate signal HS in a predetermined comparison period including the present time. The comparison period is a period longer than a minimum time when acquiring the current drowsiness sign data DDA. For example, any period may be appropriately selected as the comparison period, such as a one-time riding duration, one day, one week, one month, etc. The period may be selected by the rider or may be set in advance.

Next, the past drowsiness sign data acquisition part 115 acquires long-term past drowsiness sign data DDH from the storage part 160 (S262). The long-term past drowsiness sign data DDH is drowsiness sign data DD calculated based on a heart rate signal HS in a comparison period prior to the present time. Since the comparison period is similar to that in the long-term current drowsiness sign data DDG, repeated descriptions thereof will be omitted. In this embodiment, the comparison period related to the long-term current drowsiness sign data DDG and the comparison period related to the long-term past drowsiness sign data DDH are the same.

Next, the state estimation part 116 determines whether the current drowsiness sign data DDA is equal to or greater than the drowsiness determination criterion DCA (S213).

When the current drowsiness sign data DDA is equal to or greater than the drowsiness determination criterion DCA (S213: Y), the state estimation part 116 estimates that the rider's drowsiness has increased (S214). Accordingly, the state estimation process 26 (S260) is ended.

In contrast, when the current drowsiness sign data DDA is smaller than the drowsiness determination criterion DCA (S213: N), the state estimation part 116 determines whether the long-term current drowsiness sign data DDG and the long-term past drowsiness sign data DDH satisfy the comparison condition CC (S263).

When the long-term current drowsiness sign data DDG and the long-term past drowsiness sign data DDH satisfy the comparison condition CC (S263: Y), the state estimation part 116 estimates that the rider's drowsiness has increased (S214). Accordingly, the state estimation process 26 (S260) is ended.

In contrast, when the long-term current drowsiness sign data DDG and the long-term past drowsiness sign data DDH do not satisfy the comparison condition CC (S263: N), the state estimation process 26 (S260) is ended.

According to this embodiment, in the case where the current drowsiness sign data DDA is smaller than the drowsiness determination criterion DCA and it is not estimated that the rider's drowsiness has increased, the long-term current drowsiness sign data DDG and the long-term past drowsiness sign data DDH are relatively compared, and it is estimated whether the rider's drowsiness has increased based on whether the comparison condition CC is satisfied. By comparing the long-term current drowsiness sign data DDG and the long-term past drowsiness sign data DDH, it is possible to compare the current drowsiness sign data DDA and the past drowsiness sign data DDB in a relatively long period, so over-time changes in the rider's drowsiness can be estimated from a long-term perspective.

For example, taking the comparison period as a one-time riding duration, it is possible to estimate changes in drowsiness for each one-time riding duration. Similarly, it is possible to estimate changes in the rider's drowsiness in the riding period for each day, each week, or each month. In addition, by setting any period, it is possible to estimate changes in drowsiness for each shift period of work for a rider involved in transportation work, for example. In this manner, changes in the rider's drowsiness can be estimated from a long-term perspective.

### Embodiment 2.7

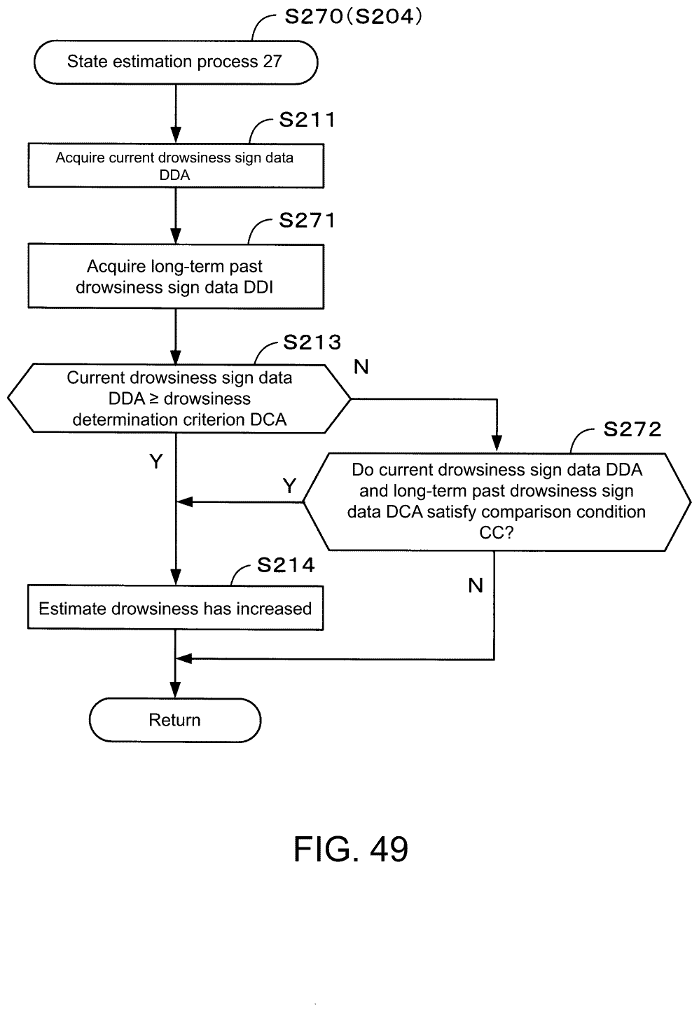

Next, referring to FIG. 49, Embodiment 2.7 will be described. This embodiment differs from Embodiment 2.1 in that a state estimation process 27 (S270) is executed instead of the state estimation process 21 (S210) in FIG. 40.

FIG. 49 shows a flowchart of the state estimation process 27 (S270). Upon execution of the state estimation process 27 (S270), the current drowsiness sign data acquisition part 114 acquires current drowsiness sign data DDA (S211).

Next, the past drowsiness sign data acquisition part 115 acquires long-term past drowsiness sign data DDI from the storage part 160 (S271). The long-term past drowsiness sign data DDI is a detection count of a drowsiness sign per unit time or a correlation value of the detection count, calculated based on a heart rate signal HS in a predetermined period which is a time longer than a period of the heart rate signal HS used to calculate the current drowsiness sign data DDA. Any period may be selected as the predetermined period, such as a one-time riding duration, one day, one week, one month, etc.

Next, the state estimation part 116 determines whether the current drowsiness sign data DDA is equal to or greater than the drowsiness determination criterion DCA (S213).

When the current drowsiness sign data DDA is equal to or greater than the drowsiness determination criterion DCA (S213: Y), the state estimation part 116 estimates that the rider's drowsiness has increased (S214). Accordingly, the state estimation process 27 (S270) is ended.

In contrast, when the current drowsiness sign data DDA is smaller than the drowsiness determination criterion DCA (S213: N), the state estimation part 116 determines whether the current drowsiness sign data DDA and the long-term past drowsiness sign data DDI satisfy the comparison condition CC (S272).

When the current drowsiness sign data DDA and the long-term past drowsiness sign data DDI satisfy the comparison condition CC (S272: Y), the state estimation part 116 estimates that the rider's drowsiness has increased (S214). Accordingly, the state estimation process 27 (S270) is ended.

In contrast, when the current drowsiness sign data DDA and the long-term past drowsiness sign data DDI do not satisfy the comparison condition CC (S272: N), the state estimation process 27 (S270) is ended.

According to this embodiment, it is estimated whether the rider's drowsiness has increased based on whether the current drowsiness sign data DDA and the long-term drowsiness sign data, which is measured in a predetermined period that is a time longer than the current drowsiness sign data DDA, satisfy the comparison condition CC. For example, suppose that the predetermined period is set to one week. In that case, suppose that, in the current drowsiness sign data DDA of the rider, drowsiness sign data DD is observed 3 times in 10 minutes. On the other hand, in the long-term past drowsiness sign data DDI, suppose that, when the unit time is taken as 10 minutes, drowsiness sign data DD is observed twice in 10 minutes. As a result, it is learned that the rider's drowsiness has changed in an increasing direction compared to the last week. Accordingly, it is possible to estimate changes in the rider's drowsiness based on over-time changes in the rider's drowsiness over a long time.

### Embodiment 2.8

Next, Embodiment 2.8 will be described. In this embodiment, the service providing part 117 executes control on the providing equipment to advance a timing of providing a service to the rider in the case where it is estimated that the rider's drowsiness has increased. Accordingly, it is possible to provide a service that promptly responds to changes in the rider's drowsiness. Since other aspects are substantially similar to Embodiment 2.1, the same members will be labeled with the same reference signs, and repeated descriptions thereof will be omitted. The following description refers to FIG. 40.

For example, assume that the drowsiness determination criterion DCA is set to estimate that the rider's drowsiness has increased in the case where drowsiness sign data DD is observed 5 times in 10 minutes. In that case, for example, assume that, in the current drowsiness sign data DDA, a state in which drowsiness sign data DD is observed 3 times in 10 minutes continues. Thus, based on the drowsiness determination criterion DCA, it is not estimated that the rider's drowsiness has increased (S213: N).

However, in the case where the current drowsiness sign data DDA and the past drowsiness sign data DDB satisfy the comparison condition CC (S215: Y), it is estimated that the rider's drowsiness has increased (S214).

Then, the service providing part 117 reduces the value of the drowsiness determination criterion DCA, for example. Accordingly, it becomes more likely for the state estimation part 116 to estimate the rider's drowsiness. As a result, compared to the state before the drowsiness determination criterion DCA is reduced, a timing for the providing device to provide a service to the rider becomes earlier. Consequently, for example, since a message that drowsiness has increased can be promptly provided to the rider by screen display or a voice, safety of the mobility 102 can be improved. However, the drowsiness determination criterion DCA is not limited to the above value.

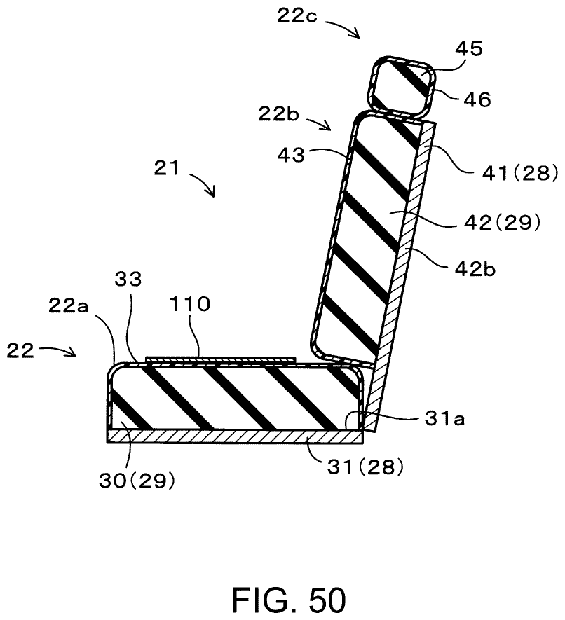

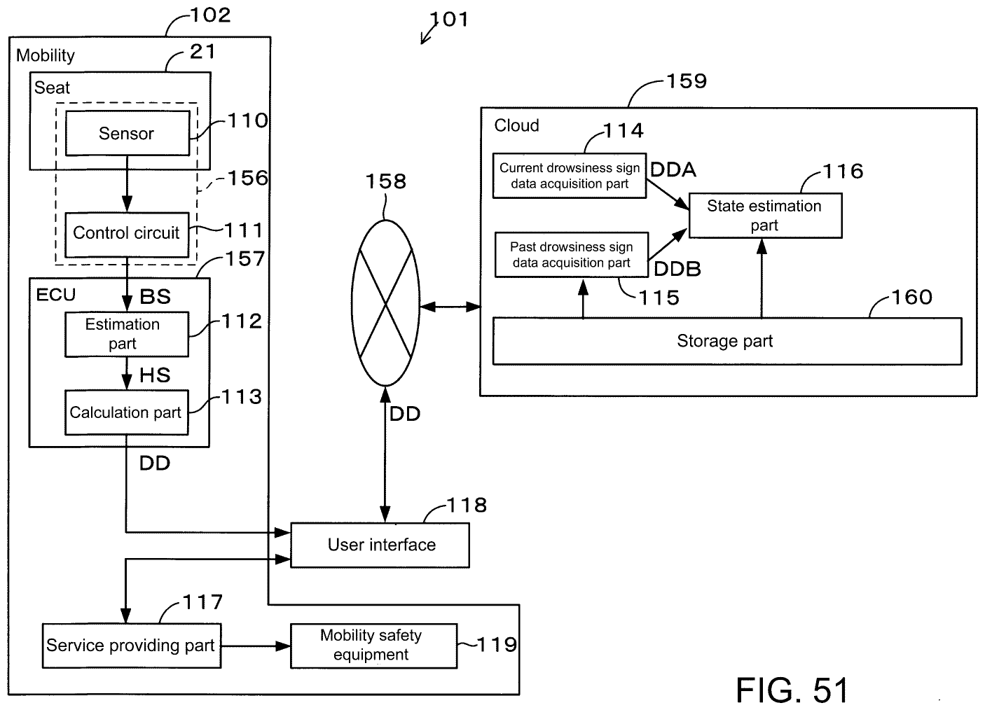

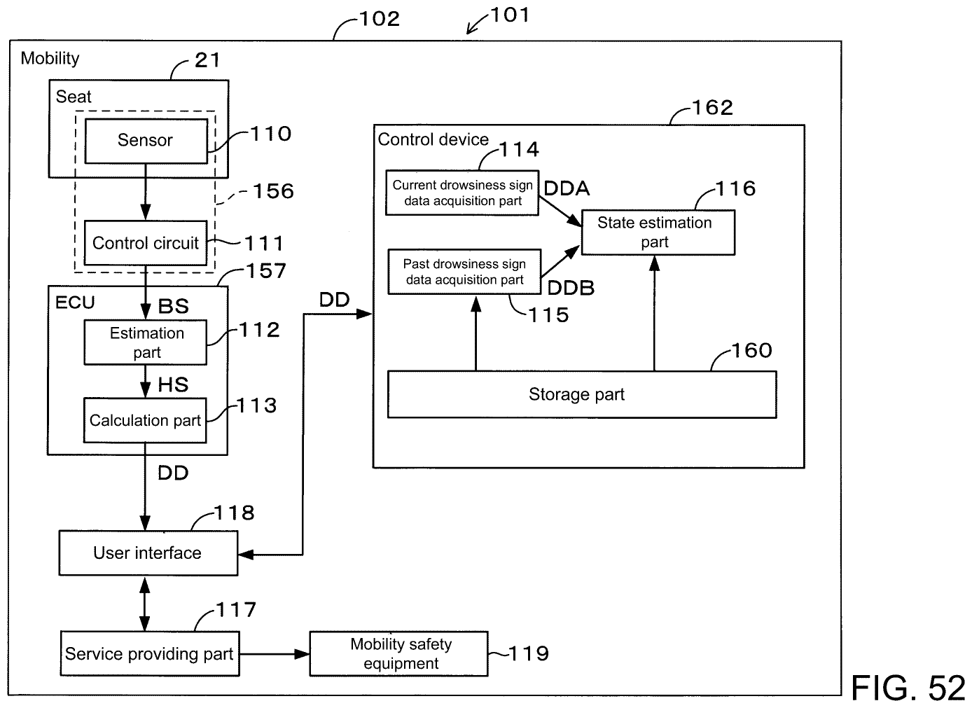

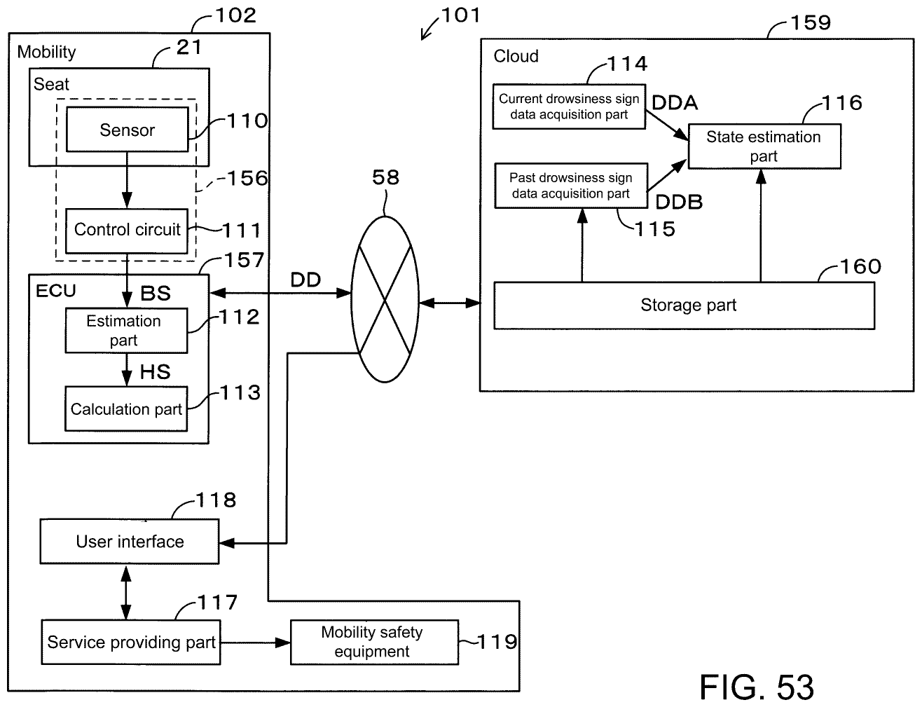

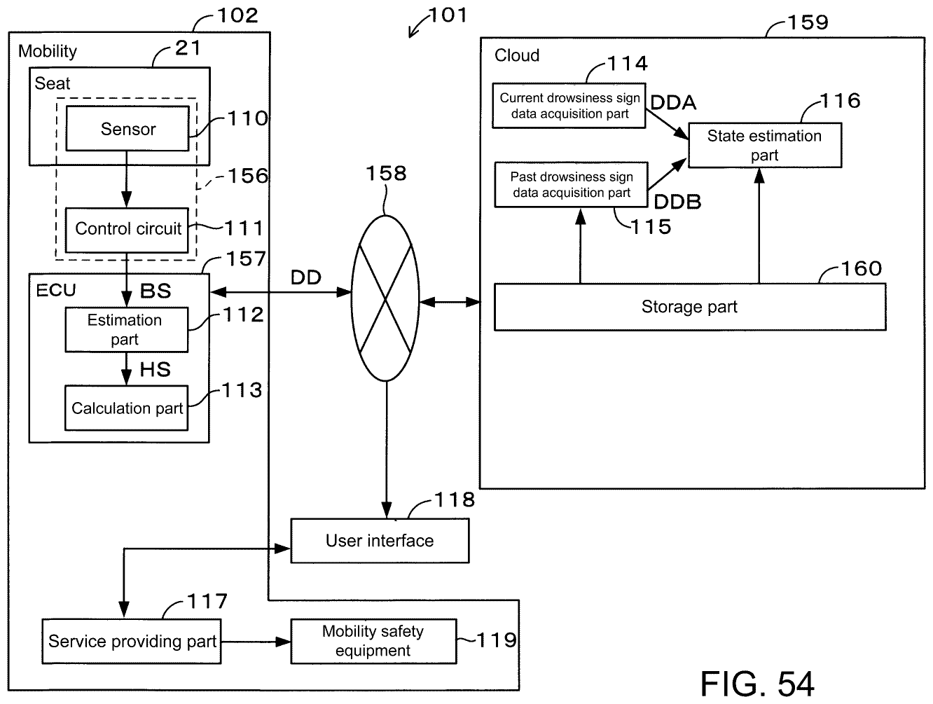

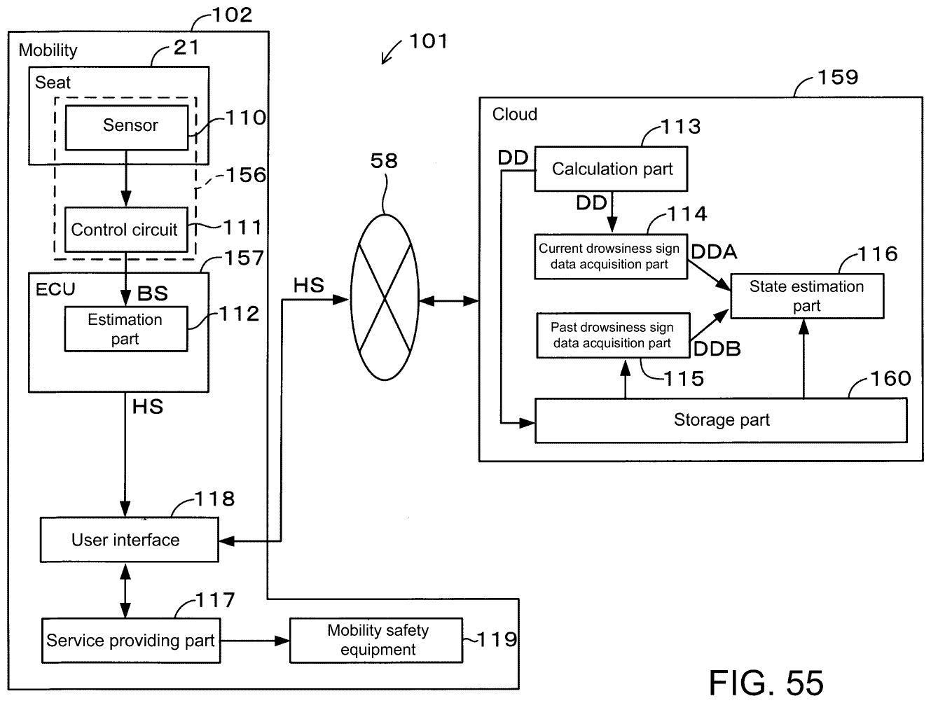

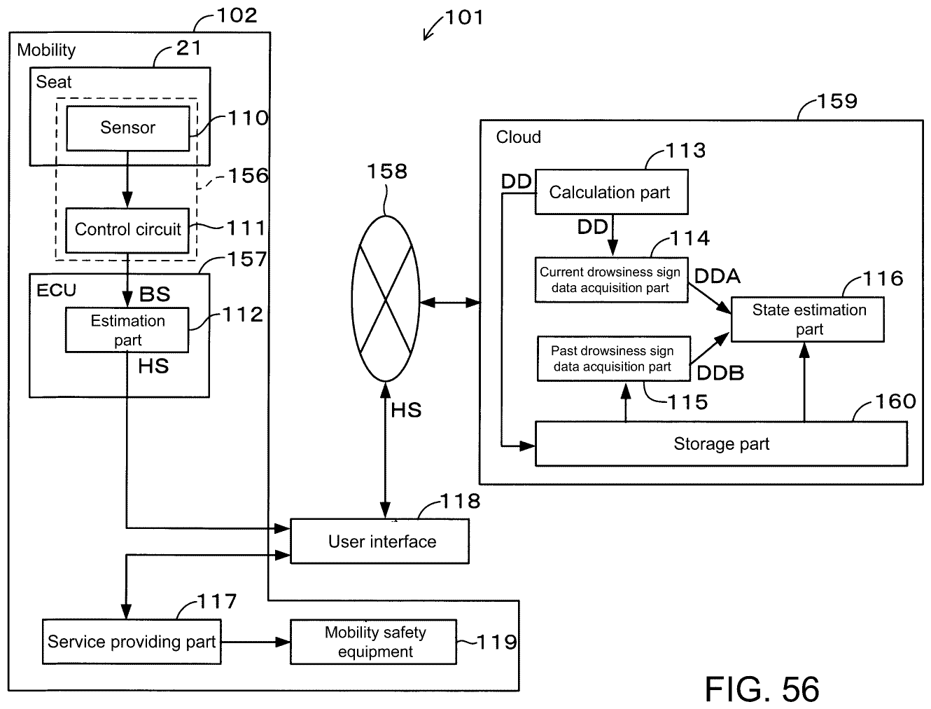

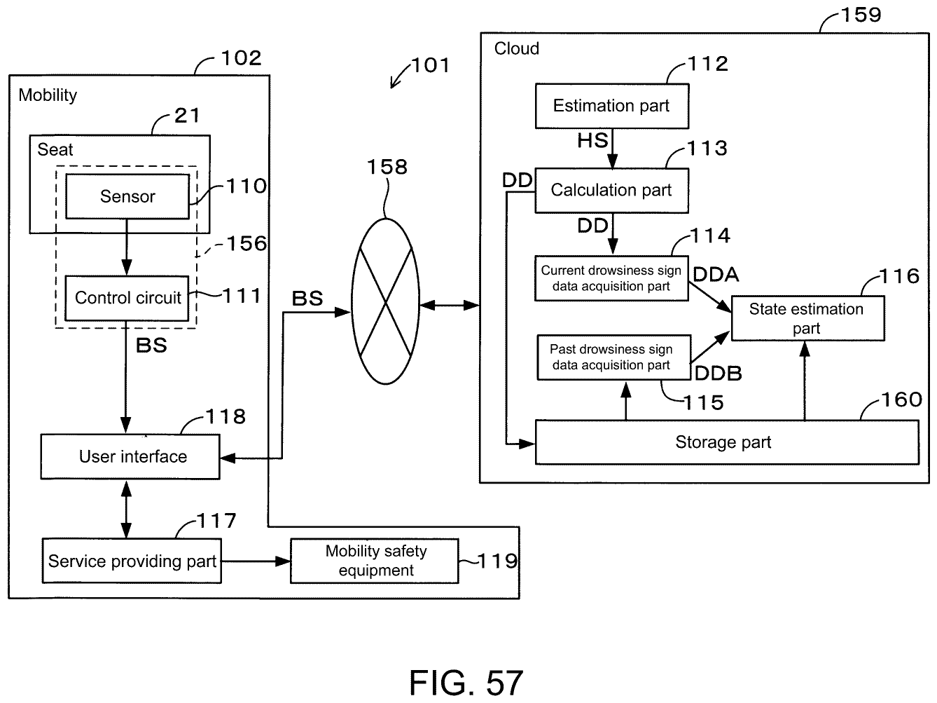

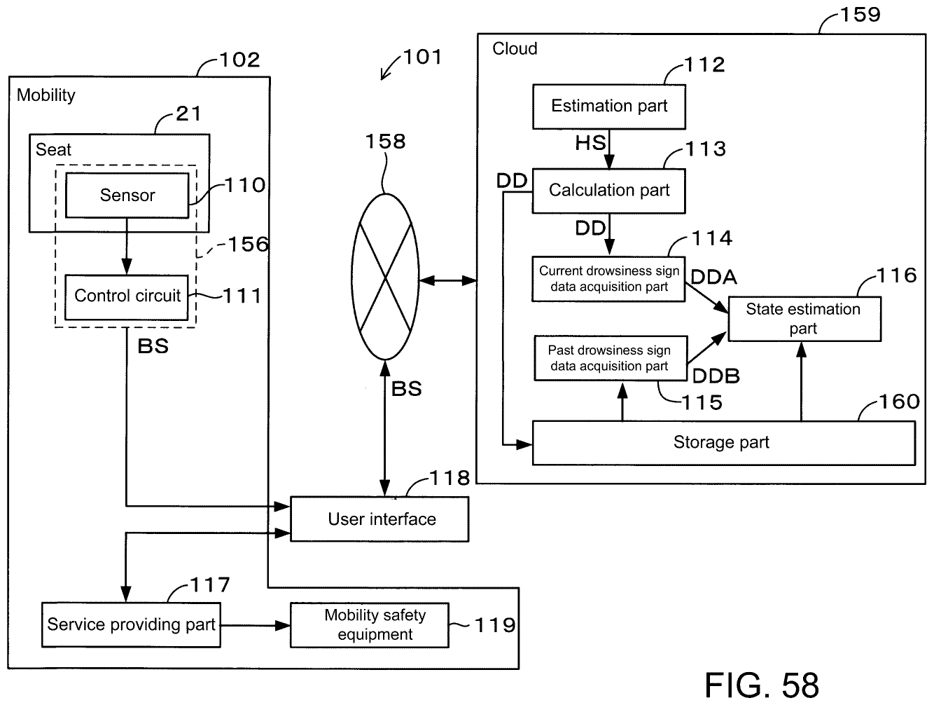

The disclosure is not limited to the above-described embodiments and may be applied to the following forms, for example, within a range that does not deviate from the spirit thereof.
(2.1) In Embodiment 2.1, the sensor 110 is configured to be attached to the seat 21 to acquire the biological signal BS from changes in body movement of the rider seated on the seat 21. However, the embodiment is not limited thereto, and the sensor 110 may also emit light (infrared light, ultraviolet light, laser light, visible light, etc.) or sound to the rider and detect reflection from the rider. In addition, the sensor 110 may also be a Doppler sensor that emits microwaves to the rider and detects changes in body movement of the rider from a difference between an emitted frequency and a received frequency, and any sensor 110 may be appropriately selected.
(2.2) In Embodiment 2.1, the sensor 110 is configured to be attached inside the seat surface part 22a. However, the embodiment is not limited thereto, and as shown in FIG. 50, the sensor 110 may also be configured as a component separate from the seat 21 placed on the upper surface of the seat surface part 22a, and attached to the seat surface part 22a by a conventional attachment means such as a rubber band or a belt. In addition, the sensor 110 may also be configured to be attached to the seat back part 22b by a conventional attachment means such as a rubber band or a belt.
(2.3) In Embodiment 2.1, the user interface 118 is a car navigation system assembled in the mobility 102. However, the embodiment is not limited thereto, and as shown in FIG. 51, the user interface 118 may also be a smartphone, a tablet terminal, a smartwatch, a dedicated terminal, etc. separate from the mobility 102. In FIG. 51 to FIG. 58, illustration of the data stored in the storage part 160 is omitted. According to this embodiment, the rider can receive specific services provided from the user interface 118 fixed to a holder (not shown) installed inside a vehicle compartment of the mobility 102. For example, a message about the health state of the rider may be conveyed by an image or a voice, or guidance or routes to sports facilities and the like may be displayed to the rider.
(2.4) In Embodiment 2.1, the user interface 118 transmits or receives information to or from the cloud 159 via the network 158. However, the embodiment is not limited thereto, and as shown in FIG. 52, the configuration disposed in the cloud 159 in Embodiment 2.1 may also be assembled as a control device 162 in the mobility 102.
(2.5) In Embodiment 2.1, the user interface 118 transmits or receives information to or from the cloud 159 via the network 158. However, the embodiment is not limited thereto, and as shown in FIG. 53, the ECU 157 may be configured to transmit drowsiness sign data DD to the cloud 159 via the network 158, and the user interface 118 (e.g., a car navigation system) assembled in the mobility 102 may be configured to provide information to the rider.
(2.6) In Embodiment 2.1, the user interface 118 transmits or receives information to or from the cloud 159 via the network 158. However, the embodiment is not limited thereto, and as shown in FIG. 54, the ECU 157 may be configured to transmit drowsiness sign data DD to the cloud 159 via the network 158, and the user interface 118 (e.g., a smartphone, a tablet terminal, or a smartwatch) separate from the mobility 102 may be configured to provide information to the rider.
(2.7) In Embodiment 2.1, the ECU 157 is configured to include the calculation part 113. However, the embodiment is not limited thereto, and as shown in FIG. 55, the cloud 159 may be configured to include the calculation part 113. In that case, the user interface 118 (e.g., a car navigation system) assembled in the mobility 102 may be configured to transmit or receive information to or from the cloud 159 via the network 158.
(2.8) In Embodiment 2.1, the ECU 157 is configured to include the calculation part 113. However, the embodiment is not limited thereto, and as shown in FIG. 56, the cloud 159 may be configured to include the calculation part 113. In that case, the user interface 118 (e.g., a smartphone, a tablet terminal, a smartwatch, or a dedicated terminal) separate from the mobility 102 may be configured to transmit or receive information to or from the cloud 159 via the network 158.
(2.9) In Embodiment 2.1, the mobility 102 is configured to include the estimation part 112 and the calculation part 113. However, the embodiment is not limited thereto, and as shown in FIG. 57, the cloud 159 may be configured to include the estimation part 112 and the calculation part 113. In that case, the user interface 118 (e.g., a car navigation system) assembled in the mobility 102 may be configured to transmit or receive information to or from the cloud 159 via the network 158.
(2.10) In Embodiment 2.1, the mobility 102 is configured to include the estimation part 112 and the calculation part 113. However, the embodiment is not limited thereto, and as shown in FIG. 58, the cloud 159 may be configured to include the estimation part 112 and the calculation part 113, and the user interface 118 (e.g., a smartphone, a tablet terminal, a smartwatch, or a dedicated terminal) separate from the mobility 102 may be configured to transmit or receive information to or from the cloud 159 via the network 158.

1. A health information service providing device (10) for a mobility user, comprising an acquisition device (56), an estimation part (13), a calculation part (14), a current VLF data acquisition part (15), a past VLF data acquisition part (16), a state estimation part (17), and a service providing part (18), wherein
the acquisition device (56) acquires a biological signal (BS) of the mobility user riding in a mobility (11);
the estimation part (13) estimates a heart rate signal (HS) related to a heart rate of the mobility user based on the biological signal;
the calculation part (14) calculates VLF data (VD) related to a very-low frequency component of 0.0033 to 0.04 Hz based on the heart rate signal;
the current VLF data acquisition part (15) acquires current VLF data (VDA), which is the VLF data of the mobility user this time including a current riding in a case of currently riding in the mobility or a most recent riding in a case of currently not riding in the mobility;
the past VLF data acquisition part (16) acquires past VLF data (VDB), which is a statistical representative value that is a numerical representative value or a positional representative value of the VLF data in a specific period in the past prior to this time;
the state estimation part (17) estimates a change in a health state of the mobility user based on a difference between the current VLF data and the past VLF data; and
the service providing part (18) provides a specific service to the mobility user based on the change in the health state of the mobility user.

2. The health information service providing device for a mobility user according to claim 1, wherein, in the case of currently riding in the mobility, the current VLF data is the statistical representative value of the VLF data in a period including at least a part of the current riding.

3. The health information service providing device for a mobility user according to claim 1, wherein, in the case of currently not riding in the mobility, the current VLF data is the statistical representative value of the VLF data in a period including at least a part of the most recent riding.

4. The health information service providing device for a mobility user according to claim 1, wherein the past VLF data comprises:
first past VLF data (VDF) which is the statistical representative value of the VLF data in a specific period one or more times prior to this time; and
second past VLF data (VDG) which is the statistical representative value of the VLF data in a specific period one or more times prior to the period of the first past VLF data, and
the state estimation part estimates the change in the health state of the mobility user based on a result of comparing the current VLF data and the first past VLF data according to a predetermined first comparison criterion (TVA, TVJ), and a result of comparing the current VLF data and the second past VLF data according to a predetermined second comparison criterion (TVA, TVK).

5. The health information service providing device for a mobility user according to claim 4, wherein the state estimation part is configured to:
estimate that the health state of the mobility user has changed in an unfavorable direction in a case where a first past difference value (ΔDVA) obtained by subtracting the current VLF data from the first past VLF data is equal to or greater than a threshold (TVA) which is an example of the first comparison criterion, and
further estimate that the health state of the mobility user has changed in an unfavorable direction in a case where the first past difference value is smaller than the threshold, and a second past difference value (ΔDVB) obtained by subtracting the current VLF data from the second past VLF data is equal to or greater than the threshold which is an example of the second comparison criterion.

6. The health information service providing device for a mobility user according to claim 4, wherein the state estimation part is configured to:
estimate that the health state of the mobility user has changed in an unfavorable direction in a case where a first past difference value obtained by subtracting the current VLF data from the first past VLF data is equal to or greater than a first past threshold (TVJ) which is an example of the first comparison criterion, and
further estimate that the health state of the mobility user has changed in an unfavorable direction in a case where the first past difference value is smaller than the first past threshold, and a second past difference value obtained by subtracting the current VLF data from the second past VLF data is equal to or greater than a second past threshold (TVK) which is a value greater than the first past threshold and is an example of the second comparison criterion.

7. The health information service providing device for a mobility user according to claim 5, wherein the current VLF data is the VLF data during the current riding or during the most recent riding,
the first past VLF data is the statistical representative value of the VLF data during a riding one or more times prior to the current riding or the most recent riding, and
the second past VLF data is the statistical representative value of the VLF data during a riding one or more times prior to the riding of the first past VLF data.

8. The health information service providing device for a mobility user according to claim 5, wherein the current VLF data is the VLF data during the current riding or during the most recent riding,
the first past VLF data is the statistical representative value of the VLF data during a riding one or more days ago, including a same time point as a time point at which the current VLF data is measured, and
the second past VLF data is the statistical representative value of the VLF data during a riding one or more days prior to the riding of the first past VLF data, including a same time point as the time point at which the current VLF data is measured.

9. The health information service providing device for a mobility user according to claim 5, wherein the current VLF data is the VLF data during the current riding or during the most recent riding,
the first past VLF data is the statistical representative value of the VLF data in a predetermined period prior to the current riding or the most recent riding, and
the second past VLF data is the statistical representative value of the VLF data in the predetermined period prior to the first past VLF data.

10. The health information service providing device for a mobility user according to claim 5, wherein the threshold comprises a normal threshold (TVE) and an alert threshold (TVF) which is a value smaller than the normal threshold, and
the state estimation part is configured to:
use the normal threshold in a case where the current VLF data exceeds a specific threshold (TVD), and
use the alert threshold in a case where the current VLF data is equal to or less than the specific threshold.

11. The health information service providing device for a mobility user according to claim 6, wherein the first past threshold comprises a first past normal threshold (TVL) and a first past alert threshold (TVM) which is a value smaller than the first past normal threshold,
the second past threshold comprises a second past normal threshold (TVN) and a second past alert threshold (TVO) which is a value smaller than the second past normal threshold, and
the state estimation part is configured to:
use the first past normal threshold and the second past normal threshold in a case where the current VLF data exceeds a specific threshold, and
use the first past alert threshold and the second past alert threshold in a case where the current VLF data is equal to or less than the specific threshold.

12. The health information service providing device for a mobility user according to claim 1, wherein the current VLF data is the VLF data of one-time riding during the current riding or the most recent riding,
the past VLF data is the statistical representative value of the VLF data of multiple-time ridings during a predetermined period in the past, and
the state estimation part estimates that the health state of the mobility user has changed in an unfavorable direction in a case where a difference value obtained by subtracting the current VLF data from the past VLF data is equal to or greater than a threshold.

13. The health information service providing device for a mobility user according to claim 1, wherein the current VLF data is the VLF data during the current riding or during the most recent riding,
the past VLF data is the statistical representative value of the VLF data during past riding including a same time point as a time point at which the current VLF data is measured, and
the state estimation part estimates that the health state of the mobility user has changed in an unfavorable direction in a case where a same-timeband difference value obtained by subtracting the current VLF data from the past VLF data is equal to or greater than a same-timeband threshold.

14. The health information service providing device for a mobility user according to claim 1, wherein the current VLF data is the statistical representative value of the VLF data in a predetermined comparison period including the current riding or the most recent riding,
the past VLF data is the statistical representative value of the VLF data in the comparison period prior to the current riding or the most recent riding, and
the state estimation part estimates that the health state of the mobility user has changed in an unfavorable direction in a case where a period difference value obtained by subtracting the current VLF data from the past VLF data is equal to or greater than a period threshold.

15. The health information service providing device for a mobility user according to claim 1, wherein the state estimation part estimates the change in the health state of the mobility user based on a tendency of the VLF data from the past to this time obtained from a plurality of the past VLF data and the current VLF data.

16. The health information service providing device for a mobility user according to claim 15, wherein the tendency comprises both or one of:
a state in which the plurality of the past VLF data monotonically decrease up to the current VLF data; and
a state in which the plurality of the past VLF data repeat cycles of decrease and increase up to the current VLF data.

17. The health information service providing device for a mobility user according to claim 1, wherein the current VLF data is the VLF data during the current riding or during the most recent riding,
the past VLF data comprises:
first past period VLF data (VDC) which is the statistical representative value of the VLF data in a first period prior to the current riding or the most recent riding; and
second past period VLF data (VDD) which is the statistical representative value of the VLF data in a second period longer than the first period of the first past period VLF data, and
the state estimation part is configured to:
estimate the health state of the mobility user by comprehensively considering a comparison result between the first past period VLF data and the current VLF data, and a comparison result between the second past period VLF data and the current VLF data.

18. The health information service providing device for a mobility user according to claim 17, wherein the state estimation part estimates that the health state of the mobility user has changed in an unfavorable direction in a case where
a first difference value (ΔDVF) obtained by subtracting the current VLF data from the first past period VLF data is equal to or greater than a first threshold (TVG), and
a second difference value obtained by subtracting the current VLF data from the second past period VLF data is equal to or greater than a second threshold.

19. The health information service providing device for a mobility user according to claim 17, wherein the state estimation part is configured to:
estimate that the health state of the mobility user has changed in an unfavorable direction in a case where a first difference value obtained by subtracting the current VLF data from the first past period VLF data is equal to or greater than a first threshold, and
further estimate that the health state of the mobility user has changed in an unfavorable direction in a case where the first difference value is smaller than the first threshold, and a second difference value (ΔDVG) obtained by subtracting the current VLF data from the second past period VLF data is equal to or greater than a second threshold (TVH).

20. The health information service providing device for a mobility user according to claim 1, further comprising a lowest VLF data acquisition part (61) acquiring lowest VLF data (VDE) which is a lowest value of the VLF data in a period longer than the specific period in the past of the past VLF data, wherein
the state estimation part is configured to:
estimate the change in the health state of the mobility user based on a difference between the current VLF data and the past VLF data, and
estimate that the health state of the mobility user has changed in an unfavorable direction in a case where the current VLF data is smaller than the lowest VLF data.

21. The health information service providing device for a mobility user according to claim 1, wherein the state estimation part is configured to:
estimate the change in the health state of the mobility user based on a difference between the current VLF data and the past VLF data, and
estimate that the health state of the mobility user has changed in an unfavorable direction in a case where the current VLF data is equal to or less than an emergency threshold (TVI).

22. A health information service providing device for a rider comprising an acquisition device (156), an estimation part (112), a calculation part (113), a current drowsiness sign data acquisition part (114), a past drowsiness sign data acquisition part (115), a state estimation part (116), and a service providing part (117), wherein
the acquisition device (156) acquires a biological signal (BS), which is a biological signal during riding, of a rider riding in a mobility (102);
the estimation part (112) estimates a heart rate signal (HS) related to a heart rate of the rider based on the biological signal;
the calculation part (113) calculates drowsiness sign data (DD) based on the heart rate signal, the drowsiness sign data (DD) being data related to a drowsiness sign of the rider and being a detection count of the drowsiness sign per unit time measured within a predetermined measurement time or a correlation value of the detection count;
the current drowsiness sign data acquisition part (114) acquires current drowsiness sign data (DDA), which is the drowsiness sign data of the rider in a state of currently riding in the mobility;
the past drowsiness sign data acquisition part (115) acquires past drowsiness sign data (DDB), which is the drowsiness sign data detected when the rider rode in the mobility in the past;
the state estimation part (116) estimates whether drowsiness of the rider has increased based on whether the current drowsiness sign data and the past drowsiness sign data satisfy a predetermined comparison condition (CC); and
the service providing part (117) executes control on providing equipment (118, 119) for providing a specific service to the rider, based on an estimation that the drowsiness of the rider has increased.

23. The health information service providing device for a rider according to claim 22, wherein the state estimation part is configured to:
estimate that the drowsiness of the rider has increased in a case where the current drowsiness sign data is equal to or greater than a drowsiness determination criterion (DCA), and
further estimate that the drowsiness of the rider has increased in a case where the current drowsiness sign data is less than the drowsiness determination criterion, and the current drowsiness sign data and the past drowsiness sign data satisfy the comparison condition.

24. The health information service providing device for a rider according to claim 22, wherein the state estimation part is configured to:
estimate that the drowsiness of the rider has increased in a case where the current drowsiness sign data is equal to or greater than a drowsiness determination criterion, and
further estimate that the drowsiness of the rider has increased in a case where the current drowsiness sign data is less than the drowsiness determination criterion, the current drowsiness sign data and the past drowsiness sign data satisfy the comparison condition, and the current drowsiness sign data is equal to or greater than a second drowsiness determination criterion (DCB) smaller than the drowsiness determination criterion.

25. The health information service providing device for a rider according to claim 23, wherein the current drowsiness sign data is the drowsiness sign data calculated based on the heart rate signal of the rider currently riding in the mobility,
the past drowsiness sign data comprises:
previous drowsiness sign data (DDC) which is the drowsiness sign data calculated based on the heart rate signal of the rider riding in the mobility last time; and
before-previous drowsiness sign data (DDD) which is the drowsiness sign data calculated based on the heart rate signal of the rider riding in the mobility a time immediately before last time, and
the comparison condition is that the current drowsiness sign data is greater than the previous drowsiness sign data, and the previous drowsiness sign data is greater than the before-previous drowsiness sign data.

26. The health information service providing device for a rider according to claim 23, wherein the current drowsiness sign data is the drowsiness sign data calculated based on the heart rate signal of currently riding in the mobility,
the past drowsiness sign data comprises:
previous drowsiness sign data which is the drowsiness sign data calculated based on the heart rate signal of the rider riding in the mobility last time; and
long-term drowsiness sign data (DDE) which is the drowsiness sign data per unit time calculated based on the heart rate signal of the rider riding in the mobility multiple times in the past, and
the comparison condition is that the current drowsiness sign data is greater than the previous drowsiness sign data, and the previous drowsiness sign data is greater than the long-term drowsiness sign data.

27. The health information service providing device for a rider according to claim 22, wherein the past drowsiness sign data is the drowsiness sign data calculated based on the heart rate signal of the rider riding in the mobility in a timeband in the past including a time point at which the current drowsiness sign data is measured.

28. The health information service providing device for a rider according to claim 22, wherein the current drowsiness sign data is the drowsiness sign data calculated based on the heart rate signal in a predetermined comparison period including the present time, and
the past drowsiness sign data is the drowsiness sign data calculated based on the heart rate signal in the comparison period prior to the present time.

29. The health information service providing device for a rider according to claim 22, wherein the service providing part executes control on the providing equipment to advance a timing of providing the service to the rider in a case where it is estimated that the drowsiness of the rider has increased.

30. The health information service providing device for a rider according to claim 22, wherein the past drowsiness sign data is a detection count of the drowsiness sign per unit time or a correlation value of the detection count, calculated based on the heart rate signal in a predetermined period longer than a period of the heart rate signal used for calculating the current drowsiness sign data.

31. The health information service providing device for a rider according to claim 22, wherein the service providing part executes control on a device that calls attention of at least one of a visual sense, an auditory sense, a tactile sense, and an olfactory sense of the rider as the providing equipment.

32. The health information service providing device for a rider according to claim 22, wherein the service providing part executes control on mobility safety equipment (119) related to safety of the mobility as the providing equipment.
